(19) United States
(12) Patent Application Publication
(10) Pub. No.: US 2025/0314643 A1
(43) Pub. Date: Oct. 9, 2025

(54) FLUORESCENT COMPLEXES COMPRISING TWO RHODAMINE DERIVATIVES AND A NUCLEIC ACID MOLECULE

(71) Applicants: CENTRE NATIONAL DE LA RECHERCHE SCIENTIFIQUE, Paris (FR); UNIVERSITE DE STRASBOURG, STRASBOURG (FR)

(72) Inventors: Michael RYCKELYNCK, STRASBOURG (FR); Farah BOUHEDDA, STRASBOURG (FR); Mayeul COLLOT, ILLKIRCH (FR); Andrii KLYMCHENKO, ILLKIRCH (FR); Tkhe Kyong FAM, STRASBOURG (FR)

(73) Assignees: CENTRE NATIONAL DE LA RECHERCHE SCIENTIFIQUE, Paris (FR); UNIVERSITE DE STRASBOURG, STRASBOURG (FR)

(21) Appl. No.: 17/617,037

(22) PCT Filed: Jun. 19, 2020
(86) PCT No.: PCT/EP2020/067239

§ 371 Date: Dec. 7, 2021

(30) Foreign Application Priority Data
Jun. 21, 2019 (EP) 19305806.2

(51) Int. Cl. G01N 33/53; C09K 11/06

(52) U.S. Cl. CPC G01N 33/5308; C09K 11/06; C09K 2211/1007; C09K 2211/1014; C09K 2211/1018; C09K 2211/1022; G01N 2458/00

(57) ABSTRACT

The invention relates to a molecular complex emitting fluorescent light comprising, or consisting essentially of: ⋅—a fluorophore, and ⋅—a nucleic acid molecule, wherein said fluorophore has of the following formula 3

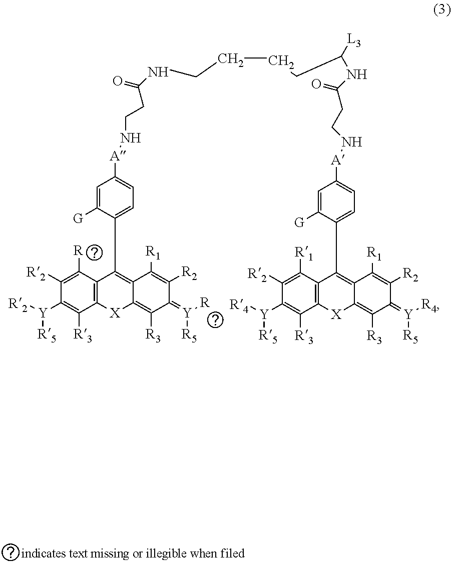

The present invention relates to fluorescent compounds, in particular for the detection of nucleic acids.

Cells constantly adapt their content to their needs, to changing environmental conditions or to pre-determined cell-cycles and differentiation programs by tuning their gene expression landscape. In addition, live-cell imaging of protein-coding gene expression demonstrated that significant cell-to-cell variation in gene expression occurs even within population of isogenic cells within the same environment. Currently, imaging of gene expression in live cells relies mainly on proteins genetically modified with either fluorescent proteins or tags for specific chemical labelling. RNA is also an important actor that orchestrates key steps of gene expression regulation. However, converser to protein, no naturally fluorescent RNA has been discovered yet, making urgent the need for technologies enabling live-cell RNA monitoring with single-cell resolution and leading to the development of a palette of RNA detection methodologies especially imaging technologies.

The first breakthrough in live-cell RNA imaging came with the use of RNA-binding proteins (RBP) fused to fluorescence proteins (FP). In these completely genetically encoded systems, an array (tens of repeats) of the RNA RBP-binding motif is incorporated into the 3′ untranslated region of the target messenger RNA (mRNA). Co-expressing the gene coding for the corresponding RBP-FP in the same cell allowed tracking target RNA upon its decoration with FP. This methodology has enabled collecting important data on gene expression and RNA trafficking and remained so far, the reference method. Substantial simplification of the approach is possible by using RNA-based fluorogenic modules in which bulky FPs are substituted by small fluorogens, i.e. dyes lighting up their fluorescence upon interaction with a target (bio)molecule”. In this case, target mRNA is modified by the insertion of a specific nucleic acid sequence, so-called “light-up RNA aptamer”, able to fold to form a binding pocket where fluorogen turns on its fluorescence.

Capacity of RNA to light-up fluorogenic dyes was first established with an aptamer interacting specifically with Malachite Green, but the toxicity of the radicals produced upon complex illumination limited its use for live-cell applications. Later on, Jaffrey's lab introduced the cell permeable and non-toxic GFP-mimicking fluorogen 3,5-difluoro-4-hydroxybenzylidene imidazolinone (DFHBI), together with Spinach, an RNA aptamer able to bind and strongly activate DFHBI fluorescence. Further derivatives of the aptamer (i.e. Spinach2 and Broccoli) and of the corresponding fluorogen (i.e. DFHBI-1T) were later developed by the same lab and started to revolutionize RNA live-cell imaging by making possible to set-up a whole range of imaging-based applications. Unfortunately, DFHBI-based modules present limited brightness and photostability because of their rapid photoisomerization, making them less suited for low-abundant RNA detection and extended imaging time. Substantial gain in photostability and brightness was achieved by using fluorogens based on classical organic dyes (e.g. cyanines and rhodamines), including those operating by Photoinduced Electron Transfer (PET) or Forster Resonance Energy Transfer (FRET) mechanisms. For instance, conjugates of sulforhodamine B dye (SRB) with dinitroaniline (DN) PET quencher turn on their fluorescence upon association with an aptamer binding the SRB (e.g. SRB-2 aptamer) or the DN moiety. An alternative strategy, which could significantly improve brightness of the fluorogen, is to use a homo- or hetero-dimer of dyes that self-quenches in aqueous solution but becomes fluorescent upon dimer opening after binding to the target biomolecule. So far, this concept has yielded probes for detecting ligand-receptor interaction or DNA hybridization, but it has not been proposed for designing fluorogens activated by light-up RNA aptamers. Brightness and photostability also rely on the aptamer itself as nicely illustrated by Corn, an aptamer that recognizes and activates the fluorescence of the 3,5-difluoro-4-hydroxybenzylidene imidazolinone-2-oxime (DFHO). In this case, the fluorogen is caged in a pocket formed by two RNA monomers, which protects it from rapid photoinactivation, conferring the module an impressive photostability.

Light-up aptamers are usually isolated by a Systematic Evolution of Ligand by Exponential enrichment (SELEX) approach, a powerful technology for selecting aptamers with very high affinity and selectivity for their target, as exemplified by Mango RNA, a light-up aptamer binding its fluorogen (the biotinylated Thiazole Orange-1 or TO1-biotin) with nanomolar affinity. However, SELEX does not select molecules for their fluorogenic capacities, a limitation that can be overcome by the use of a functional screening.

For instance, using microfluidic-assisted in vitro compartmentalization (pIVC), allowed to recently identify mutants of the Spinach and Mango aptamers displaying both improved brightness and folding efficiency as illustrated in the international application WO2018198013.

However, live-cell imaging of RNA remains a challenge because RNA aptamers that can light-up small fluorogenic dyes could still suffer from poor brightness and photostability.

The present invention intends to obviate these drawbacks.

One aim of the invention is to provide new and efficient means allowing to live-cell imaging of RNA with enhanced brightness and photostability.

The invention relates to a molecular complex emitting fluorescent light comprising, or consisting essentially of a fluorophore, and a nucleic acid molecule,
wherein said fluorophore has of the following formula 1

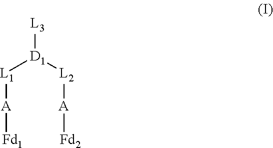

wherein
   independently from each other, Fd1 and Fd2 are fluorescent dyes,
   D1 represents a group chosen from:

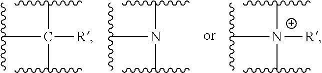

or from a cyclo(C₃-C₇)alkyl, a monocyclic aromatic group, heterocyclic group or a monocyclic non-aromatic, alkane or heterocyclic group, wherein R′ represents a hydrogen atom or a (C₁-C₈)alkyl, linear or cyclic, saturated or not,
   independently from each other, L1 and L2 is covalently bound to D1, is a group consisting of a single bond; a linear or branched alkyl group having from 1 to 24 carbon atoms (C₁-C₂₄), at least one of said carbon atoms being replaced by an heteroatom, e.g. O, N, S, or not, said alkyl group being substituted or not by an amido, an amino, a keto, an oxy or a carboxyl group or a linear or branched unsaturated or not alkyl group having from 2 to 24 carbon atoms, at least one of said carbon atoms being replaced by an heteroatom e.g. O, N, S, or not, said alkyl group being substituted or not by an amido, an amino, a keto, an oxy, a carboxyl group;
   L3 is a hydrogen atom or corresponds to L1 or L2, i.e. a linear or branched alkyl group having from 1 to 24 carbon atoms (C₁-C₂₄), at least one of said carbon atoms being replaced by an heteroatom, e.g. O, N, S, or not, said alkyl group being substituted or not by an amido, an amino, a keto, an oxy or a carboxyl group or a linear or branched unsaturated or not alkyl group having from 2 to 24 carbon atoms, at least one of said carbon atoms being replaced by an heteroatom e.g. O, N, S, or not, said alkyl group being substituted or not by an amido, an amino, a keto, an oxy, a carboxyl group, possibly substituted by a functionalizable moiety, e.g. azide, alkyne, DBCO, active ester, carboxylic acid, maleimide group or a functional molecule such as a ligand or a biomolecule e.g. biotin, or desthiobiotin, and
   A is a C₁-C₁₂ alkyl, linear or cyclic, possibly substituted by an aryl, preferably a phenyl, substituted or not,
said fluorophore being submitted to quenching or energy transfer when it is not associated to said nucleic acid molecule in aqueous solution, or said fluorophore being submitted to quenching or energy transfer when considered alone in aqueous solution,
wherein said nucleic acid molecule is able to activate the fluorescence of said fluorophore in an aqueous solution, when interacting with said fluorophore, and
wherein said nucleic acid molecule is able to specifically interact, in a sequence specific manner, with said fluorophore.

The inventors unexpectedly identified molecular complex comprising essentially a fluorophore and a nucleic acid molecule that is soluble in aqueous solution, can be used in cell culture and in vivo, harbours high brightness properties and is only activatable when both compounds interact together.

The compounds that constitute the complex, are therefore the fluorophore and the nucleic acid molecule.

### Fluorophore

The fluorophore of the complex described above is a fluorophore of formula 1,

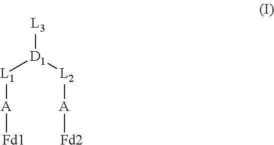

and contains two fluorescent dyes Fd1 and Fd2 that can be identical or different.

Both Fd1 and Fd2 are dyes that can re-emit light upon light excitation. Fd1 and Fd2 typically contain several combined aromatic groups, or planar or cyclic molecules with several π bonds. It can be coumarins, pyrenes, cyanines, BODIPYs, merocyanines an their derivatives well known in the art. It is advantageous the Fd1 and Fd2 be xanthene derivatives such as fluorescein dye, rhodamine dye, sulforhodamine dye, Oregon green dye, eosin dye, and Texas red dye, silicon-rhodamine dye, or one of their derivatives well known in the art.

Due to the structure of the fluorophore, both Fd1 and Fd2 dyes are chemically linked to each other and, depending upon the environmental conditions can be close together. This results in a decrease of the fluorescence intensity, or an absence of fluorescence at the emitting wavelength, when both dyes are excited at the specific wavelength. This phenomenon is the quenching or energy transfer.

Thus, when the fluorophore is in an environment, e.g. aqueous solution, that induces the rapprochement of both dyes, quenching occurs and no fluorescence, or a decreased fluorescence, is emitted by the fluorophore when excited at the appropriated wavelength. On the contrary, when the fluorophore is in an appropriate environment, e.g. organic solvent, Fd1 and Fd2 are far from each other and the quenching does not occur.

Based on these properties, the inventors engineered a strategy to specifically activate the fluorescence of said fluorophore, when the fluorophore is in aqueous solution, i.e. when the fluorophore is in physiological conditions to be used in living cells.

The inventors identified that nucleic acid molecules can specifically interact with said fluorophore, such that:
the fluorescence is enhanced compared to the fluorescence of the fluorophore, when it does not interact with said nucleic acid molecule, or when said fluorophore is placed alone in an organic solvent that does not induce quenching, and
the interaction is very specific with a high affinity.

In the fluorophore described above, L3 represents a functionalizable moiety that can be used to detect, isolate or purify the fluorophore.

L1 and L2 correspond to the “arms” of the fluorophore that associate to each other Fd1 and Fd2 dyes. L1 and L2 are covalently linked to each other via D1, as defined above.

L1 and L2 independently from each other can be:
either a single bound, such that the fluorophore will have the following formula

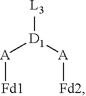

when both L1 and L2 are a single bound,
or a linear or branched alkyl group having 1, or, 2, or 3, or 4, or 5, or 6, or 7, or 8, or 9, or 10, or 11, or 12, or 13, or 14, or 15, or 16, or 17, or 18, or 19, or 20, or 21, or 22, or 23 or 24 carbon atoms
or a linear or branched alkyl group having 1, or, 2, or 3, or 4, or 5, or 6, or 7, or 8, or 9, or 10, or 11, or 12, or 13, or 14, or 15, or 16, or 17, or 18, or 19, or 20, or 21 n, or 22, or 23 or 24 carbon atoms, wherein at least one carbon atom is substituted by an hetero atom, e.g. O, N or S,
or a linear or branched alkyl group having 1, or, 2, or 3, or 4, or 5, or 6, or 7, or 8, or 9, or 10, or 11, or 12, or 13, or 14, or 15, or 16, or 17, or 18, or 19, or 20, or 21 n, or 22, or 23 or 24 carbon atoms, said alkyl group being itself substituted by an amido, an amino, a keto, an oxy, a carboxyl group, a linear or branched unsaturated or not alkyl group having from 2 to 24 carbon atoms,
or a linear or branched alkyl group having 1, or, 2, or 3, or 4, or 5, or 6, or 7, or 8, or 9, or 10, or 11, or 12, or 13, or 14, or 15, or 16, or 17, or 18, or 19, or 20, or 21 n, or 22, or 23 or 24 carbon atoms, wherein at least one carbon atom is substituted by an hetero atom, e.g. O, N or S, the carbon and/or the heteroatoms of said alkyl group being themselves substituted by an amido, an amino, a keto, an oxy, a carboxyl group, a linear or branched unsaturated or not alkyl group having from 2 to 24 carbon atoms.
In the fluorophore, A represents a C₁-C₁₂ alkyl, i.e. a C₁, a C₂, a C₃, a C₄, a C₅, a C₆, a C₇, a C₈, a C₉, a C₁₀, a C₁₁, or a C₁₂ alkyl, or a C₁-C₁₂ alkyl substituted by an aryl group, said aryl being substituted or not.

### Nucleic Acid Molecule.

In the complex disclosed above, the nucleic acid molecule interacts with the fluorophore such that it inhibits or avoids quenching that occurs between both Fd1 and Fd2 dyes. This interaction is specific of the nucleic acid molecule sequence, such that the nucleic acid molecule should advantageously have a determined nucleic acid sequence to interact with said fluorophore.

The nucleic acid molecule in the invention is a Deoxyribonucleotide molecule (DNA molecule), a Ribonucleotide molecule (RNA molecule), or any derived nucleic acid molecules such as XNA, Spiegelmer molecule (or L-RNA molecules), or molecules comprising 2′Fluoro, or 2′ Methoxy nucleotides. The nucleic acid molecule is preferably a ribonucleic acid molecule (RNA molecule) that can adopt a specific three-dimensional conformation allowing the activation of the fluorophore submitted to quenching or energy transfer. This nucleic acid molecule is in particular an aptamer, having a high affinity to said fluorophore, and which induce a high brightness of the fluorophore further to the interaction.

In the invention, the nucleic acid molecule can contain advantageously a sequence that is repeated once, i.e. the nucleic acid contain a repeat of a determined sequence.

Advantageously, the invention relates to the molecular complex as defined above, wherein Fd1 and Fd2 are represented by formula 2:

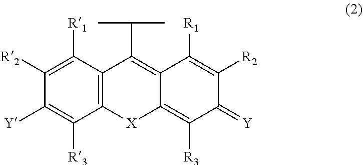

Wherein
X is NH, C(R)₂, O, Si(R)₂Ge(R)₂Sn(R)₂P(R)₂B(R)₂S, SO₂, Se, Te, TeO, wherein R can be alkyl or aromatic groups, or O, O-alkyl, sulfonyl such as sulfonate (SO3-) or sulfonamide;
Y is O, N—R₆ or

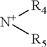

Y′ is O—R′₆ or N—R′₇ or

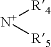

R₁ and R′₁ independently from each other, are H, a halogen atoms or a (C₁-C₁₈) alkyls, linear or cyclic, possibly branched,
R₂, R′₂, R₃, R′₃ can be H, sulfonyl such as sulfonate (SO3-) or sulfonamide;
R₂ and R₄ may form, together with the atoms of the carbon cycle to which R₂ is connected to, at least one fused aromatic heterocycle, said heterocycle cycle having 5 to 9 atoms,
R′₂ and R′₄ may form, together with the atoms of the carbon cycle to which R′₂ is connected to, at least one fused aromatic heterocycle, said heterocycle cycle having 5 to 9 atoms,
R₅ and R₃ may also form, together with the atoms of the carbon cycle to which R₃ is connected to, at least one fused aromatic heterocycle, said heterocycle cycle having 5 to 9 atoms,
R′₅ and R′₃ may also form, together with the atoms of the carbon cycle to which R′₃ is connected to, at least one fused aromatic heterocycle, said heterocycle cycle having 5 to 9 atoms,
R₄ and R₅ may also form at least one fused aromatic heterocycle, said heterocycle cycle having 3 to 9 atoms,
R′₄ and R′₅ may also form at least one fused aromatic heterocycle, said heterocycle cycle having 3 to 9 atoms, and
R₄, R′₄, R₅, R′₅, R₆ and R′₇, independently from each other, are polymethylene unit having 1 carbon to about 20 carbons, inclusive, optionally comprising at least one hetero atom selected from N, O and S.

More advantageously, the invention relates to the above mentioned molecular complex, wherein said fluorophore has the following formula 3:

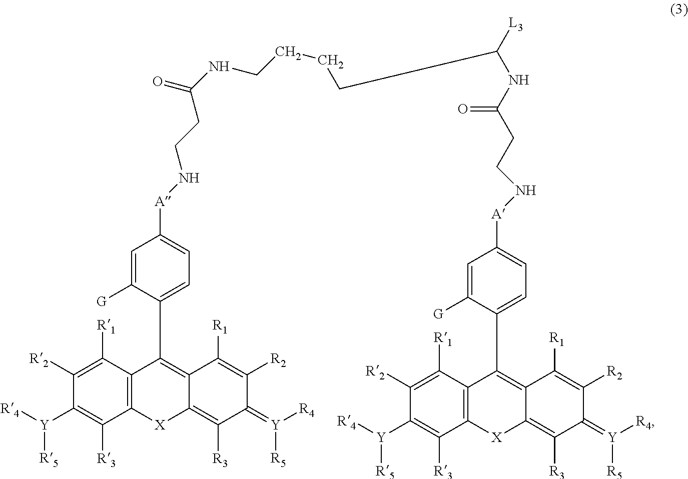

in particular formula 3-1

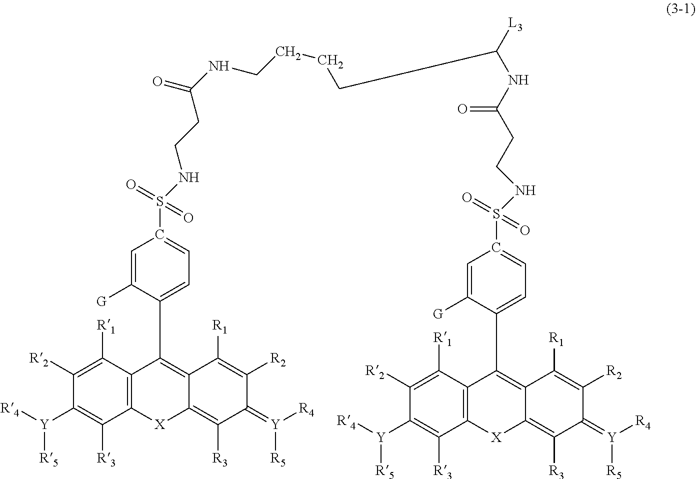

Wherein R₁, R′₁, R₂, R′₂, R₃, R′₃, R₄, R′₄, R₅, R′₅, R₆ and R′₇ and L₃ are as defined above, and A′ and A″ are independently from each other ether bond, ester, thioether, thioester, amide, sulfonamide, carbamate, thiocarbamate urea or thiourea,

Wherein G is H, an alkane (CH3), amido, an amino, a keto, an oxy, a carboxyl, a sulfo, sulfonyl or sulfonate group), a halide atom.
G can be in ortho, or meta or para position and can be repeated on the benzyl cycle.
A′ and A″ can be in ortho, meta or para position

More advantageously, the invention relates to the molecular complex as defined above, wherein said -A-Fd1 and -A-Fd2 groups are one of the following fluorophores: Rhodamine, Sulfo-Rhodamine, non-N-Alkylated Rhodamine, Ethyl-alkylated rhodamine, fluorescein, Silicon-Rhodamine, or carborhodamine.

In one advantageous embodiment, the invention relates to the molecular complex as defined above, wherein said fluorophore is one of the following compounds:

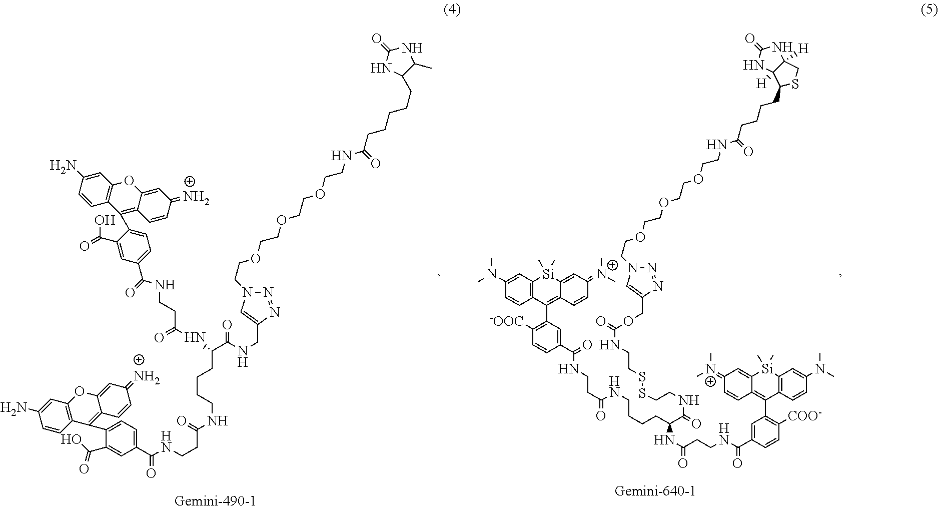

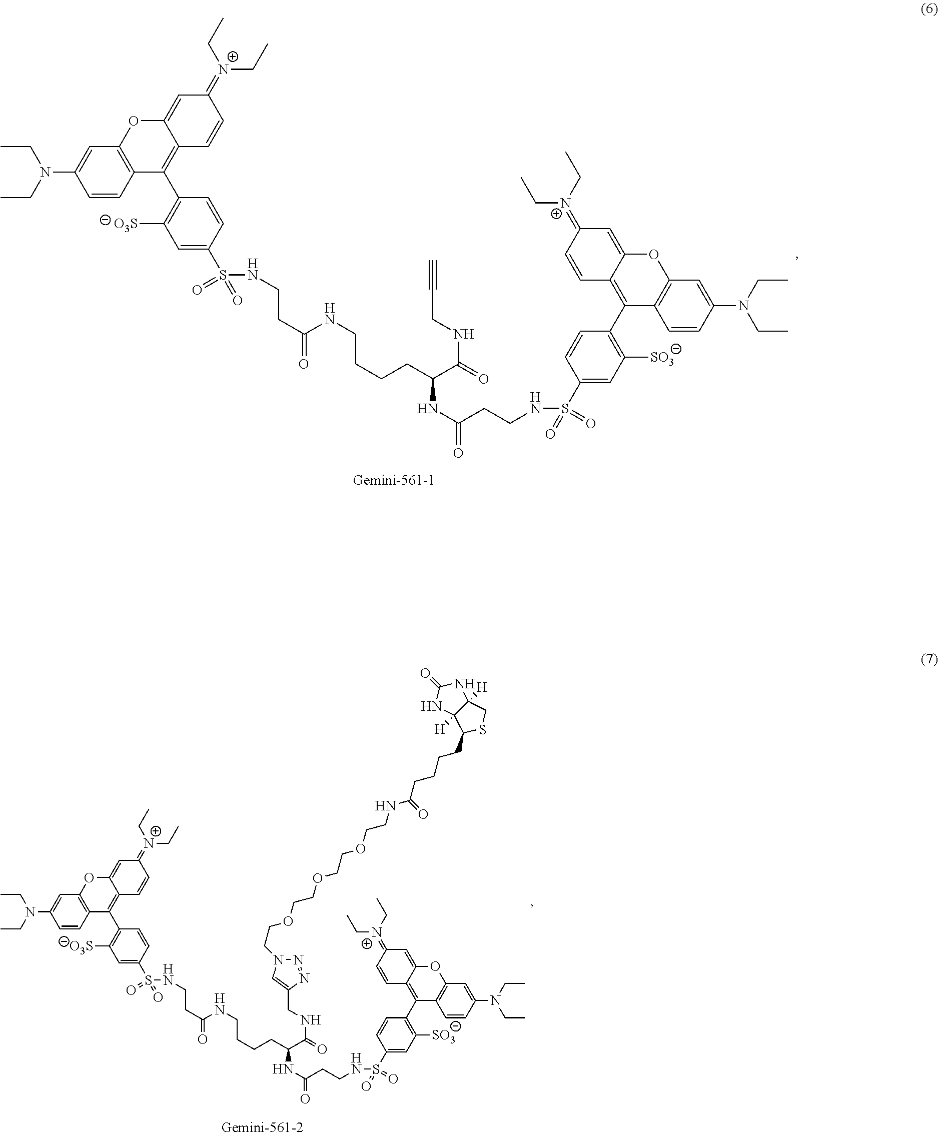

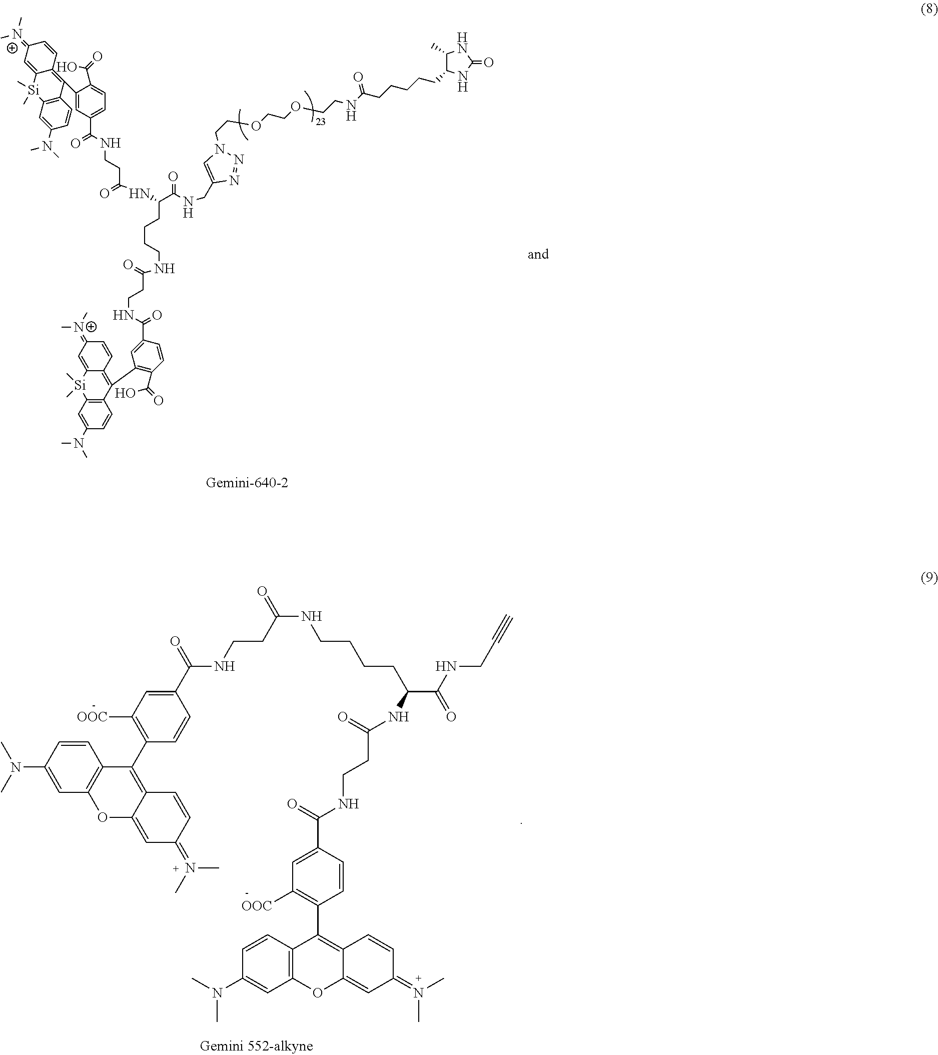

In another advantageous embodiment, the invention relates to the above mentioned molecular complex, wherein said complex harbors a fluorescence intensity at least 3-fold higher compared to the fluorescence intensity of corresponding free uncomplexed fluorophore in aqueous medium and wherein said nucleic acid molecule has an affinity quantified by a Kd value of at most 500 nM, preferably lower, for said fluorophore.

In the invention, affinity has its common sense well known in the art, the tendency of a chemical species to react with another species to form a chemical compound. Affinity can also be referred to as the tendency of certain atoms (or molecules) to aggregate or bond together, and includes electrostatic interactions, hydrogen bounds,

The term “specifically binding”, “specifically binds” or “specifically interacts” is used herein to indicate that this moiety has the capacity to recognize and interact specifically with the molecular target of interest, while having relatively little detectable reactivity with other structures present in the aqueous phase such as other molecular targets that can be recognized by other probes. There is commonly a low degree of affinity between any two molecules due to non-covalent forces such as electrostatic forces, hydrogen bonds, Van der Waals forces and hydrophobic forces, which is not restricted to a particular site on the molecules and is largely independent of the identity of the molecules. This low degree of affinity can result in non-specific binding. By contrast when two molecules bind specifically, the degree of affinity is much greater than such non-specific binding interactions. In specific binding a particular site on each molecule interacts, the particular sites being structurally complementary, with the result that the capacity to form non-covalent bonds is increased. The term “sequence-specific” binding or interaction refers to specific binding of a molecule to a nucleic acid of a given sequence, whereas the mentioned molecule cannot bind to nucleic acids of other sequences.

The fluorescence enhancement can be measured by a fluorometer and can be obtained by dividing the maximum fluorescence intensity of the fluorophore alone in aqueous medium by the maximum fluorescence intensity of the fluorophore in the presence of the said nucleic acid in the same medium an at the same concentration.

The Kd value can be obtained by measuring the fluorescence intensity of the fluorophore in aqueous medium with increasing amount of the said nucleic acid. The plot of the fluorescence intensity versus the concentration of the said nucleic acid will provide the Kd value after fitting with the proper equation (example: Hill equation).

The change in the brightness and Kd values can be acquired using standard fluorescence spectrometer, where the complex and the fluorophore alone are measured in aqueous medium in a cuvette.

The affinity of a molecule X for its partner Y can generally be represented by the dissociation constant (Kd). In preferred embodiments, the Kd representing the affinity between the capture moiety and the molecular target of interest is from 1·10⁻⁷M or lower, preferably from 1·10⁻⁸M or lower, and even more preferably from 1·10⁻⁹M or lower. Specificity and affinity can be relatively determined by binding or competitive assays, using e.g., Biacore instruments.

Advantageously, the invention relates to the above-mentioned molecular complex, wherein said nucleic acid molecule comprises a first and a second region, said first and second regions being such that:
the first region comprises the nucleotide sequence of SEQ ID NO: 1; (UGAUGGA) and
the second region comprises the nucleotide sequence of SEQ ID NO: 2 (CAAGGUUAAC),
   provided that said nucleic acid molecules is not the nucleic acid molecule consisting of the sequence as set forth in SEQ ID NO: 3 (SRB2) 5′-GGGAGACAGCUAGAGUACGGAACCUCGCUUCGGCGAUGAUGGACAGGUUCC GACACGAGCACAGUGUAC-3′, the above proviso does not concern the molecules of SEQ ID NO: 3 having at least one modified nucleotides, such as 2′-fluoro nucleotides.

Advantageously, the nucleic acid molecule as defined above comprises 2 sequences SEQ ID NO: 1 and two sequences SEQ ID NO: 2.

For instance the nucleic acid molecule comprises:
the first region comprises the nucleotide sequence of SEQ ID NO: 1; (UGAUGGA), repeated twice and
the second region comprises the nucleotide sequence of SEQ ID NO: 2 (CAAGGUUAAC), or
the first region comprises the nucleotide sequence of SEQ ID NO: 1; (UGAUGGA), and
the second region comprises the nucleotide sequence of SEQ ID NO: 2, repeated twice, or
the first region comprises the nucleotide sequence of SEQ ID NO: 1; (UGAUGGA), repeated twice and
the second region comprises the nucleotide sequence of SEQ ID NO: 2 (CAAGGUUAAC), repeated twice.

The first and the second regions of SEQ ID NO: 1 and SEQ ID NO: 2 constitute the minimal essential domain of the nucleic acid molecule responsible of the activation of the fluorescence properties of said fluorophore in aqueous solution.

Advantageously, the invention relates to the nucleic acid molecule as defined above, said nucleic acid molecule being a linear single-stranded molecule, a circular single-stranded molecule or a two-stranded molecule.

As disclosed in the art, the nucleic acid according to the invention may be a linear single-stranded molecule. The sequences SEQ ID NO: 1 and SEQ ID NO: 2 are separated from each other in the same molecule but are close to each other when the molecule acquires its final tridimensional conformation.

Moreover, the nucleic acid molecule can be a circular single-stranded molecule. In this case, the molecule has the same structure than a linear single-stranded molecule, except that the 5′- and 3′-ends are linked by a phosphodiester bond.

The nucleic acid molecule according to the invention can also be constituted by two separated molecules, the first one containing the sequence SEQ ID NO: 1 and the second one containing the sequence SEQ DI NO: 2, these two molecules being close to each other to confer the molecule a structure similar to the structure adopted by a single stranded molecule.

In the invention, when the nucleic acid molecule is a single stranded linear or circular molecule, both sequences are contained in the same molecule. By contrast, when the aptamer is constituted by two different single stranded molecules, each sequence is contained in one specific molecule, i.e. the two sequences are advantageously not contained by the same single stranded molecule.

Advantageously, the invention relates to the above molecular complex, wherein the nucleic acid molecule comprises one of the nucleotide sequences of

(SEQ ID NO: 4)
-(N)ₐUGAUGGA(N)_{b}CAAGGUUAAC(N)ₐ,
(SEQ ID NO: 5)
-(N)ₐCAAGGUUAAC(N)_{b}UGAUGGA(N)ₐ,

or
the two following sequences

(SEQ ID NO: 6)
-(N)ₐUGAUGGA(N)_{b},
(SEQ ID NO: 7)
(N)ₐCAAGGUUAAC(N)_{b},

wherein a, b and c are integer
a is higher than or equal to 4, preferably varies from 4 to 100,
b is higher than or equal to 1, preferably varies from 3 to 50,
c is higher than or equal to 1, preferably varies from 1 to 200,
or any variant of said nucleic acid molecule by substitution of at least one nucleic acid of one at least of said sequences SEQ ID NO: 4, SEQ ID NO: 5, SEQ ID NO: 6 and SEQ ID NO: 7, provided that said variant retains the ability to interact with said fluorophore and is able to induce fluorescence in aqueous solution.

In the above sequences, a varies from 1 to 100, which means that a can be equal to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100.

In the above sequences, b varies from 1 to 50, which means that b can be equal to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50.

In the above sequences, c varies from 4 to 200, which means that c can be equal to 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 and 200.

In one advantageous embodiment, the invention relates to the above-defined molecular complex, wherein the nucleic acid molecule comprises, or consists essentially of, or consists of one of the nucleotide sequences as set forth in SEQ ID NO: 8, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27 and SEQ ID NO: 29.

represents o-Coral
SEQ ID NO: 8
5′-GGGAGACAGCUAGAGUACAGGAACCCCGCUUCGGCGGUGAUGGAGAGGCGC
AAGGUUAACCGCCUCAGGUUCCGGUGACGGGGCCUCGCUUCGGCGAUGAUGG
AGAGGCGCAAGGUUAACCGCCUCAGGUUCUGACACGAGCACAGUGUAC-3′
represents 4C10
SEQ ID NO: 9
5′-AGAACCCCGCUUCGGCGGUGAUGGAGAGGCGCAAGGUUAACCGCCUCAGGU
UCC(N)_{d}GGGGCCUCGCUUCGGCGAUGAUGGAGAGGCGCAAGGUUAACCGCCUC
AGGUUCU-3′, wherein N represents A, U, G or C, and d vary from 18 to 60 nucleotides,
represents 4C38
SEQ ID NO: 10
5′-GGAACCUCGCUUCGGCGAUGAUGGAGAGGCGCAAGGUUAACCGCC-CCGGU
UCC(N)_{d}GGAACCCCGCUUCUGCGGUGAUGGAGAGGCGCAAUGUUAACCGCCUC
AGGUUCC-3′, wherein N represents A, U, G or C, and d vary from 18 to 60 nucleotides,
represents 4C31
SEQ ID NO: 11
5′-AGAACCCCGCUUCGGCGGUGAUGGAGAGGCGCAAGGUUAACCGCCUCAGGU
UCC(N)_{d}GGGGCCUCGCUUCGGCGAUGAUGGAGAGGCGCAAGGUUAACCGCCUC
AGGUUCC-3′, wherein N represents A, U, G or C, and d vary from 18 to 60 nucleotides,
represents 4C11
SEQ ID NO: 12
5′-GGAACCUCGCUUCGGCGAUGAUGGAGAGGCGCAAGGUUAACCGCCUCAGGU
UCC(N)_{d}GGAACCUCGCUUCGGCGAUGAUGGAGAGGCGCAAGGUUAACCGCCUC
AGGUUCC-3′, wherein N represents A, U, G or C, and d vary from 18 to 60 nucleotides,
represents 4C31
SEQ ID NO: 13
5′-GGAACUUCGCUUCGGCGAUGAUGGAGAGGCGCAAGGUUAACCGCCUCAGGU
UCC(N)_{d}GGAACUUCGCUUCGGCGAUGAUGGAGAGGCGCAAGGUUAACCGCCUC
AGGUUCC-3′, wherein N represents A, U, G or C, and d vary from 18 to 60 nucleotides,
represents 4C5
SEQ ID NO: 14
5′-GGGACCCCGCUUCGGCGGUGAUGGAGAGGCGCAAGGUUAACCGCCUCAGGU
UCC(N)_{d}GGAACCUCGCUUCGGCGAUGAUGGAGGGGCGCAAGGUUAACCGCCUC
AGGUUUC-3′, wherein N represents A, U, G or C, and d vary from 18 to 60 nucleotides,
represents 4C12
SEQ ID NO: 15
5′-GGAGCCCCGCUUCGGCGGUGAUGGAGAGGCGCAAGGCUAACCGCCUC-GGU
UCC(N)_{d}GGAGCCUCGCUUCGGCGAUGAUGGAGAGGCGCAAGGUUAACCGCCUC
AGGUUCC-3′, wherein N represents A, U, G or C, and d vary from 18 to 60 nucleotides,
represents 4C33
SEQ ID NO: 16
5′-GGAACCUCGCUUCGGCGAUGAUGGAGAGGCGCAAGGUUAACCGCCUCAGGU
UCC(N)_{d}GGAGCCUCGCUUCGGCGAUGAUGGAGGGGCGCAAGGUUAACCGCCUC
AGGUUCA-3′, wherein N represents A, U, G or C, and d vary from 18 to 60 nucleotides,
represents 4C17
SEQ ID NO: 17
5′-GGAACCUCACUUCGGUGAUGAUGGAGAGGCGCAAGGUUAACCGCCUCAGGU
UCC(N)_{d}GGAGCCUCGCUUCGGCGAUGAUGGAGAGGCGCAAGGUUAACCGCCUC
AGGUUCC-3′, wherein N represents A, U, G or C, and d vary from 18 to 60 nucleotides,
represents 4C26
SEQ ID NO: 18
5′-GGGACCUCGUUUCGGCGAUGAUGGAGAGGCGCAAGGUUAACCGCCUCAGGU
UCC(N)_{d}GGAGCCUCGCUUCGGCGAUGAUGGAGAGGCGCAAGGUUAACCGCCUC
AGGUUCC-3′, wherein N represents A, U, G or C, and d vary from 18 to 60 nucleotides,
represents 3C14
SEQ ID NO: 19
5′-GGAGCCUCGCUUAGGCGAUGAUGGAGAGGCGCAAGGUUAACCGCCUCAGGU
UCC(N)_{d}GGAACCUCGCUUCGGCGAUGAUGGAGAGGCGCAAGGUUAACCGCCUC
AGGUUCC-3′, wherein N represents A, U, G or C, and d vary from 18 to 60 nucleotides,
represents 3C22
SEQ ID NO: 20
5′-GGAACCCCGCUUCGGUGGUGAUGGAGAGGCGCAAGGUUAACCGCGUCAGGU
UCC(N)_{d}GGAACCUCGCUUCGGCGAUGAUGGAGAGGCGCAAGGUUAACCGCCUC
AGGUUCC-3′, wherein N represents A, U, G or C, and d vary from 18 to 60 nucleotides,
represents 4C6
SEQ ID NO: 21
5′-GGAACCUCGCUUCGGCGAUGAUGGAGAGGCGCAAGGUUAACCGCCUCAGGU
UCC(N)_{d}GGAAUCUCGCUUCGGCGAUGAUGGAGAGGCGCAAGGUUAACCGCCUC
AGGUUCC3-′, wherein N represents A, U, G or C, and d vary from 18 to 60 nucleotides,
represents 4C13
SEQ ID NO: 22
5′-GGAACCUCGCUUCGGCGAUGAUGGAGAGGCGCAAGGUUAACCGCCUC-GGU
UCC(N)_{d}GGAAUCUCGCUUCGGCGAUGAUGGAGAGGCGCAAGGUUAACCGCCUC
AGGUUCC-3′, wherein N represents A, U, G or C, and d vary from 18 to 60 nucleotides,
represents 3C32
SEQ ID NO: 23
5′-GGAACUUCGCUUCGGCGAUGAUGGAGAGGCGCAAGGUUAACCGCCUCAGGU
UCC(N)_{d}GGAACUUCGCUUCGGCGAUGAUGGAGAGGCGCAAGGUUAACCGCCUC-
GGUUCC-3′, wherein N represents A, U, G or C, and d vary from 18 to 60 nucleotides,
represents 3C21
SEQ ID NO: 24
5′-GGGACCUCGCUUCGGCGAUGAUGGAGAGGCACAAGGUUAACUGCCUCAGGU
UCC-3′,
represents 3C2
SEQ ID NO: 25
5′-GGAACCUCGCUUCGGCGAUGAUGGAGAGGCACAAGGUUAACUGCCUCAGGU
UCC-3′,
represents 4C2
SEQ ID NO: 26
5′-GGAACCUCGCUUCGGCGAUGAUGGAGAGGCGCAAGGUUAACCGCCUCAG-UU
CC(N)_{d}GGAACUUCGCUUCGGCGAUGAUGGAGAGGCGCAAGGUUAACCGCCUCA
GGUUCC-3′, wherein N represents A, U, G or C, and d vary from 18 to 60 nucleotides,
represents 3C3
SEQ ID NO: 27
5′-GGAACCUCGCUUCGGCGAUGAUGGAGAGGCGCAAUGUUAACCGCCUC-GGU
UCC-3′,
represents 3C31
SEQ ID NO: 28
5′-GGAACCUCGCUUCGGCGAUGAUGGAGGGGCGCAAGGUUAACCGCCUC-GGU
UCC(N)_{d}GGAACCUCGCUUCGGCGAUGAUGGAGAGGCGCAAGGUUAACCGCCUC
AGGUUCC-3′, wherein N represents A, U, G or C, and d vary from 18 to 60 nucleotides,
represents the following sequence
SEQ ID NO: 29
5′-GUGCUCGCUUCGGCAGCACAUAUACUAGUCGACUUGCCAUGUGUAUGUGGG
CCUGCAGGGGGAGACAGCUAGAGUACAGAACCCCGCUUCGGCGGUGAUGGAG
AGGCGCAAGGUUAACCGCCUCAGGUUCCGGUGACGGGGCCUCGCUUCGGCGA
UGAUGGAGAGGCGCAAGGUUAACCGCCUCAGGUUCUGACACGAGCACAGUGUA
CCCUGCAGGCCCACAUACUCUGAUGAUCCUUCGGGAUCAUUCAUGGCAAUCUA
GAGCGGACUUCGGUCCGCUUUU-3′.

Advantageously, the invention relates to the molecular complex as defined above, wherein said fluorophore is the fluorophore having one of the following the formula 6 or 7, and

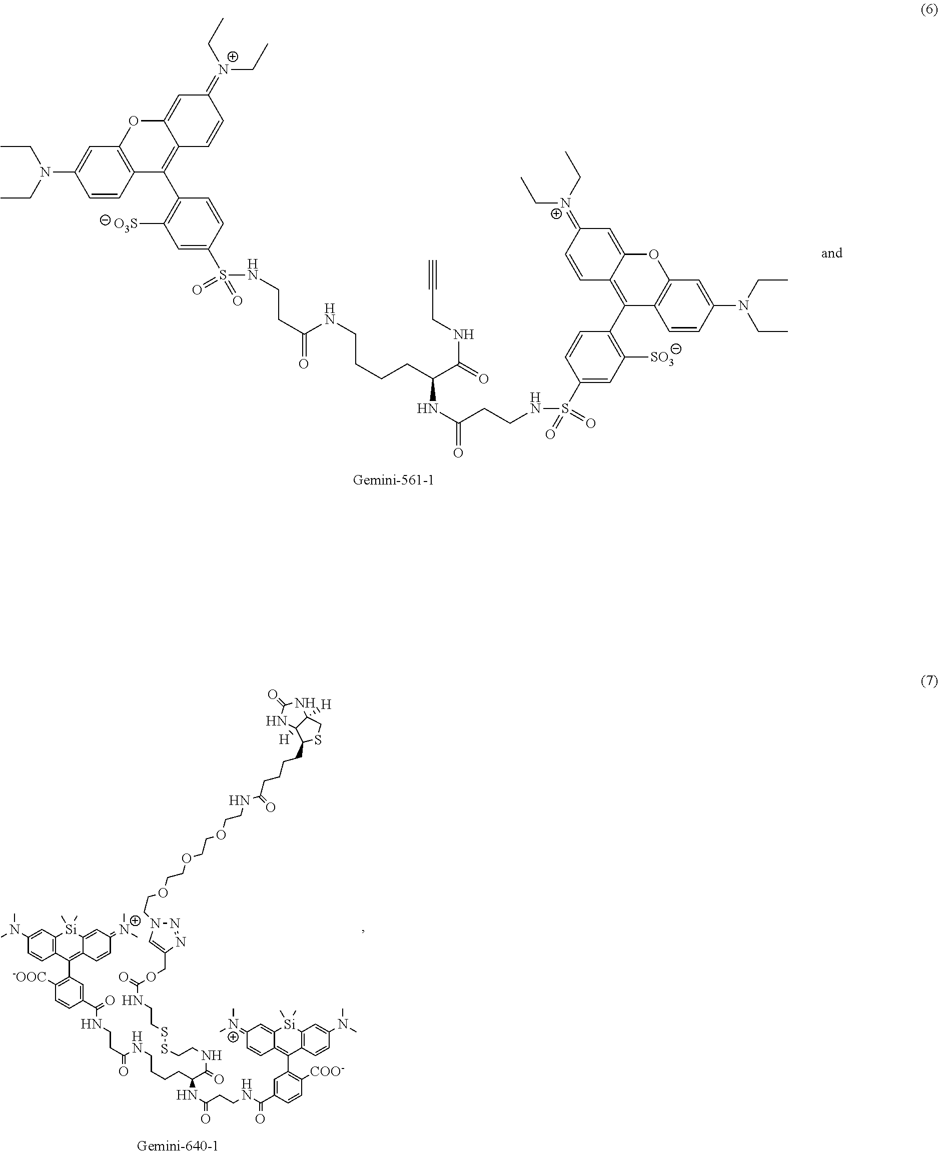

and said nucleic acid comprises, or consists essentially of, or consists of the sequence SEQ ID NO: 8 (o-Coral).

The molecular complexes according to the invention are advantageously the 5 following complexes:
compound of formula 4 and nucleic acid molecule of SEQ ID NO: 8,
compound of formula 6 and nucleic acid molecule of SEQ ID NO: 8, and
compound of formula 7 and nucleic acid molecule of SEQ ID NO: 8,

In one advantageous embodiment, the invention relates to the following complexes:
one of the compounds of formula 4, 5, 6, 7 or 8 and nucleic acid molecule of SEQ ID NO: 9,
one of the compounds of formula 4, 5, 6, 7 or 8 and nucleic acid molecule of SEQ ID NO: 10,
one of the compounds of formula 4, 5, 6, 7 or 8 and nucleic acid molecule of SEQ ID NO: 11,
one of the compounds of formula 4, 5, 6, 7 or 8 and nucleic acid molecule of SEQ ID NO: 12,
one of the compounds of formula 4, 5, 6, 7 or 8 and nucleic acid molecule of SEQ ID NO: 13,
one of the compounds of formula 4, 5, 6, 7 or 8 and nucleic acid molecule of SEQ ID NO: 14,
one of the compounds of formula 4, 5, 6, 7 or 8 and nucleic acid molecule of SEQ ID NO: 15,
one of the compounds of formula 4, 5, 6, 7 or 8 and nucleic acid molecule of SEQ ID NO: 16,
one of the compounds of formula 4, 5, 6, 7 or 8 and nucleic acid molecule of SEQ ID NO: 15,
one of the compounds of formula 4, 5, 6, 7 or 8 and nucleic acid molecule of SEQ ID NO: 18,
one of the compounds of formula 4, 5, 6, 7 or 8 and nucleic acid molecule of SEQ ID NO: 19,
one of the compounds of formula 4, 5, 6, 7 or 8 and nucleic acid molecule of SEQ ID NO: 20,
one of the compounds of formula 4, 5, 6, 7 or 8 and nucleic acid molecule of SEQ ID NO: 21,
one of the compounds of formula 4, 5, 6, 7 or 8 and nucleic acid molecule of SEQ ID NO: 22,
one of the compounds of formula 4, 5, 6, 7 or 8 and nucleic acid molecule of SEQ ID NO: 23,
one of the compounds of formula 4, 5, 6, 7 or 8 and nucleic acid molecule of SEQ ID NO: 24,
one of the compounds of formula 4, 5, 6, 7 or 8 and nucleic acid molecule of SEQ ID NO: 25,
one of the compounds of formula 4, 5, 6, 7 or 8 and nucleic acid molecule of SEQ ID NO: 26,
one of the compounds of formula 4, 5, 6, 7 or 8 and nucleic acid molecule of SEQ ID NO: 27,
one of the compounds of formula 4, 5, 6, 7 or 8 and nucleic acid molecule of SEQ ID NO: 28, and
one of the compounds of formula 4, 5, 6, 7 or 8 and nucleic acid molecule of SEQ ID NO: 29.

The invention also relates to a nucleic acid molecule comprising a first and a second region, said first and second regions being such that:
the first region comprises the nucleotide sequence of SEQ ID NO: 1; and
the second region comprises the nucleotide sequence of SEQ ID NO: 2,
   provided that said nucleic acid molecule is not the nucleic acid molecule as set forth in SEQ ID NO: 3.

Advantageously, the invention relates to the nucleic acid as defined above, wherein the nucleic acid molecule comprises, or consists essentially of, or consists of one of the nucleotide sequences as set forth in SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, and SEQ ID NO: 29.

The invention further relates to a host cell, or a non-human mammal comprising said cell, containing the nucleic acid molecule as defined above or a molecular complex as defined above, or containing a DNA molecule coding for a nucleic acid molecule as defined above, or the genetically engineered DNA molecule allowing the expression of said nucleic acid molecule, or a combination thereof.

Once the constructed DNA molecule has been cloned into an expression system, it is ready to be incorporated into a host cell. Such incorporation can be carried out by the various forms of transformation, depending upon the vector/host cell system such as transformation, transduction, conjugation, mobilization, or electroporation. The DNA sequences are cloned into the vector using standard cloning procedures in the art, as described by Maniatis et al, Cold Springs Harbor, New York (1982)), Suitable host cells include, but are not limited to, bacteria, yeast, mammalian cells, insect cells, plant cells, and the like. The host cell is preferably present either in a cell culture (ex vivo) or in a whole living organism (in vivo).
Mammalian cells suitable for carrying out the present invention include, without limitation, COS (e.g., ATCC No. CRL 1650 or 1651), BHK (e.g., ATCC No. CRL 6281), CHO (ATCC No. CCL 61), HeLa (e.g., ATCC No. CCL 2), 293 (ATCC No. 1573), CHOP, NS-1 cells, embryonic stem cells, induced pluripotent stem cells, and primary cells recovered directly from a mammalian organism. With regard to primary cells recovered from a mammalian organism, these cells can optionally be reintroduced into the mammal from which they were harvested or into other animals.

The invention relates also to the use of: the nucleic acid molecule as defined above, or the molecular complex as defined above, or the DNA molecule coding for a nucleic acid molecule as defined above, or the genetically engineered DNA molecule as defined above, or the host cell as defined above, or a mammal as defined above, or a combination thereof
for imaging, preferably in vitro or ex vivo, small molecules, RNA and proteins, preferably in cells

The target molecule of interest can be any biomaterial or small molecule including, without limitation, proteins, nucleic acids (RNA or DNA), lipids, oligosaccharides, carbohydrates, small molecules, hormones, cytokines, chemokines, cell signaling molecules, metabolites, organic molecules, and metal ions. The target molecule of interest can be one that is associated with a disease state or pathogen infection.

The invention also relates to a method for imaging in vitro or ex vivo small molecules, RNA and proteins in cells, comprising the administration to a living in vivo and ex vivo cell cultures a nucleic acid according the above definition operably linked to a biomolecule, along with a fluorophore molecule according to the above definition.

For instance, for imaging RNA in cells, it is possible to provide to a cell, or to a cell-free expression system, a molecule allowing the expression of a fusion RNA constituted by:
the RNA to be studied in the cell, operably linked, preferably in its 3′-end, but possibly to its 5′end to
an aptamer according to the above definition, or to one strand of the aptamer constituted by two separated single stranded molecules.

The above-disclosed fusion RNA is then expressed in the cell, or in a cell-free expression system, and in presence of the fluorophore, the part of the fusion molecule will interact with the fluorophore. This will result in a fluorescence emission upon exposure of an appropriate wavelength, and it could be possible to track, and thus to image, the RNA to be studied, because it is covalently linked to the aptamer.

It would be therefore possible to monitor the trafficking, the localization, the accumulation . . . of the RNA to be studied, in particular in living cells without alteration of their integrity.

It is also disclosed a method for imaging small molecules, RNA and proteins mammals, comprising the administration to a mammal a nucleic acid according the above definition operably linked to a biomolecule, along with a fluorophore molecule according to the above definition.

From conventional techniques of molecular biology, the skilled person would be able to obtain all the necessary fusion RNA molecules.

The invention will be better understood from the following figures and in light of the following examples.

### BRIEF DESCRIPTION OF THE DRAWINGS

FIG. 1: ¹H NMR spectrum of compound 1.

FIG. 2: Characterizations of Gemini-56I-Alkyne.

FIG. 2A represents the ¹H NMR spectrum of Gemini-561-Alkyne.

FIG. 2B represents the ¹³C NMR spectrum of Gemini-561-Alkyne.

FIG. 2C represents the HRMS spectrum of Gemini-561-Alkyne.

FIG. 3: Characterizations of Gemini-56I.

FIG. 3A represents the ¹H NMR spectrum of Gemini-561 (MeOD).

FIG. 3B represents the HPLC traces of Gemini-561. The signal was monitored according to ionisation detection ESI+ (top trace) and UV detection (bottom trace).

FIG. 3C represents HRMS spectrum of Gemini-S6I displaying [M+3H]⁺ and [M+2H]⁺.

FIG. 4: Design and synthesis of Gemini-561.

FIG. 4A represents the synthesis of Gemini-561

FIG. 4B represents the absorption and excitation spectra of Gemini-561 (200 nM) in water and methanol.

FIG. 4C represents the fluorescence emission spectra of Gemini-561 (200 nM) in water and methanol.

FIG. 5: Normalized absorption and emission spectra of Gemini-561 in different aqueous media mimicking cellular environments.

FIG. 6: Isolation of Gemini-561 lighting-up aptamers by in vitro evolution.

FIG. 6A represents Gemini-561 activation capacity of the parental SRB-2 and the evolved 4C10 variant. 500 nM of RNAs were incubated with 50 nM of Gemini-561 and the fluorescence was measured at À ex/em=560/600 nm.

FIG. 6B represents the monitoring of the evolution process. For each round, the enriched library was transcribed in vitro in the presence of 100 nM of Gemini-561 and the fluorescence monitored. The fluorescence apparition rate was computed for each library and normalized to that of the parental SRB-2 aptamer. The inset schematizes the different steps (A, B and C) of an evolution round. The values are the mean of 3 independent experiments, each measurement being shown as an open circle. The error bars correspond to ±1 standard deviation.

FIG. 6C is a schematic representation of genes coding for the 16 dimerized variants found among the 19 best aptamers at the end of the evolution process. For each variant, the width and the color of the box respectively inform on linker length (numerical value given on the right) and the nature of the sequence (light gray: T7 promoter, medium grey: 5′constant, dark grey: 3′ constant). Red boxes correspond to SRB-2-derived core. The clone ID refers to the round of selection from which the clone was extracted (first number) and the clone number assigned during the final screening.

FIG. 7: Gemini-561 activation by SRB-2 aptamer and its derivatives.

FIG. 7A represents a secondary structure model of SRB-2 aptamer as originally proposed. Paired regions (PI, P2 and P3) are distinguished from Loop (L2 and L3) and Junction (J2/3) regions. Constant sequence regions appended for RT-PCR amplification purposes are shown in gray.

FIG. 7A represents the fluorogenic capacity of SRB-2 and its evolved forms 4C10 and o-Coral. 500 nM of RNAS were incubated With 50 nM of Gemini-561 and the fluorescence was measured at X ex/em=560/600 nm. The values are the mean of 3 independent experiments, each measurement being shown as an open circle. The error bars correspond to ±1 standard deviation.

FIG. 8: Overall in vitro evolution strategy. Each round of evolution cycle consisted of 10 main steps. SRB-2 was used as a template for error-prone PCR (step 1) to create a DNA mutant library that was in vitro transcribed (step 2). Resulting RNAS were then selected for their binding capacity via a SELEX (Systematic Evolution of Ligands by Exponential enrichment) approach (steps 3, 4 and 5) prior to being screened for their light-up capacity using pIVC (steps 7, 8, 9 and 10). For steps performed in microfluidic chips, Oil (O) and aqueous phase (A) inlets are labeled together With inlets and outlets where Emulsion (E) were respectively reinjected and collected. Finally, the enriched pool was reamplified by an err0r-prone PCR (Step 1) before re-entering the whole process again. 4C10 was obtained after 4 rounds of this evolution cycle.

FIG. 9: Sequence and fluorogenicity of the mutants isolated upon the in vitro evolution process. Variant sequences were ordered according to their Gemini-561 activation capacity normalized to that of SRB-2 (Norm. Fluo.). Mutations are color-coded and deletions represented by a X. Structural elements are delineated by shadowed areas and paired sequences indicated under the alignment. The clone ID refers to the round of selection from which the clone was extracted (first number) and the clone number (second number) assigned during the final screening. The presence of a linker and its size are indicated between both SRB-2 derived monomers. The gene coding for each mutant was transcribed in vitro in the presence of 100 nM of Gemini-561 and the fluorescence monitored. The fluorescence apparition rate was computed for each library and normalized to that of the parental SRB-2 aptamer. It is to be noted that 5′ and 3′ constant regions are not represented. As a consequence, the numbering is downshifted by 18-nucleotides in comparison with the full-length molecule encompassing the 18-nucleotide long 5′ extension.

FIG. 10: Characterization and engineering of the evolved molecule

FIG. 10A represents the impact of linker size and 733 sequence on the capacity of 4C10 aptamer to activate Gemini-561 fluorescence. 500 nM of RNAs 734 were incubated with 50 nM of Gemini-561 and the fluorescence was measured at À ex/em=560/600. The underlined sequence corresponds to o-Coral linker.

FIG. 10B represents Contribution of the dimerization and 736 the mutations to o-Coral functionality. SRB-2 aptamer was used as scaffold either in its monomeric 737 (m) or dimeric (d) form containing o-Coral linker. Indicated mutations were then implemented and the 738 different constructs tested as above.

FIG. 10C represents the identification of interacting regions. A destabilized mutant (₆₇GGUUC₇₁/₆₇CCAAG₇₁) of o-Coral and two potential compensatory mutants (1: ₆₇GGUUC₇₁/₆₇CCAAG_{71_20}GAACC_{24/20}CUUGG₂₄ and 2: ₆₇GGUUC₇₁/₆₇CCAAG_{71_79}GGGCC_{85/79}CUUGG₈₅) were prepared and tested as above. The values (a-c) are the mean of 3 independent experiments, each measurement being shown as an open circle. The error bars correspond to ±1 standard deviation.

FIG. 10D represents the fluorescence emission spectra of Gemini-561 (200 nM) in absence and in the presence of RNA aptamers (600 nM). Excitation wavelength was 530 nm.

FIG. 10E represents the Spectral and biochemical properties of Gemini-561 alone or in complex with SRB-2 or o-Coral aptamers. Measures were performed in selection buffer (40 mM phosphate buffer pH7.5, 100 mM KCl, 1 mM MgCl₂ and 0.05% Tween-20).

FIG. 10F represents Model of secondary structure for o-Coral aptamer. This model was established based on enzymatic probing experiments (FIGS. 12A and B) and mutagenesis experiments shown on c. SRB-2 derived sequences (Part A and B) are shown in black or red whereas the constant regions and the linker are shown in grey. Acquired mutations found to contribute to o-Coral function are circled in black.

FIG. 11: Refinement of structural model using P1 compensatory mutants.

FIG. 11A represents three mutants that were generated: a destabilized mutant (Destabilized stem: ₆₇GGUUC₇₁/₆₇CCAAG₇₁) of o-Coral and two potentially compensatory mutants; the first one based on the independent folding model (₆₇GGUUC₇₁/₆₇CCAAG_{71_20}GAACC₂₄/₂₀CUUGG₂₄) and the second one based on the intertwined folding model (₆₇GGUUC₇₁/₆₇CCAAG_{71_79}GGGCC₈₅/₇₉CUUGG₈₅). SRB-2 derived sequences (Part A and B) are shown in black or red whereas the linker sequence is shown in grey. Implemented mutations described before are shown.

FIG. 11B represents Impact of implemented mutations on o-Coral aptamer fluorogenicity. 500 nM of RNAS were incubated With 50 nM of Gemini-561 and the fluorescence was measured at λ ex/em=560/600 nm. The values are the mean of 3 independent experiments, each measurement being shown as an open circle. The error bars correspond to ±1 standard deviation.

FIG. 12: Refinement of structural model using P1 compensatory mutants.

FIG. 12A is representation of o-Coral and the ₆₉UU₇₀/₆₉CC₇₀ o-Coral double mutant according to the independent folding model (upper part) and the intertwined folding model (lower part). SRB-2 derived sequences (Part A and B) are shown in black or red whereas the linker sequence is shown in grey. Implemented mutations described below is shown in orange.

FIG. 12A Impact of the implemented mutation on o-Coral aptamer fluorogenicity. 500 nM of RNAs were incubated with 50 nM of Gemini-561 and the fluorescence was measured at λ ex/em=560/600 nm. The values are the mean of 3 independent experiments, each measurement being shown as an open circle. The error bars correspond to ±1 standard deviation.

FIG. 13: Probing of o-Coral secondary structure.
(a) Probing experiment. Radioactively labelled o-Coral RNA was subjected to digestion by V1, T1 or T2 nucleases prior to analyzing the digestion products on 10% polyacrylamide denaturing gels. The increased concentration of the enzymes is schematized by the colored triangles (V1: 0.001 U/μL-0.002 U/μL-0.004 U/μL, TI: 0.25 U/μL-0.5 U/μL-1 U/ptL, T2: 0.0125 U/μL-0.025 U/μL-0.05 U/μL). Ctrl lane corresponds to an enzyme-free experiment, AH stands for Alkaline Hydrolysis in which o-Coral was statistically hydrolyzed, dT1 stands for denaturing T1 cleavages. The numbers on the right refer to o-Coral nucleotides.
(b) Secondary structure model of o-Coral. TI, T2 and V1 cleavage sites are indicated respectively by the blue, green and red arrows. SRB-2 derived monomers are shown in black or red (Part A and B), whereas constant regions and linker are shown in gray. Acquired mutations found to contribute to o-Coral function are circled in black.

FIG. 14: Secondary structure models of o-Coral aptamer.

FIG. 14A represents an independent folding model. In this model, each SRB-2-derived monomers adopts an independent folding and closely resemble the original SRB-2 molecule associated by single stranded linker region.

FIG. 14A represents an Intertwined folding model. In this model, both each SRB-2 derived monomers fold on each other and form an intertwined structure. On both models, SRB-2 derived monomers are shown in black or gray (Part A and B), whereas constant regions and linker are shown in light gray. Acquired mutations found to contribute to o-Coral function are shown are circled in black.

FIG. 15: Salt dependency of Gemini561/o-Coral module.

FIG. 15A is a graph that represents the monovalent ions dependency of o-Coral. O-Coral RNA and Gemini-561 were mixed in a solution containing 40 mM Phosphate buffer pH7.5, 100 mM KCl or NaCl or CsCl or LiCl or in the absence of monovalent cations, 2 mM MgCl₂ and 0.05% Tween-20 and the fluorogenic capacity was measured.

FIG. 15B is a graph that represents magnesium dependency of o-Coral. O-Coral RNA and Gemini-561 were mixed in a solution containing 40 mM Phosphate buffer pH7.5, 100 mM KCl, the indicated concentration of MgCl₂ and 0.05% Tween-20 and the fluorogenic capacity was measured. For both condition (a and b), 500 nM of RNAs were incubated with 50 nM of Gemini-56I and the fluorescence was measured at λ ex/em=560/600 nm. The values are the mean of 3 independent experiments, each measurement being shown as an open circle. The error bars correspond to ±1 standard deviation.

FIG. 16: Left: Normalized absorption and Right: emission spectra of Gemini-561 (200 nM) in absence and in the presence of RNA aptamers (600 nM). Excitation wavelength was 530 nm.

FIG. 17: Absorption and emission spectra of Gemini-561 (200 nM) in the presence of increasing concentrations (equivalents, eq.) of o-Coral. Excitation wavelength was 530 nm.

FIG. 18: (a) Absorption and (b) emission spectra of Gemini-561 (200 nM) in the presence of increasing concentration (equivalents, eq.) of SRB-2 aptamer. Excitation wavelength was 530 nm. Normalized spectra of (c) absorption and (d) emission spectra respectively.

FIG. 19 is a graph showing the effect of biomolecules and biological medium on the fluorescence intensity of Gemini-561/o-Coral (1/1 molar ratio) complex at 0.2 μM concentration. After Gemini-561/o-Coral complex was formed, the mixture was incubated with the corresponding biomolecule (BSA 10 mg/mL, non-targeted DNA 50 μM or SRB-2 aptamer 0.2 μM) or biological medium (FBS 10%) for 15 min and the fluorescence was recorded at 596 nm. Excitation wavelength was 530 nm. The values are the mean of 3 independent experiments, each measurement being shown as a colored dot. The error bars correspond to ±1 standard deviation.

FIG. 20: Cytotoxicity assay of Gemini-561. HeLa cells were incubated with various concentration of Gemini-561 and their viability was assessed after 24 hours using MTT test. An incubation with 0.1% Triton X100 was used as positive control. The values are the mean of 3 independent experiments, each measurement being shown as a colored dot. The error bars correspond to ±1 standard deviation.

FIG. 21: Microinjection in HeLa cells.

FIG. 21A: Microinjection of Gemini-561 (1 μM) alone (in cytosol), complex of Gemini-561/o-Coral (1 μM) or Gemini-561/SRB-2 (1 μM) (in cytosol). Arrows show that Gemini-561/o-Coral complex was microinjected into either nucleus or cytosol. Scale bar is 20 μm.

FIG. 21B: Microinjection of o-Coral (52 μM) or SRB-2 (52 μM) with Dextran-Alexa-647 conjugate (10 μM) in cells pre-treated with Gemini-561 (200 nM) for 5 min. Microinjection parameters: Pi=90 [hPa]; Ti=0.3 [s]; Pc=10 [hPa]. The nucleus was stained With Hoechst (5 μg/mL). The images were acquired using a 10 s exposure time. Gemini-561 (ex: 550 nm, em: 595±40 nm), Hoechst (ex: 395 nm, em: 510±42 nm) and Alexa-647 (ex: 638 nm, em: 810±90 nm). Scale bar is 30 μm.

FIG. 22: Live-cell imaging of o-Coral expressed from pol. II and pol. Ill promoter. Live cell imaging of HeLa (FIG. 22A) and HEK293T (FIG. 22B) cells expressing o-Coral from the U6—promoter, the gfp mRNA labelled With single copy of o-Coral in the 3′untranslated region (3′ UTR) or eGFP only. Cells were incubated with Gemini-56I (200 nM) for 5 min before imaging. Hoechst was used to stain the nucleus (5 μg/mL). The images were acquired using a 500 ms exposure time. Gemini-561 in red (ex: 550 nm, em: 595140 nm), Hoechst in blue (ex: 395 nm, em: 510142 nm) and eGFP in green (ex: 470 nm, em: 53 1140 nm). Scale bar is 30 μm.

FIG. 23: Live-cell imaging of o-Coral expressed from pol. II and pol. III promoter.

FIG. 23A: Live cell imaging of HeLa cells expressing o-Coral from the U6-promoter in the absence and presence of Actinomycin D, cells expressing eGFP only and untransfected (untr.) cells treated with Actinomycin D.

FIG. 23B: Live cell imaging of HeLa cells expressing the gfp mRNA labelled with single copy of o-Coral. Cells expressing the gfp mRNA with or without scaffold inserted and untransfected cells were used as negative controls. Cells were incubated With Gemini-561 (200 nM) for 5 min before imaging. Hoechst was used to stain the nucleus (5 μg/mL). The images were acquired using a 500 ms exposure time. Gemini-561 in red (ex: 550 nm, em: 595140 nm), Hoechst in blue (ex: 395 nm, em: 5 10±42 11 m) and eGFP in green (ex: 470 nm, em: 531±40 nm). Scale bar is 30 μm.

FIG. 24: Live-cell imaging of transfected HeLa cells expressing eGFP and o-Coral, eGFP only or eGFP and F30 scaffold only. Top panel shows Gemini-56I channel only. Bottom channel shows merged all channels. Cells were incubated with Gemini-56I (200 nM) for 5 min. White arrows on the images depict the correlation between expression of eGFP and o-Coral as well as the different transcription states of cells. The images were acquired using a 500 ms exposure time. Gemini-561 in red (ex: 550 nm, em: 595±40 nm), Hoechst in blue (ex: 395 nm, em: 510±42 nm) and eGFP in green (ex: 470 nm, em: 531±40 nm). Scale bar is 30 μm.

FIG. 25 Comparative analysis of photostability by fluorescence microscopy and spectroscopy.

FIG. 25A: Photostability measurement in live Hela cells. In vitro transcribed and purified aptamers were preincubated With respective fluorogenic dyes for 10 min in selection buffer to form complex. Complexes were microinjected in live HeLa cells using 5 μM dye and 20 μM aptamer concentration. Microinjection parameters: Pi=90 [hPa]; Ti=0.3 [s]; Pc=10 [hPa]. Consecutive images were acquired, each using a 500 ms exposure time. The excitation power was adjusted for the fluoromodules to absorb similar amount of photons. Broccoli (ex: 470 nm, em: 475±50 nm); Corn (ex: 470 nm, em: 531±40 nm); Mango (ex: 470 nm, em: 531±40 nm); Coral (ex: 550 nm, em: 595±40 nm). Scale bar is 30 ptm.

FIG. 25B: Fluorescence intensity decay curves over the time. Data represent average values ±1 S.D. extracted from images from 3 independent experiments (n=3).

FIG. 25 C: Signal to background noise ratio of the first acquired image from a depicting the brightness of the system and the quality of obtain images. Signal to background noise ratios were calculated from fluorescence intensity values extracted from images using same region of interest from 3 independent injections. The value of each measurement is shown as a colored dot. The error bars correspond to ±1 standard deviation.

FIG. 25D: Photostability of G561/o-Coral (0.2 μM/1 μM) compared to Broccoli+DFHBI-1T (0.2 μM/1 μM), Corn+DFHO (0.2 μM/1 μM), Mango+TO1-Biotin (0.2 μM/1 μM). Each complex was excited at the same molar extinction coefficient value; 30,000 M⁻¹ cm⁻¹ Broccoli, Corn and Mango were excited using 488 nm laser (7.75 mW cm⁻², 11 mW cm⁻², 10 mW cm⁻² respectively) and o-Coral was excited using 532 nm laser (7 mW cm⁻²). Fluorescence intensity was monitored at 507 nm for Broccoli, 545 nm for Coin, 535 nm for Mango and 596 nm for o-Coral.

FIG. 26: Photostability of aptamer-dye couples in live HeLa cells. Broccoli/DFHBI-1T, Corn/DFHO, Mango/TO1-biotin and o-Coral/Gemini-561 photostability was assessed by fluorescence microscopy. Cells were preincubated with corresponding dye (10 μM DFHBI-1T, 10 μM DFHO, 0.2 μM TO1-biotin for 30 min and 0.2 μM Gemini-561 for 5 min). Aptamers were microinjected in live HeLa cells at 20 μM concentration. Microinjection parameters: Pi=90 [hPa]; Ti=0.3 [S]; Pc=IO [hPa]. Consecutive images were acquired, each using a 500-ms exposure time. Broccoli (ex: 470 nm, em: 475±50 nm); Corn (ex: 470 nm, em: 531±40 nm); Mango (ex: 470 nm, em: 531±40 nm); Coral (ex: 550 nm, em: 595±40 nm). The excitation power was adjusted to reach similar emission intensity. Fluorescence intensity values were extracted using same region of interest from 3 independent injections. The values are mean±S.D (n=3).

FIG. 27: Photostability of Gemini-561/o-Coral over extensive constant illumination. A mixture of Gemini-561/o-Coral (1 μM/2 μM) was prepared and individualized into water-in-oil droplets to prevent unwanted exchange of complexes between illuminated and non-illuminated areas as described before. The emulsion was then exposed to a constant illumination wavelength (575 nm) at the maximum intensity of the light source (Spectra X, Lumencor), and the emitted fluorescence (625±50 nm) was collected by an Orca-Flash IV camera for 500 ms every 100 ms with ×40 objective (numerical aperture (NA) 0.45). The values are the mean of 3 independent experiments and the error bars correspond to ±1 standard deviation.

FIG. 28: Normalized absorption spectra of Gemini-552 (200 nM) in absence (black line) and in the presence of o-Coral (dashed line) or SRB-2 (dotted line) RNA aptamers (600 nM) in an aqueous buffer (pH 7.4).

FIG. 29: Fluorescence emission spectra of Gemini-552 (200 nM) in absence (black line) and in the presence of o-Coral (dashed line) or SRB-2 (dotted line) RNA aptamers (600 nM) in an aqueous buffer (pH 7.4). Excitation wavelength was 520 nm.

FIG. 30: In vitro transcription monitoring of SIR-A in the presence of Gemini 640-2. An in vitro transcription mixture containing Gemini 640-2 was supplemented (gray circles) or not (gray triangle) with DNA coding for SIR-A aptamer and the red fluorescence (ex.=640 nm/em.=680 nm) was monitored over the time at 37° C.

FIG. 31: An in vitro transcription mixture containing 100 nM Gemini 640-2 was supplemented with o-Coral-coding DNA (dashed bars), SIRA-coding DNA (dotted bars) or without DNA (open bar), and the red fluorescence (ex.=640 nm/em.=680 nm) was monitored over the time at 37° C. The measured fluorescence was then normalized to that of the reaction without DNA (H20).

FIG. 32: An in vitro transcription mixture containing 100 nM Gemini 561-2 was supplemented with o-Coral-coding DNA (dashed bars), SIRA-coding DNA (dotted bars) or without DNA (open bar), and the orange fluorescence (ex.=560 nm/em.=600 nm) was monitored over the time at 37° C. The measured fluorescence was then normalized to that of the reaction without DNA (H20).

### EXAMPLES

### Example 1

In this example, the inventors propose a new concept for preparation of bright and photostable fluorogen for RNA imaging in cells by exploiting dimerization-induced self-quenching of SRB dyes, which yielded fluorogen Gemini-561. Following a new selection scheme combining SELEX and pIVC, together with molecular engineering, they developed o-Coral, a light-up aptamer of unprecedented compact dimeric structure, able to form a high affinity, bright and photostable complex with Gemini-561. This set of unique features allows live-cell imaging mRNAs labelled with a single copy of the o-Coral aptamer.

### Methods

### Synthesis of Gemini-561

All starting materials for synthesis were purchased from Alfa Aesar, Sigma Aldrich or TCI Europe and used as received unless stated otherwise. NMR spectra were recorded on a Bruker Avance III 400 MHz spectrometer. Mass spectra were obtained using an Agilent Q-TOF 6520 mass spectrometer.

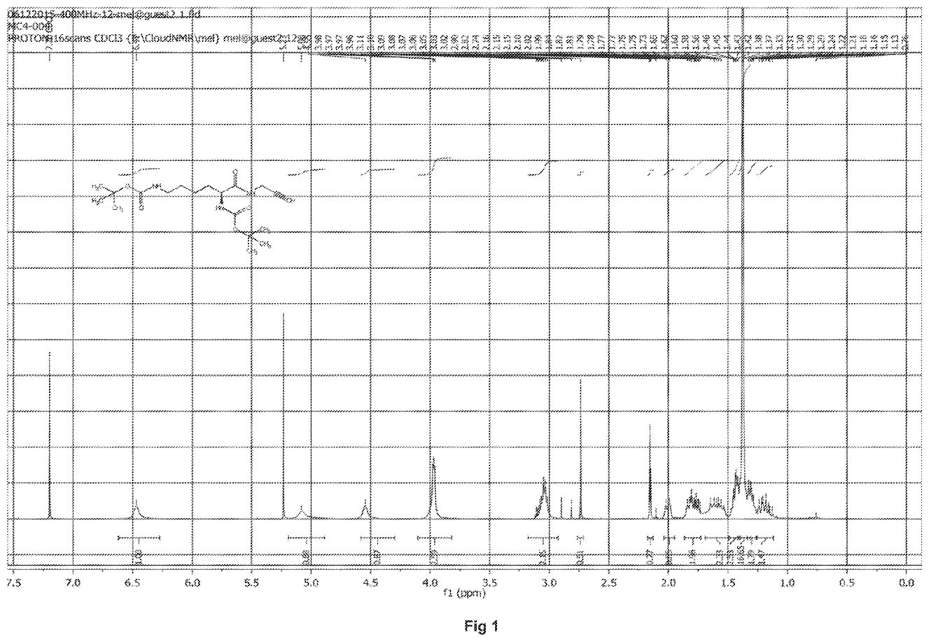

Compound 1. To a mixture of di-boc-L-lysine dicyclohexylamine salt (1.53 g, 2.89 mmol) and propargylamine (277 μL, 4.33 mmol, 1.5 eq) in dry DMF (20 mL) was added HATU (1.32 g, 3.46 mmol, 1.2 eq) followed by DIEA (1.5 mL, 8.67 mmol, 3 eq). After 1 h, the solvents were evaporated and the product was extracted with EtOAc and washed with water (2 times) and brine. The organic phase was fried over anhydrous MgSO₄, filtered and evaporated. The crude was purified by column chromatography on silica gel (DCM/MeOH: 98/2) to obtain 822 mg of 1 (74% yield) as a white solid. The NMR (FIG. 1) was in accordance with the literature.

Compound 2. 2 was obtained following a protocol described in Hatai, J, et al. J Am Chem Soc 139, 2136-2139 (2017)).

Gemini-561-alkyne. To a solution of 1 (650 mg, 1.69 mmol) in DCM (20 mL) were added 5 mL of TFA. The reaction was allowed to stir at room temperature and after 1 h the solvents were evaporated, the crude was dissolved in a minimum of MeOH and the product was precipitated in ether. The deprotected product in form of oil was used in the next step without further purification.

In a separate flask, to a solution of 2 (50 mg, 0.073 mmol) in DCM (2 mL) were added 2 mL of TFA. After 2 h, the solvents were evaporated and the deprotected carboxylic acid was involved in the next step without further purification.

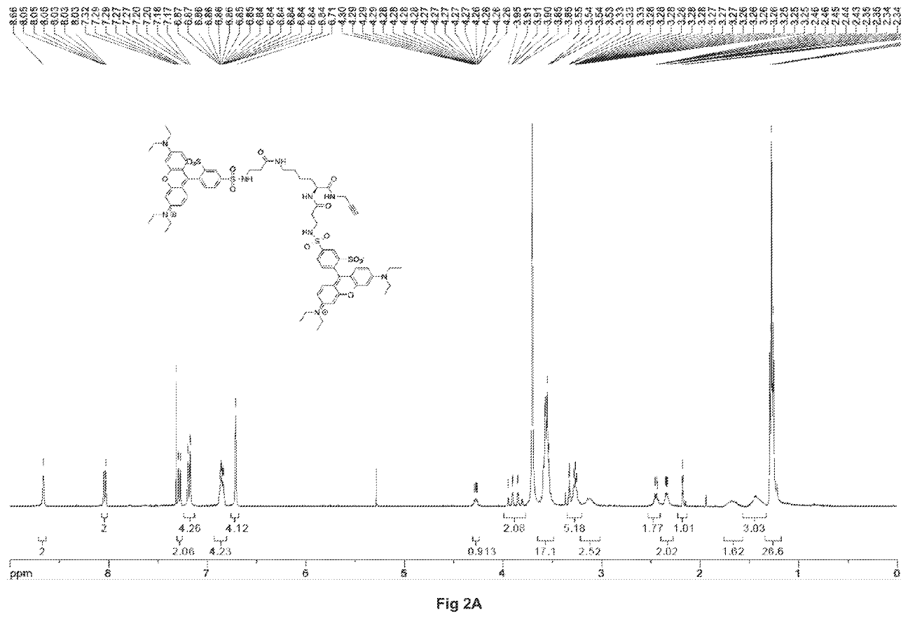

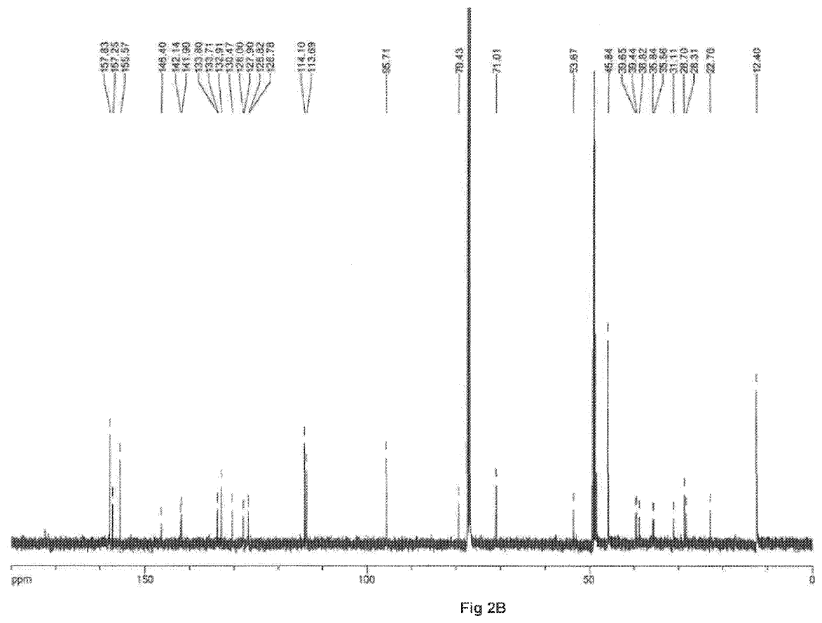

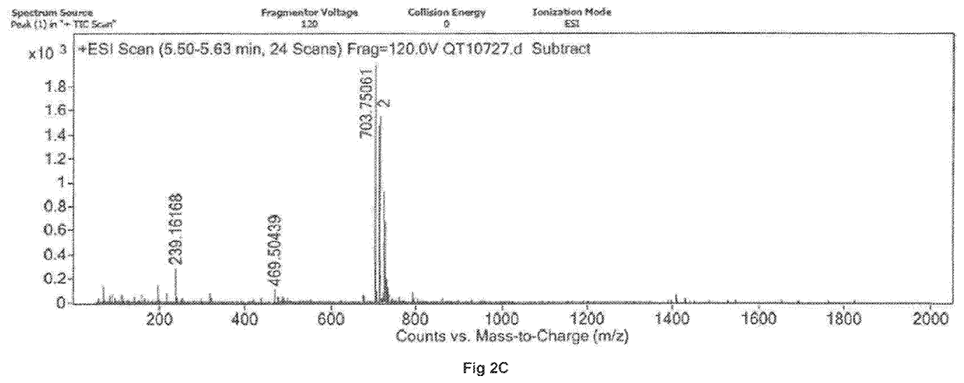

To a solution of deprotected 2 (0.073 mmol, 2 eq) and deprotected 1 (15 mg, 0.037 mmol, 1 eq) in DMF (3 mL) was added HATU (17 mg, 0.044 mmol, 1.2 eq) followed by DIEA (20 μL, 0,111 mmol, 3 eq). After 1 h the solvents were evaporated and the crude was first purified by column chromatography on silica gel (DCM/MeOH: 8/2) and was further purified by reverse phase column chromatography (C-18 column, ACN/Water: 20/80 to 100/0 over 30 minutes) to obtain 25 mg g of Gemini-561-alkyne (48% yield) as dark violet syrup. Rf=0.27 (DCM/MeOH: 9/1). ¹H-NMR (400 MHz, CHCl₃/MeOD): δ 8.66 (s, 2H, H Ar), 8.04 (dt, J=8.0, 1.6 Hz, 2H, H Ar), 7.28 (dd, J=8.0, 0.7 Hz, 2H, H Ar), 7.19 (dd, J=9.5, 2.7 Hz, 4H, H Ar), 6.87-6.84 (m, 4H, H Ar), 6.71 (s, 4H, H Ar), 4.30-4.26 (m, 1H, Ha lysine), 3.95-3.85 (m, 2H, CH₂), 3.55-3.53 (m, 16H, CH₂; Et), 3.33-3.25 (m, 8H, 4 CH₂), 2.46-2.43 (m, 2H), 2.35-2.33 (m, 2H), 2.18 (t, J=2.4 Hz, 1H, C≡CH), 1.45-1.41 (m, 4H, 2 CH₂), 1.28 (t, J=7.1 Hz, 24H, CH₃Et). ¹³C-NMR (126 MHz, CHCl₃/MeOD): Note that some peaks are doubled due to rotamers formed with amide bonds. ¹³C-NMR (126 MHz, CHCl₃/MeOD): δ 157.8 (C Ar), 157.2 (C Ar), 155.5 (C Ar), 146.4 (C Ar), 142.1 (C Ar), 141.9 (C Ar), 133.8 (C Ar), 133.7 (C Ar), 132.9 (C Ar), 130.4 (C Ar), 128.0 (C Ar), 127.9 (C Ar), 126.8 (C Ar), 126.7 (C Ar), 114.1 (C Ar), 113.6 (C Ar), 95.7 (C Ar), 79.4 (C alkyne), 71.0 (C alkyne), 53.6 (Ca), 45.8 (CH₂Et), 39.6, 39.4, 38.8, 35.8, 35.5, 31.1, 28.7, 28.3, 22.7, 12.4 (CH₃Et). HRMS (ESI+), calcd for C₆₉H₈₅N₉O₁₅S₄ [M+2H]⁺ 703.7518. found 703.7506 (FIGS. 2A-C).

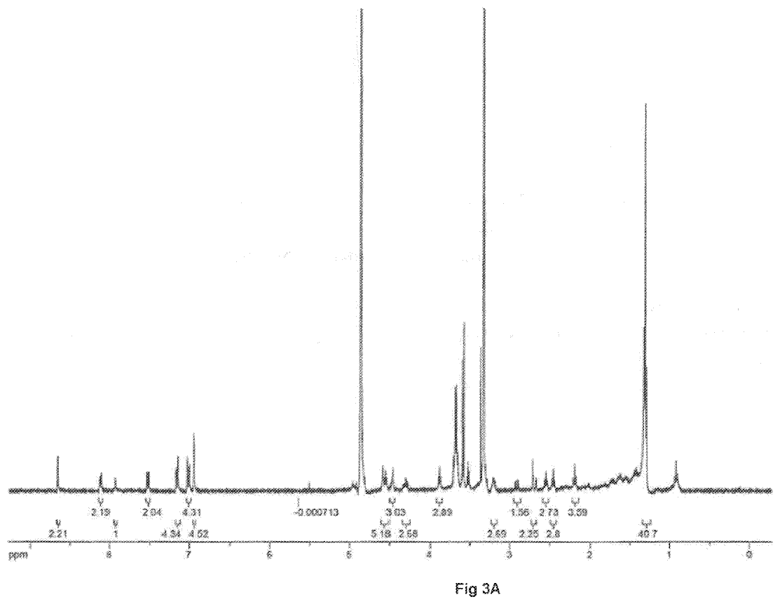

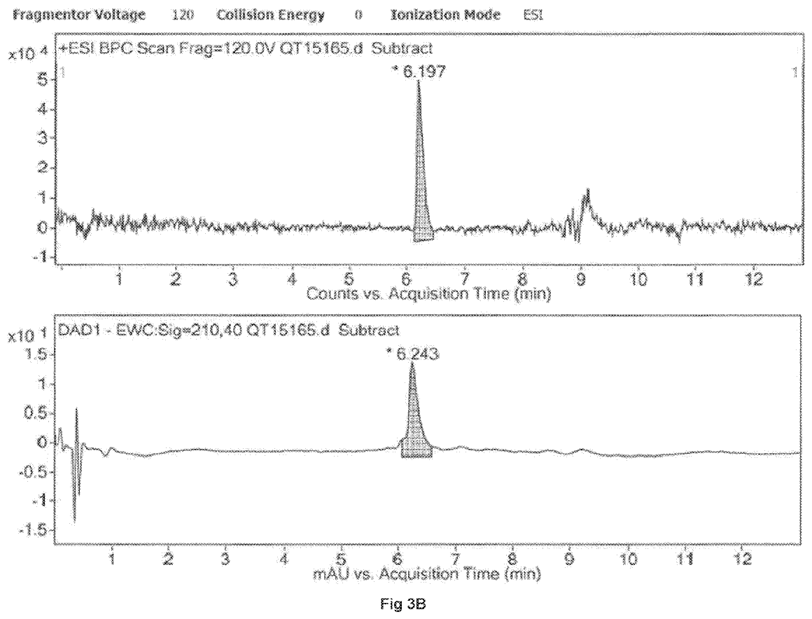

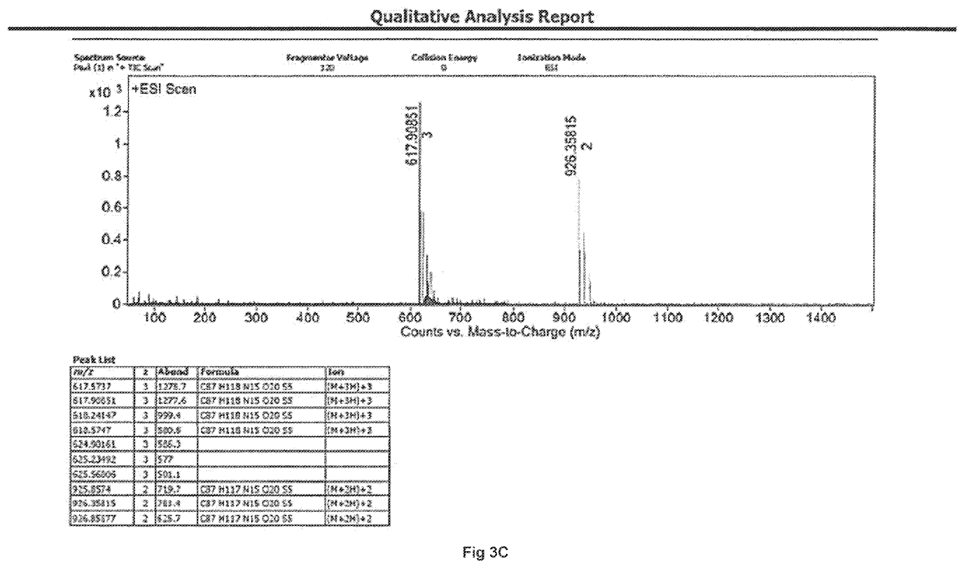

Gemini-561. To a solution of Gemini-561-alkyne (25 mg, 17.7 μmol) and biotin-PEG₃-N3 (12 mg, 26.6 μmol, 1.5 eq) in DMF (2 mL) was added a mixture of CuSO₄·5H₂O (3 mg) and sodium ascorbate (3 mg) in water (0.2 mL). The solution was allowed to stir at 60° C. overnight. The solvents were evaporated and the product was purified by preparative TLC using DCM/MeOH (85/15) as eluent to obtain 16 mg of Gemini-561 (49% Yield) as dark violet syrup. Rf=0.23 (DCM/MeOH: 85/15). ¹H-NMR (400 MHz, MeOD): δ 8.66-8.64 (m, 2H, H Ar), 8.14-8.10 (m, 2H, H Ar), 7.94-7.91 (m, ¹H, H triazol), 753-750 (m, 2H, H Ar), 7.17-7.13 (m, 4H, H Ar), 7.03-6.99 (m, 4H, H Ar), 6.96-6.92 (m, 4H, H Ar). The rest of the spectrum is difficult to assign probably due to rotamers and the weak amount of product. HPLC traces are provided as a proof of the purity of Gemini-561 HPLC: Zorbax SB-CI8, particle size 1.8 μm (Agilent), ACN/Water (0.05% formic acid) 2/98 to 100/0 in 8 min, 0.5 mL/min. HRMS (ESI+), calcd for Ca₇H₁₁₈N₁₅O₂₀S₅[M+3H]⁺ 617.5755. found 617.5737 (FIGS. 3A-C).

### Optical Spectroscopy

The water used for optical spectroscopy was Milli-Q water (Millipore®). All the solvents were spectroscopy grade. Absorption and emission spectra were recorded on a Gary 4000 Scan ultraviolet-visible spectrophotometer (Varian) and a FluoroMax-4 spectrofluorometer (Horiba Jobin Yvon) equipped with a thermostated cell compartment, respectively. For standard recording of fluorescence spectra, the emission was collected 10 nm after the excitation wavelength. All the spectra were corrected from wavelength-dependent response of the detector and measured at room temperature (25° C.). Absorbance values of all solutions were systematically below 0.1 at the maximum. Quantum yields were determined using a reference dye (Rhodamine B in water).

### Gene Library Generation

The sequence coding for the SRB aptamer was flanked with constant regions at 5′ (GGGAGACAGCTAGAGTAC—SEQ ID NO: 30) and 3′ end (GACACGAGCACAGTGTAC—SEQ ID NO: 31) to allow DNA amplification and RNA reverse transcription. Mutant libraries were generated by error prone polymerase chain reaction (PCR) by subjecting 10 fmoles of DNA (diluted in 200 yg/ml Of yeast total RNA solution (Ambion)) to 4 amplification cycles in the presence of Fwd (CTTTAATACGACTCACTATAGGGAGACAGCTAGAGTAC—SEQ ID NO: 32) and Rev (GACACGAGCACAGTGTAC—SEQ ID NO: 33) primers as well as nucleotide analogues (JBS dNTPMutagenesis Kit, Jena Bioscienoe) as described before”. 1 ng of PCR products was amplified in a second PCR mixture containing 10 pmoles of each primer (Fwd and Rev), 0.2 mM of each dNTPS, 5 U of Phire II® (Fermentas) and the corresponding buffer (Fermentas). The mixture was thermocycled starting with an initial step of denaturation of 30 sec at 95° C. followed by 25 cycles of: 5 sec at 95° C. and 30 sec at 60° C. The PCR products were purified following the “Wizard® SV Gel and PCR Clean-up System” (Promega) kit instructions and the quantity of DNA was determined by NanoDrop measurement.

### In Vitro Transcription and RNA Purification

Genes coding for aptamers were PCR amplified with the same procedure used before (25 cycles of PCR using Phirell enzyme). 1 μg of PCR products was then in vitro transcribed in 500 μl of mixture containing 2 mM of each NTP (Larova), 25 mM MgCl2, 44 mM Tris-HCl pH 8.0 (at 25° C.), 5 mM DTP, 1 mM Spermidine and 17.5 μg/ml T7 RNA polymerase (prepared in the laboratory). After 3 h of incubation at 37° C., 1000 units of Baseline-Zero™ DNase (Epicentre) and the corresponding buffer were added to the mixture and a second incubation of 1 h at 37° C. was performed. RNAs were then recovered by phenol extraction. In vitro transcribed RNA was then purified using ion exchange chromatography (FastFlow DEAE sepharose, GE Healthcare) by loading the RNA in and washing the resin with bind/wash buffer (50 mM NaCl, 50 mM Tris-HCl pH 7.5 and 10% Glycerol) and eluting it using elution buffer (600 mM NaCl and 50 mM Tris-HCl pH 7.5). Alternatively, RNA was gel purified by ethanol precipitating transcription mixture and dissolving the pellet into loading buffer (0.05% bromophenol blue, 20% glycerol, TBE 1×, 8M urea). The solution was then loaded on a 12% denaturing 8 M urea acrylamide/bisaarylamide gel. The piece of gel containing RNA was identified by UV shadowing, and the RNA electroeluted as described before. Eluted RNA was then ethanol precipitated, the washed pellets were dissolved in DEPC treated water and quantified With Nanodrop (Thermo Scientific).

### SELEX

100 μL of streptavidin-agarose beads (Sigma-Aldrich) were washed with 200 μL of activation buffer (100 mM NaOH, 50 mM NaCl). The beads were then centrifuged 5 minutes at 5000 g and room temperature, then the supernatant was removed by pipetting. This procedure was repeated with 200 μL of pre-wash buffer (40 mM potassium phosphate buffer pH 7.5, 100 mM KCl, 1 mM MgCl2; and 0.05% Tween 20) and finally 200 μL of wash buffer (pre-wash buffer supplemented with 1 mg/mL BSA (New England Biolabs), 0.1 mg/mL sodium heparin (Sigma-Aldrich) and 200 μg/mL yeast total RNA (Ambion). The resin was loaded into a cartridge (Plastic small column CS-20 ABT) previously equilibrated by an overnight incubation with wash buffer at 4° C. Then, 500 μL of wash buffer supplemented with 10 nmoles of biotinylated Gemini-561 dye were added on the beads at a controlled flow-rate of 10 mL/h using a syringe pump (PhD 2000, Harvard Apparatus). Afterward, the unbound fluorophore was washed away by 20 mL of wash buffer (20 mL/h). About 50 μg of purified RNA were introduced in 250 μL of ultra-pure DEPC-treated water and renatured by 2 min at 85° C. followed by 5 min at 25° C. Then, 250 μL of twice concentrated washing buffer were added and the mixture was infused through the Gemini-561 substituted resin at a flow-rate of 1.5 mL/h. Unbound RNAS were eliminated per 20 mL of wash buffer infused at 20 mL/h. This initial wash was followed by three additional washes by 15 mL of buffer of while reducing the ionic strength (respectively 100 mM, 10 mM and 1 mM KCl). The selection pressure was further increased during the last round by introducing 5 μM of free Gemini-561 dye during the last wash of the column. The beads were then collected using a Pasteur pipette, centrifuged and placed in 100 μL of elution buffer (95% formamide and 25 mM EDTA). After 2 minutes of heating at 90° C., the beads were centrifuged, the supernatant was recovered, and the RNA precipitated as above. RNA was pelleted, washed and resuspended in 50 μL of 2 μM Rev primer solution. The mixture was heated for 2 min at 85° C. and cooled at 25° C. for 5 min. 50 μL of reaction mixture containing 0.5 mM of each dNTP, 400 U of reverse transcriptase (Maxima H Minus, ThermoFisher) and the corresponding 2× concentrated buffer were added and the mixture and incubated 1h00 at 55° C. The resulting cDNA was then extracted with a mixture Phenol/Chloroform/Isoamyl alcohol 25/24/1 (Roth) and precipitated. cDNA was recovered by 30 minutes of centrifugation at 21000 g and 4° C., washed, dried, resuspended in 250 μL of Phirell PCR mixture and amplified by PCR as described above.

### Droplet-Based Microfluidics

Microfluidic chips were made of polydimethylsiloxane (PDMS) as described in Ryckelynck, M. et al. RNA 21, 458-69 (2015).

i. Droplet digital PCR. DNA mutant libraries were diluted in 200 μg/mL yeast total RNA solution (Ambion) down to ˜8 template DNA molecules per picoliter. 1 μL of this dilution was then introduced in 100 μL of PCR mixture containing 0.2 μM of each primer (Fwd and Rev), 0.2 mM of each dNTPs, 20 μM of coumarin, 0.1% Pluronic F68 (Sigma), 5 U of Phire II DNA polymerase enzyme (Fermentas) and the corresponding buffer (Fermentas). The mixture was loaded in a length of PTFE tubing and infused into droplet generator microfluidic chip where it was dispersed in 2.5 pL droplets (production rate of about 12 000 droplets/s) carried by HFE 7500 fluorinated oil (3M) supplemented with 3% of a fluorosurfactant (proprietary synthesis). Droplet production frequency was monitored in real time using an optical device and software developed by the authors of Ryckelynck, M. et al. RNA 21, 458-69 (2015) and used to determined droplet volume. 2.5 μL droplets were generated by adjusting pumps flowrates (MFCS, Fluigent). The emulsion was collected in 0.2 mL tubes and subjected to an initial denaturation step of 2 min at 98° C. followed by 30 cycles of: 10 sec at 98° C., 30 sec at 55° C., 30 sec at 72° C. Droplets were then reinjected into a droplet fusion microfluidic device.

ii. Droplet fusion. PCR droplets were reinjected and spaced into a fusion device at a rate of 4500 droplets/s. Each PCR droplet was then synchronized with a 16 μL in vitro transcription (IVT) droplet containing 2 mM each NTP (Larova), 25 mM MgCl, 44 mM Tris-HCl pH 8.0 (at 25° C.), 5 mM DTT, 1 mM Spermidine, 0.1% of Pluronic F68 (Sigma), 1 μg of pyrophosphatase (Roche), 500 nM Gemini-561, 1 μM coumarin acetate (Sigma) and 17.5 μg/mL T7 RNA polymerase (prepared in the laboratory). IVT mixture was loaded in a length of PTFE tubing and kept on ice during all experiment. PCR droplets were spaced and IVT droplets produced using a single stream HFE 7500 fluorinated oil (3M) supplemented with 2% (w/w) of fluorinated. Flow-rates (MFCS, Fluigent) were adjusted to generate 16 μL IVT droplets and maximize synchronization of 1 PCR droplet with 1 IVT droplet. Pairs of droplets were then fused with an AC field (400 V at 30 kHz) and the resulting emulsion was collected off-chip and incubated for 2 h at 37° C.

iii. Droplet analysis and sort. The emulsion was finally reinjected into an analysis and sorting microfluidic device at a frequency of about 150 droplets/s and spaced with a stream of surfactant-free HFE 7500 fluorinated oil (3M). The orange fluorescence (Gemini-561 in complex with the aptamer) of each droplet was analysed and the 1-2% most orange fluorescence droplets were sorted. The gated droplets were deflected into collecting channel by applying an AC fields (1000 V 30 kHz) and collected into a 1.5 mL tube. Sorted droplets were recovered from the collection tubing by flushing 200 μL of HFE fluorinated oil (3M). 100 μL of 1H, 1H, 2H, 2H-perfluoro-I-octanol (Sigma-Aldrich) and 200 μL of 200 μg/mL yeast total RNA solution (Ambion) were then added and the droplets broken by vortexing the mixture. DNA-containing aqueous phase was recovered, and the DNA recovered by PCR.

### RNA Probing

20 μg of RNA were first dephosphorylated for 20 min at 37° C. using 1 U FastAP (Fermentas) per μg of RNA. Upon phenol/chloroform extraction, and RNA precipitation, dephosphorylaoed RNA was 5′ labelled by incubating 5 μg of dephosphorylated RNA with 50 μCi of [P³²]γATP and 10 U of T4 polynucleotide kinase, with T4 PNK 1× buffer in a final volume of 15 μL during 1h00 at 37° C. prior to be phenol/chloroform extracted, precipitated and pelleted. Labelled RNA was then gel-purified and eluted from the gel by an overnight incubation at 4° C. and gently mixing in RNA Elution Buffer (500 mM of ammonium acetate and 1 mM of EDTA). Eluted radiolabelled RNA was extracted by phenol/chloroform treatment, precipitated in ethanol and pelleted as described above. The RNA is resuspended in nuclease-free water. The specific activity (cpm/μL) is calculated by measuring 1 μL in a radioactivity counter “Multi-Purpose Scintillator Counter” (Beckman) by Cerenkov counting. Labelled RNA (200,000 cpm) was renatured by heating it for 1 min at 90° C. then 1 min on ice and then pre-incubated at 20° C. for 15 min in a buffer containing 20 mM of this-HCl pH7.5, 1 mM of MgCl₂ and 150 mM of KCl. 1 μg of total RNA is then added to the preparation and RNAS were incubated with T1 enzyme (0.25 U, 0.5 U, I U), T2 enzyme (0.0125 U, 0.025 U, 0.05 U) and V1 enzyme (0.001 U, 0.002 U, 0.004 U) for 5 min at 20° C. (T1 and V1) or 10 min at 20° C. (T2) or water (Ctrl lame). The same amounts of digested products were loaded on a 10% denaturing 8 M urea acrylamide/bisacrylamide gel in parallel of an alkaline hydrolysis ladder and a denaturing T1 as described in Duval, M. et al. in RNA Structure and Folding Biophysical Techniques and Prediction Methods 29-50 (De Gruyter, 2013). The radiolabelled RNAS were then visualized on autoradiographic film.

### Cell Culture and Transfection

HeLa (ATCC® CCL-2™) and HEK293T (ATCC® CRL-3216™) cells were grown in Dulbecco's Modified Eagle Medium without phenol red (DMEM, Gibco-Invitrogen) supplemented with 10% fetal bovine serum (FES, Lonza), 1% L-Glutamine (Sigma Aldrich) and 1% antibiotic solution (Penicillin-Streptomycin, Sigma-Aldrich) at 37° C. in humidified atmosphere containing 5% CO₂. RNA-coding constructs were transfected directly into a 35 mm glass-bottomed imaging dish (IBiDi®) using FuGene HD (Promega) transfecting agent following recommended manufacturer protocol. Imaging experiments were performed between 16-24 h post-transfection.

### Cellular Imaging

Cells were seeded onto a 35 mm glass-bottomed imaging dish (IBiDi®) at a density of 3-5×10⁴ cells/well 48 h before the microscopy measurement. 16-24 h prior to imaging cells were transfected with corresponding DNA plasmid. For imaging, the culture medium was removed and the attached cells were washed with Opti-MEM (Gibco-Invitrogen). Next, the cells were incubated in Opti-MEM with Hoechst (5 μg/mL) to stain the nuclei and in the presence of Gemini-561 dye (0.2 μM) for 5 min, the living cells were washed twice with Opti-MEM and visualized in Opti-MEM or were fixed in 4% PFA in PBS for 5 minutes before being wash twice in PES. The images were acquired in epifluorescence mode with a Nikon Ti-E inverted microscope, equipped with CFI Plan Apo×60 oil (NA=1.4) objective, and a Hamamatsu Orca Flash 4 sCMOS camera. The acquisition settings were: Hoechst (ex. 395 nm, em. 510±42 nm), eGFP (ex: 470 nm, em: 531±40 nm), G561/o-Coral complex (ex: 550 nm, em: 595±40 nm) and Alexa-647 (ex: 638 nm, em: LP 647 nm). The images were recorded using NIS Elements and then processed with Icy software.

### Microinjection Experiments

Cells were seeded onto a 35 mm glass-bottomed imaging dish (IBiDi®) at a density of 1×10⁵ cells/well 24 h before the microscopy measurement. For imaging, the culture medium was removed and the attached cells were washed with Opti-MEM (Gibco-Invitrogen). Next, the cells were incubated in Opti-MEM with Hoechst (5 μg/mL) to stain the nuclei. In vitro transcribed and purified aptamers were preincubated with respective fluorogen for 10 min in selection buffer to form complex at corresponding concentrations indicated in figures. Microinjection parameters: Pi=90 [hPa]; Ti=0.3 [s]; Pc=10 [hPa]. The images were acquired in epi-fluorescence mode with a Nikon Ti-E inverted microscope, equipped with CFI Plan Apo×60 oil (NA=1.4) objective, and a Hamamatsu Orca Flash 4 sCMOS camera. The acquisition settings were: Hoechst (ex. 395 nm, em. 475±50 nm), Broccoli (ex: 470 nm, em: 531±40 nm); Corn (ex: 470 nm, em: 531±40 nm); Mango (ex: 470 nm, em: 531±40 nm); Coral (ex: 550 nm, em: 595±40 nm). The images were recorded using NIS Elements and then processed with Icy software.

### TA Cloning

The DNA contained in the libraries obtained after the last two rounds of droplet-based microfluidics screening were amplified by PCR as described above but using the DreamTaq® enzyme and buffer (Fermentas) instead of Phirell (Fermentas). PCR products were purified using of the “Wizard® SV Gel and PCR Clean-Up System” kit (Promega) and inserted into the cloning vector of the “insTAclone PCR Cloning” kit (Thermo-Scientific) following the manufacturer's recommendations by overnight ligation at 4° C. ElectroTEN Blue bacteria were then transformed by electroporation by the ligation mixture and plated onto a 2YT/agar/Ampicillin (100 μg/mL) plate.

Individual colonies were used to seed 20 μL of Phirell PCR mixture (see above) while the rest of the colony was introduced in 3 mL of liquid medium 2YT/Ampicillin (100 μg/ml) overnight at 37° C. under agitation. Upon thermocycling, 2 μL of PCR product were added to 18 μL of in vitro transcription mixture (see above) supplemented with 100 nM of Gemini-561. The mixture was then incubated at 37° C. in a qPCR machine (Stratagene MX300SP, Agilent Technologies) and the fluorescence of the reaction was monitored for 2 hours (ex/em 575/602 nm). Finally, plasmid DNA was extracted from bacteria of interest using “GenElute Plasmid Miniprep” kit (ThermoFisher) and sequenced by the Sanger method (GATC Biotech).

### Real-Time IVT Measurements

PCR products of each selection cycle were purified by the “Wizard® SV Gel and PCR Clean-up System” (Promega) kit and quantified by NanoDrop™. 50 ng of pure DNA was introduced into 38 μL of in vitro transcription mixture (see above) supplemented with 100 nM of Gemini-561. This mixture was then incubated at 37° C. in a real-time therm0cycler (Stratagene Mx3OOSP, Agilent Technologies) and the fluorescence was monitored as above.

### Fluorescence Measurement on Purified RNA

1 μM purified RNA was heated for 1 min at 90° C. and cooled at 4° C. for 1 min. The solution was then supplemented with 1 volume of a twice concentrated mixture containing 80 mM potassium phosphate buffer pH 7.5, 2 mM MgCl₂, 0.1% Tween 20, 200 mM of salt (KCl, NaCl, LiC or CsCl) and 100 nM Gemini-561. The mixture was then incubated for 10 min at 25° C. prior to measuring the fluorescence at 25° C. 011 a real-time thermocycler (ex/em 575/602 nm, MX 3005P, Agilent) or on microplate reader (ex/em 560/600 nm, SpectraMax iD3, Molecular Devices).

### Affinity Measurements

To measure Kd, the concentration of renatured and purified RNAS was progressively increased from 2.45 nM to 40 μM for SRB-2 and from 3.9 nM to 4 μM for 4C10 and o-Coral aptamer with 100 nM (for SRB-2) or 25 nM (for 4C10 and o-Coral) of Gemini-561 in 40 mM potassium phosphate buffer pH 7.5, 100 mM KCl, 1 mM MgCl₂ and 0.05% Tween 20. The fluorescence was measured on microplate reader (ex/em 580/620 nm, SpectraMax iD3, Molecular Devices).

### Expression Vectors Design

The sequences coding for o-Coral or 20 nucleotides from Broccoli aptamer (Ctrl) were introduced downstream a U6 promotor into a F30-scaffold contained into a pUC57 vector (Proteogenix) via a restriction (Sbfl)/ligation step. The entire sequences (pU6_o-Coral_F30 or pU6_CUI_F30) were then introduced into an eGFP-N1 vector (Clontech) using AflII restriction sites. Alternatively, o-Coral-F30 or Ctrl-F30 sequences were introduced directly in the 3′UTR of the eGFP coding sequence under the control of a CMV promotor by a restriction (Mfel)/ligation step.

### Results

### Design and Synthesis of Gemini-561 Fluorogen

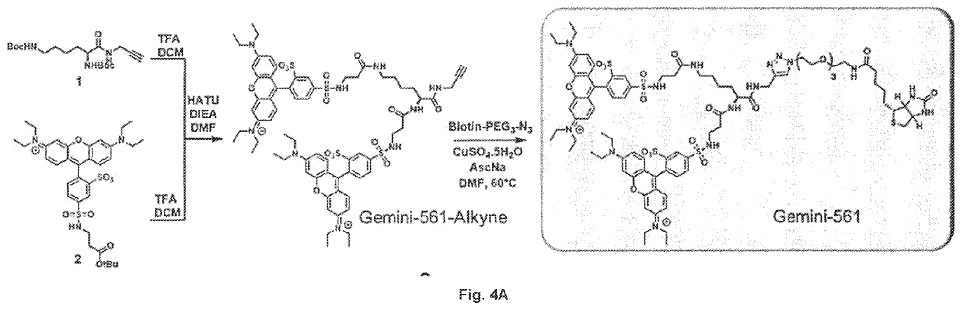

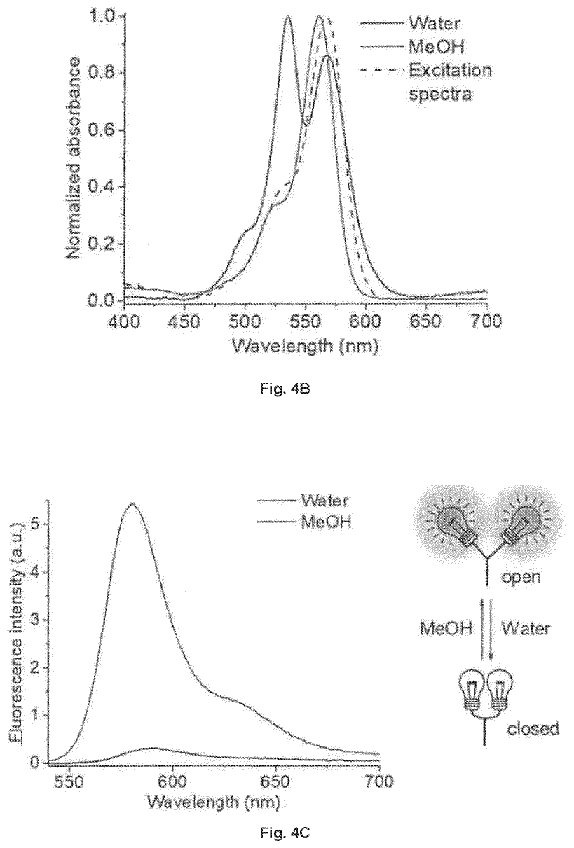

The inventors intended to develop an orange-red emitting fluorogen that would be efficiently excited with a common laser (530-560 nm), allowing multicolor imaging in combination with GFP-tagged proteins. Rhodamine fluorophores like SRB fulfil this requirement and also possess numerous advantages. First, due to their tendency to form H-aggregates and their ability to be quenched by different systems (e.g. spirolactonization, PET), rhodamines constitute efficient platforms to develop reliable fluorogenic sensors. SRB bears two sulfonate groups and upon functionalization becomes zwitterionic, i.e. non-charged. Compared to cationic rhodamines or non-charged fluorophores, this feature should increase the polarity of the molecule and thus enhance the water solubility and, at the same time, prevent from non-specific interactions in biological media. Finally, SRB features optimal photophysical properties including elevated quantum yield, good photostability and high molar absorption coefficient (about 100,000 M⁻¹·cm⁻¹). Gemini-561 was designed to promote the dimerization induced self-quenching of two SRBs. For this purpose, lysine, a natural amino acid, was chosen as a connector to provide a small distance between the dyes and thus ensure efficient π-stacking upon dimerization. Lysine (1) and SRB (2) derivatives were deprotected and coupled to lead to Gemini-561-alkyne (FIG. 4A). The latter was clicked to biotin-PEG-N₃ to yield Gemini-561.

### Spectroscopic Properties of Gemini-561

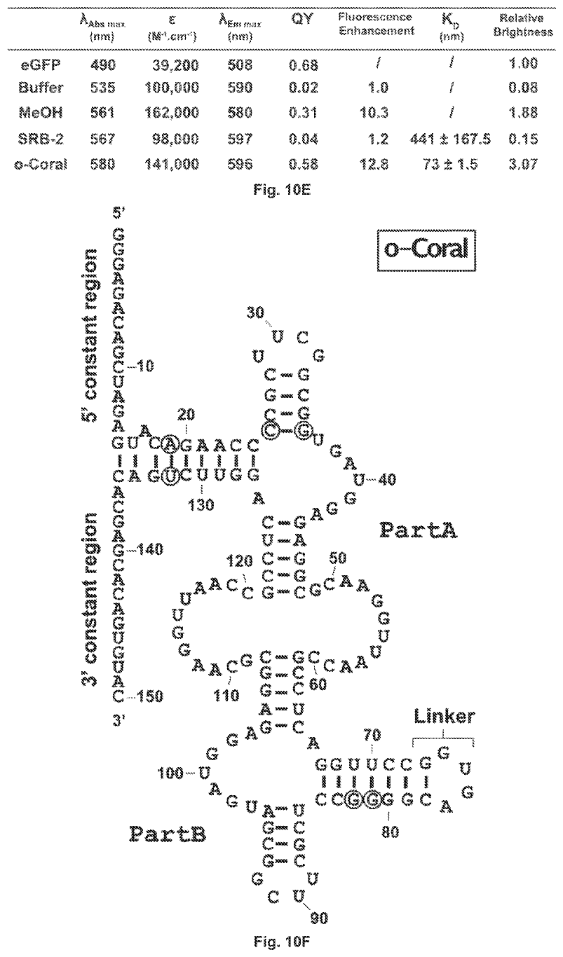

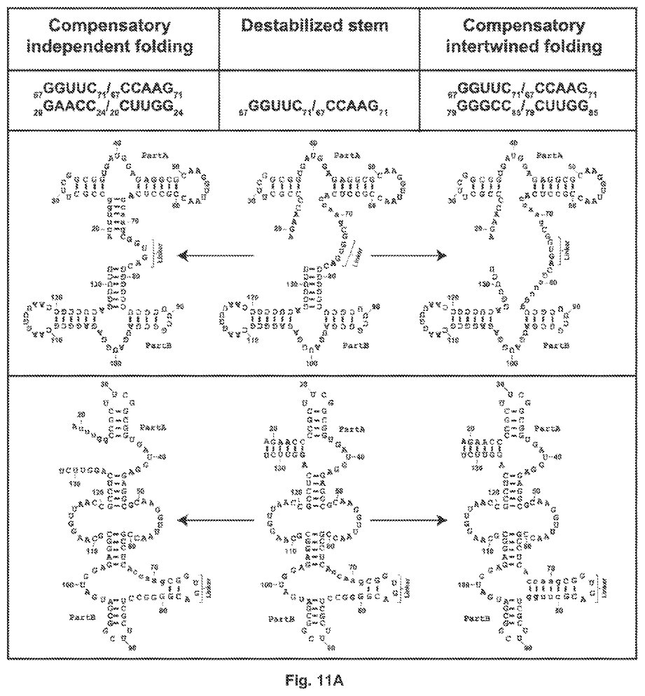

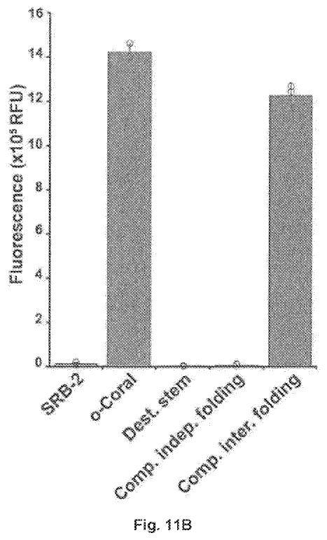

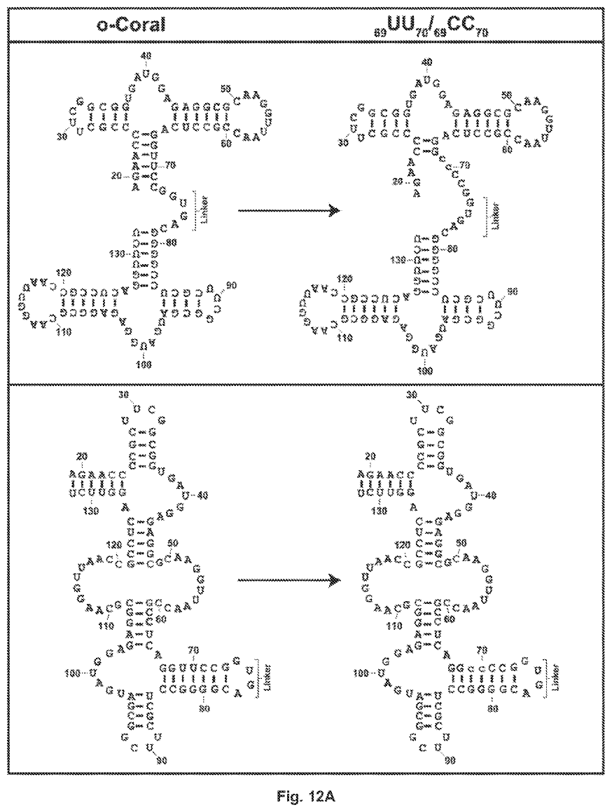

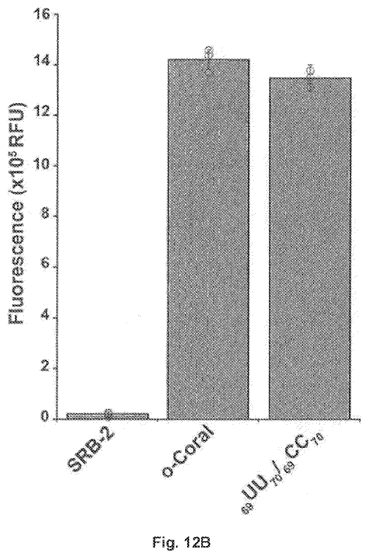

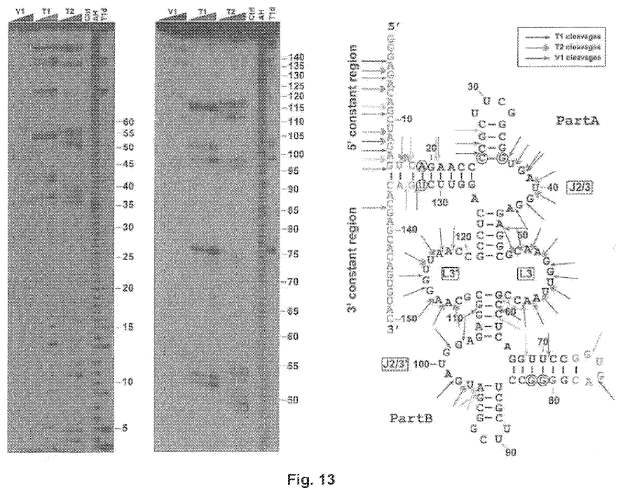

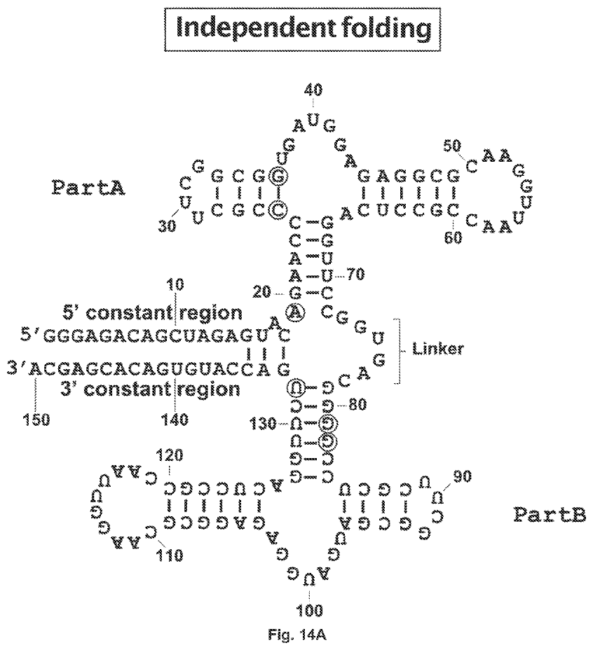

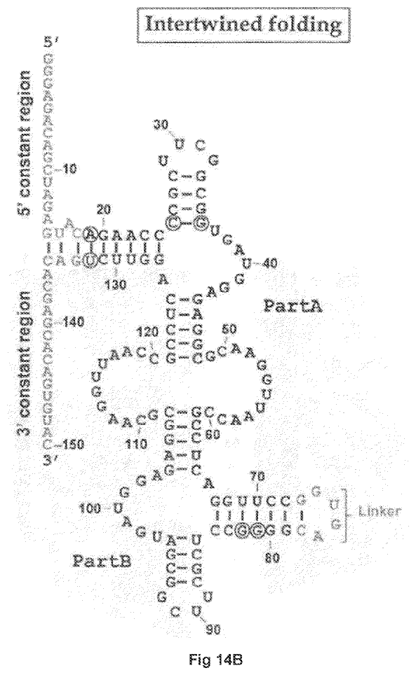

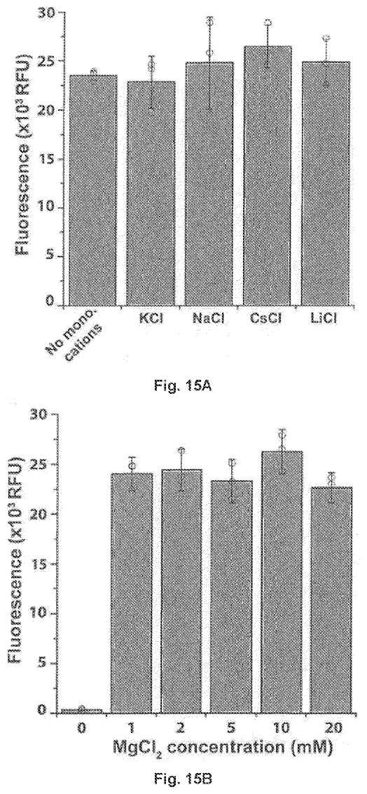

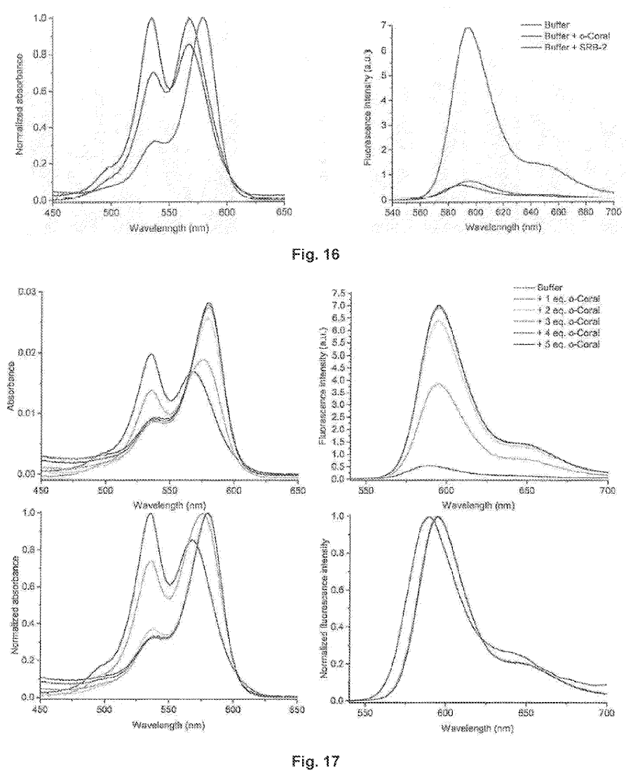

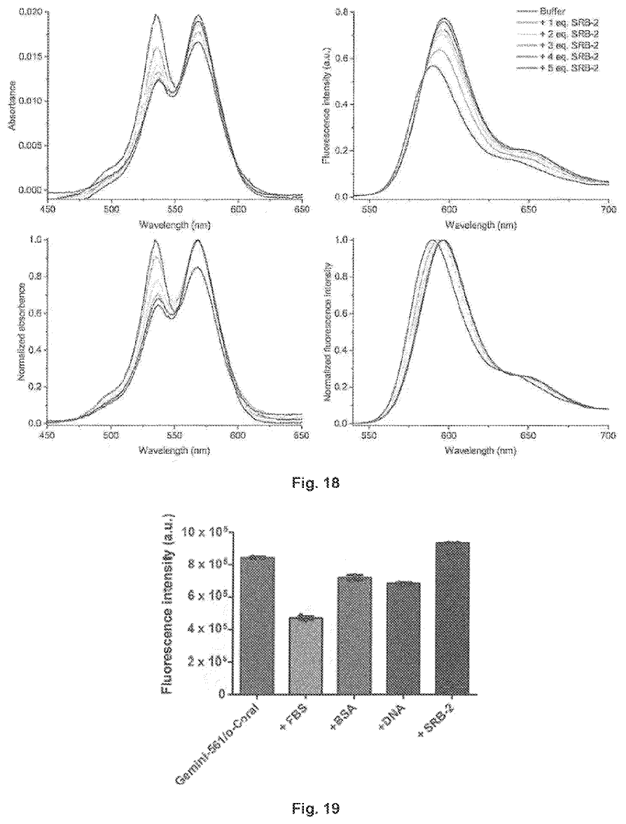

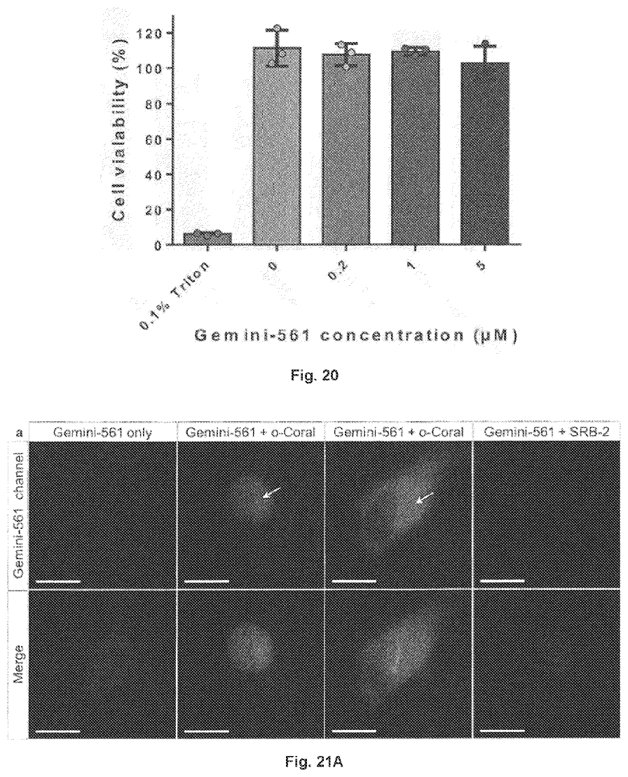

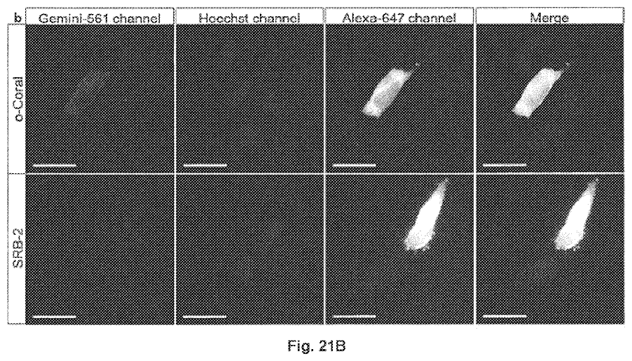

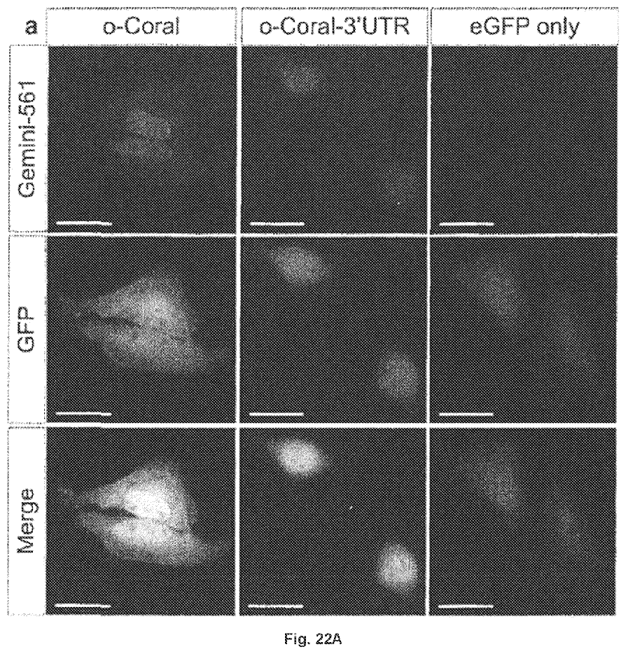

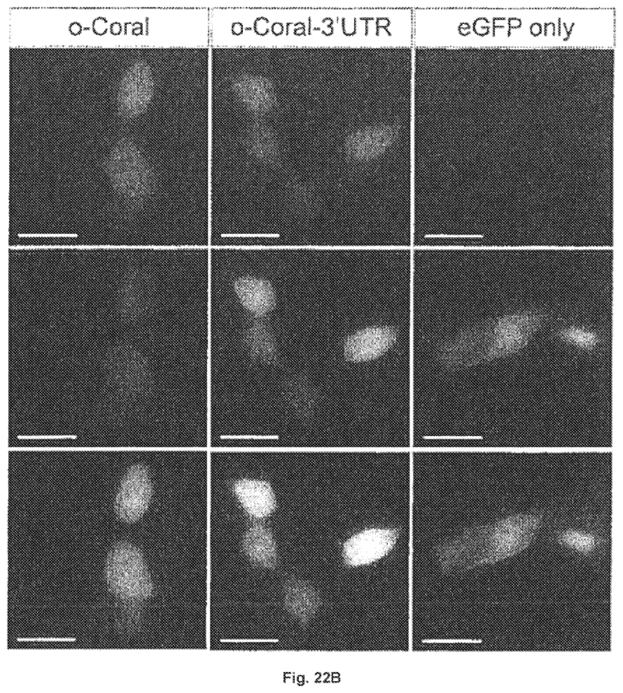

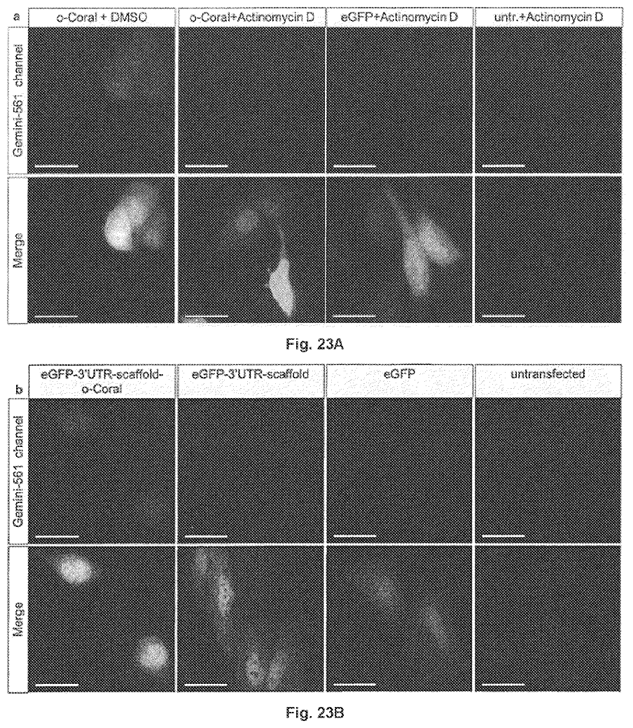

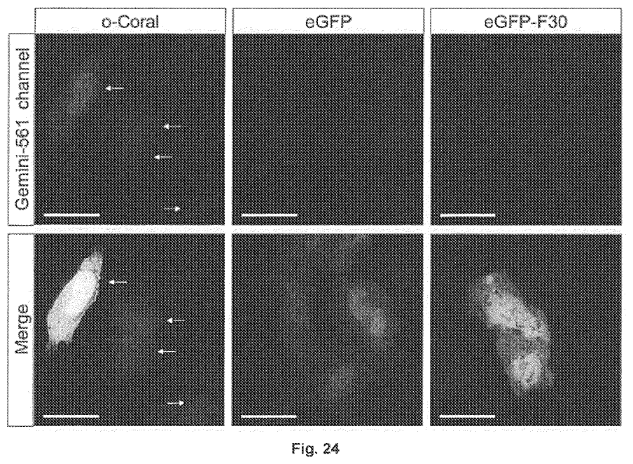

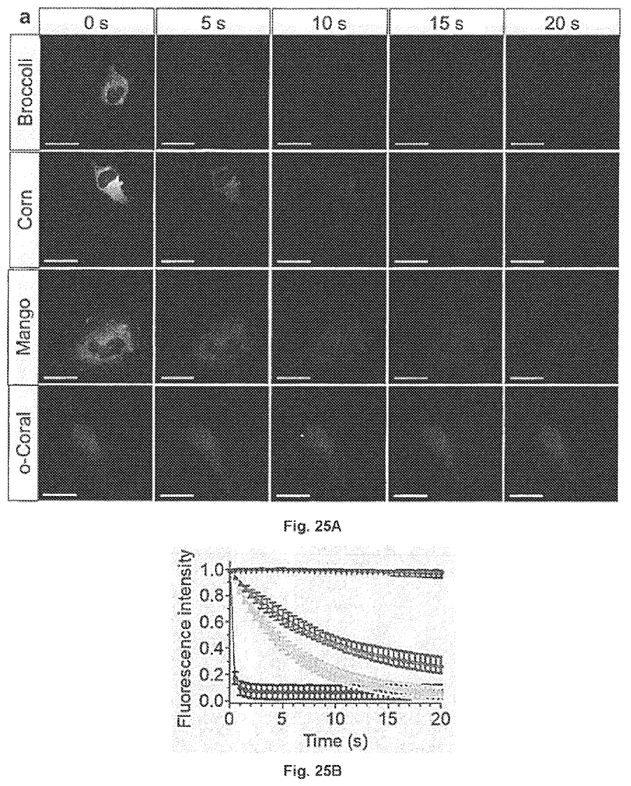

Gemini-561 fluorogenicity was first assessed by spectroscopic approach. In water, Gemini-561 displayed weak fluorescence intensity with a quantum yield value of 0.01. Moreover, a blue shifted band (530 nm) appeared in the absorption spectrum indicating the formation of dimeric H-aggregate (FIG. 4B), in line with earlier report on the squaraine dimers. Additionally, excitation spectrum showed that this band did not correspond to emissive specie (FIG. 4C), thus confirming a dimerization-induced quenching phenomenon. However, upon solubilization in methanol, the dimer opened and Gemini-561 displayed absorption and emission spectra similar to free SRB (FIGS. 4B and C) along with an impressive increase in the quantum yield value (0.31, FIG. 10E). In a second step, the non-specific opening of the dimer was evaluated in physiological media including PES, DMEM and Opti-MEM.

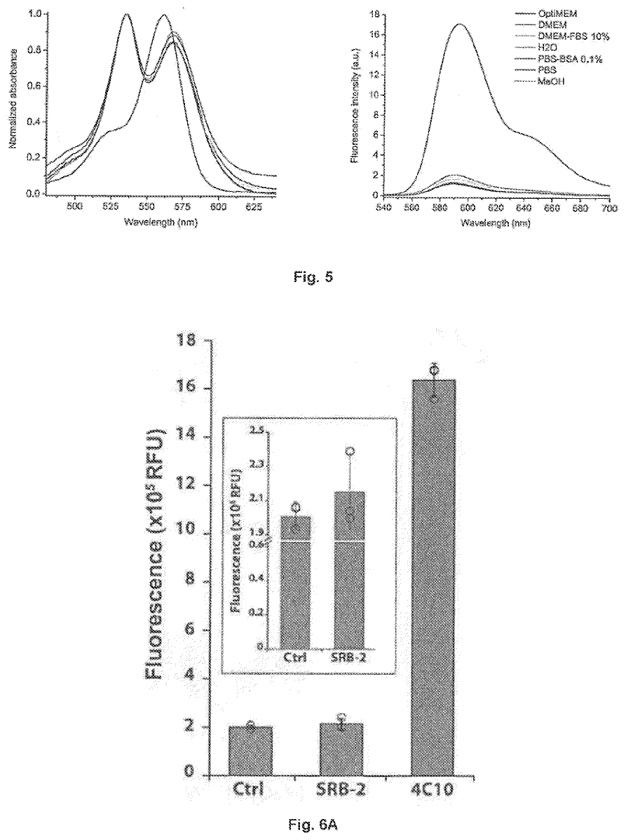

Gemini-561 proved to conserve its quenched form in various conditions including those in the presence of bovine serum albumin (BSA) or 10% fetal bovine serum (FES). These results suggest that Gemini-561 is not involved in non-specific interactions with proteins and lipoproteins that could provoke non-desired turn-on of the dimer (FIG. 5). Altogether, these experiments demonstrate that Gemini-561 constitutes an effective fluorogenic molecule compatible with biological media thus making it a promising candidate for selection of the RNA aptamer.

### Isolation of Gemini-561 Lighting-Up Aptamers by In Vitro Evolution

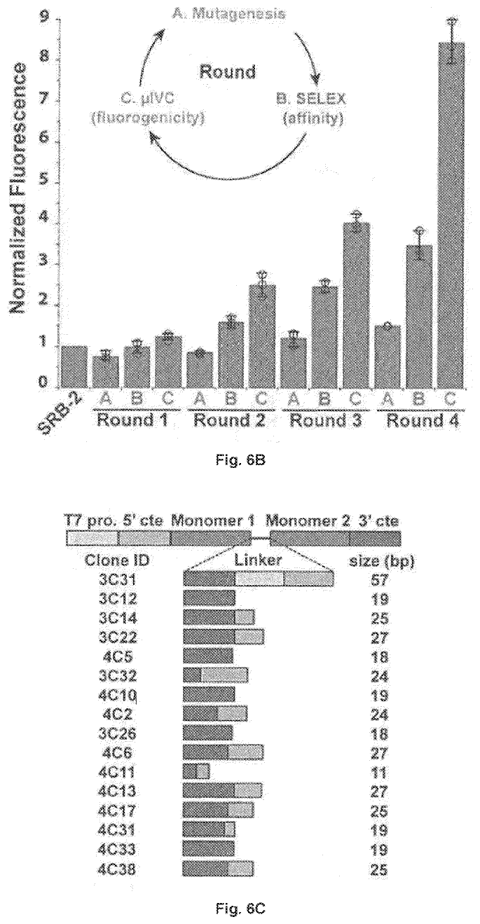

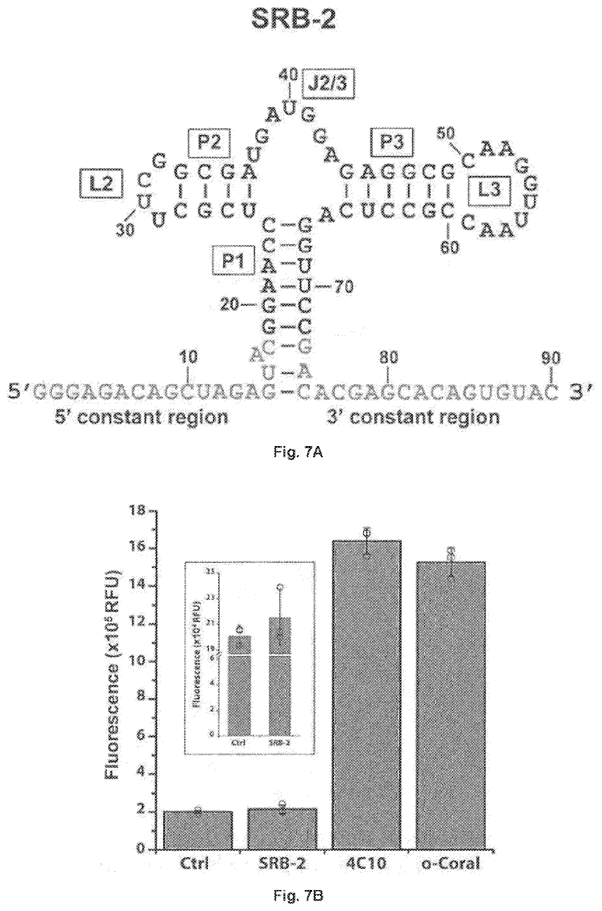

The inventors first studied SRB-2 aptamer, which was previously developed to specifically interact with sulforhodamine B and its derivatives. However, its capacity to turn on Gemini-561 fluorescence was poor (FIG. 6A, FIGS. 7A and B). This weak effect might be attributed to inhibition of dye-aptamer interaction by the dye-dye dimerization.

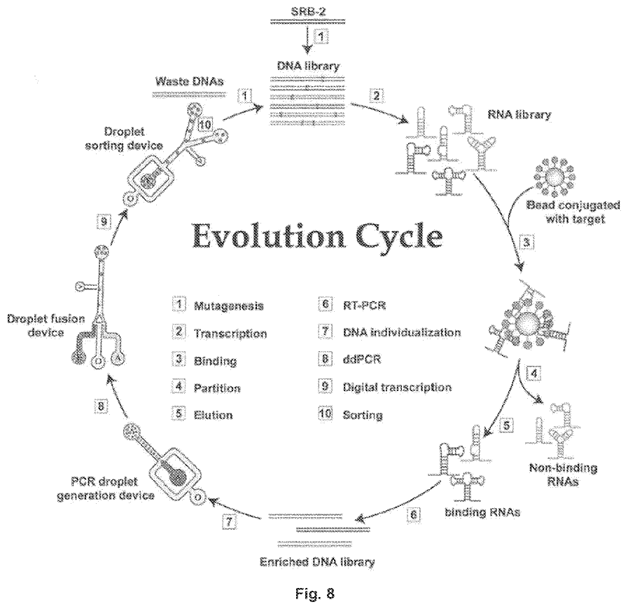

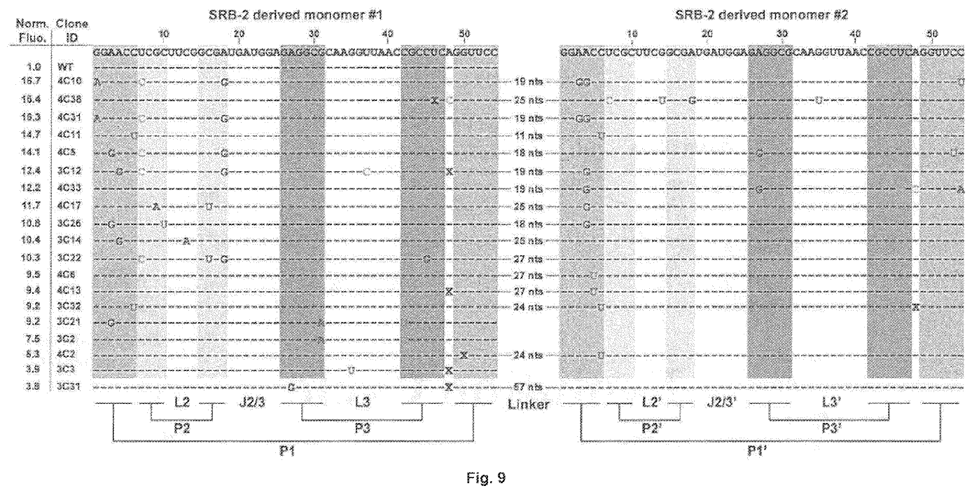

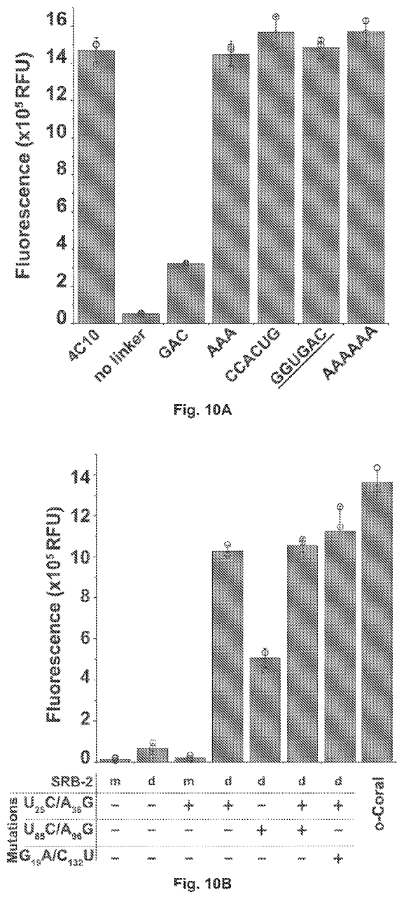

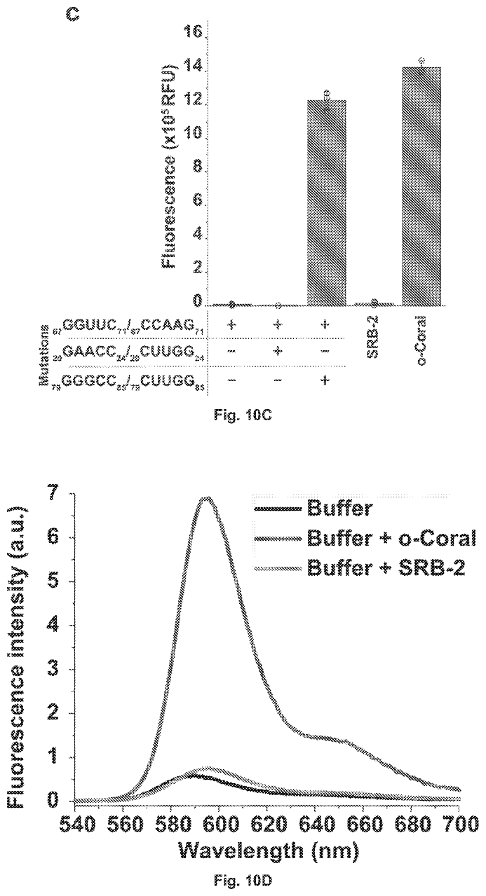

The inventors therefore started in vitro evolution of SRB-2 using a strategy combining SELEX in tandem with μIVC (FIG. 6B, FIG. 8) to isolate RNAs endowed respectively with both high affinity and high fluorogenic capacity. SRB-2 mutant library (about 3.4 mutations per gene) was first generated by error-prone PCR and subjected to a first round of SELEX during which RNAs were challenged to bind bead-immobilized Gemini-561. Upon stringent wash, bound aptamers were recovered, and reverse transcribed into cDNAs to which T7 RNA polymerase promoter was appended. Resulting genes were then subjected to a round of droplet-based microfluidic μIVC screening as previously described. Genes encoding fluorogenic RNAs were then recovered and used to prime a new round of error-prone PCR. A total of four such evolution cycles (mutagenesis, SELEX, μIVC) were performed and allowed to gradually improve the average fluorogenic capacity of the population (FIG. 6B). Cloning and analysing genes isolated from the two last rounds confirmed the overall success of the process since ˜40% of the tested sequences were significantly more fluorogenic than the parental SRB-2 aptamer. Surprisingly, 16 out of the 19 best mutants also displayed a size increase by about 2-fold resulting from complete duplication (dimerization) of SRB-2 sequence that occurred upon a recombination event at a variable position between the 5′ and 3′ constant regions of two aptamers (FIG. 6C and FIG. 9). The exact mechanism of this spontaneous recombination will require a dedicated study. In addition to this duplication, each optimized variant displayed 1 to 6 point mutations concentrated on PI and P2 regions of the SRB-2 (FIG. 9), while leaving region J2/3, P3 and L3 largely intact, in agreement with their proposed involvement in sulforhodamine B recognition.

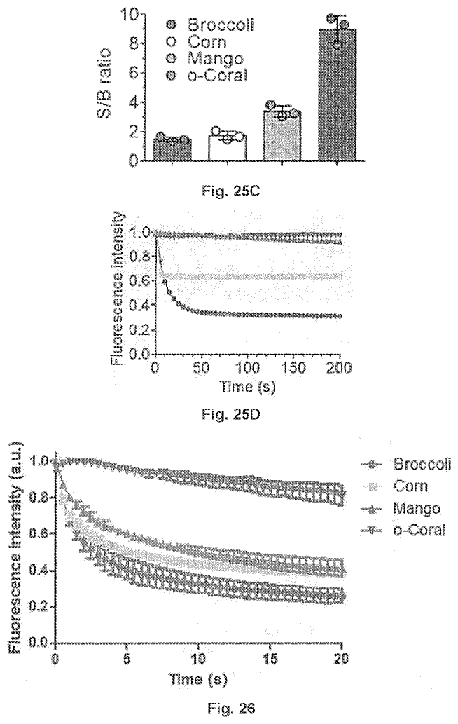

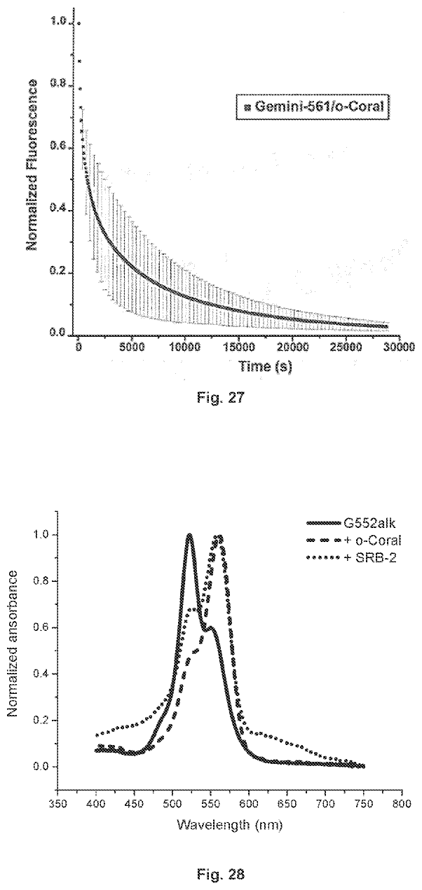

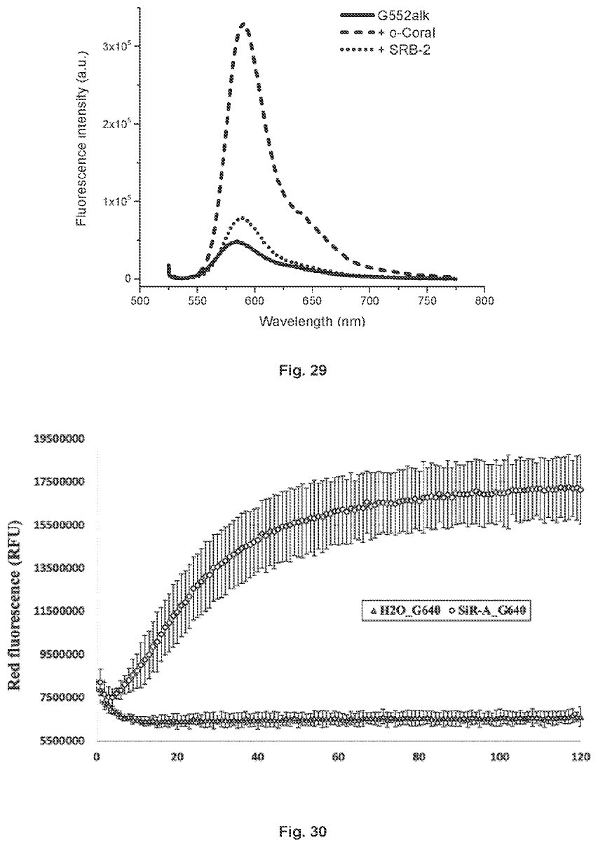

Among the different clones, 4C10, a duplicated variant displaying 6 point mutations, had a remarkably high activation capacity by forming with Gemini-561 a complex an order of magnitude more fluorescent than that of the parent SRB22 (FIG. 6A). 4C10 improvement correlates with a significant increase of affinity (about 73±1.5 nM and 441±167 nM for 4C10 and SRB-2 respectively, FIG. 10E). By further engineering 4C10, the inventors successfully reduced the 19-nucleotide long linker spacing the repeats down to 6 nucleotides while preserving intact fluorogenicity (FIG. 10A). Further reducing this length down to 3 nucleotides made aptamer fluorescence activation sensitive to the sequence of the linker. Therefore, the inventors pursued the study of a 6-nucleotide long linker 4C10 derivative, further named “o-Coral”, which conserved both fluorogenicity and affinity for Gemini-561 (FIG. 10E). The duplication event by itself accounts only partly for the high performances of o-Coral since a molecule made of two wild-type SRB-2 modules displays only 6% of o-Coral fluorescence (FIG. 10B). Progressive reimplantation of o-Coral mutations showed that all the mutations contributed to o-Coral function to a different degree with the double mutant U₂₅C/A₃₆G (a mutation found in a third of the 19 best mutants, FIGS. 9, 11A and B) having the predominant effect. Remarkably, simple introduction of U₂₅C/A₃₆G mutation into SRB-2 did not yield any improvement of the monomer, indicating a synergic effect of the mutation with the dimerization (FIG. 10B). Furthermore, U₂₅C/A₃₆G had a higher effect when present in the 5′ monomer, whereas introducing it in both monomers did not further improve the aptamer.

o-Coral Structural Characterization

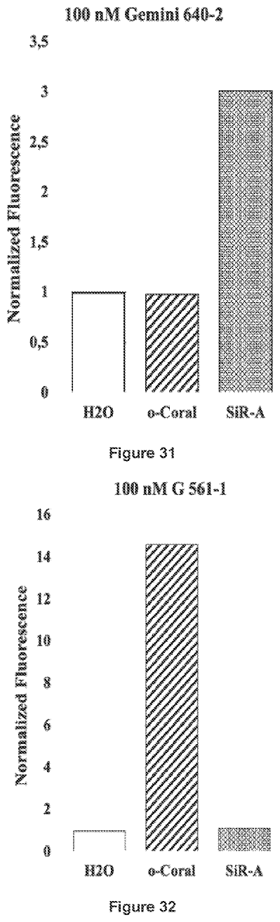

An important question raised in this work was to establish whether the dimerization of the RNA module was a simple duplication of the aptamer or if more important folding changes occurred. Enzymatic structural probing characterization (FIG. 13) was in agreement with an overall conservation of the structure initially proposed for the SRB-2 aptamer made of 3 stems and 3 unpaired regions. Yet, two RNA folding models could account for the probing signal observed: i) a model in which both SRB-2 derived modules fold independently (FIG. 14A) and ii) an intertwined folding (FIG. 14B). To discriminate both models, the inventors generated a mutant with a destabilized stem (₆₇GGUUC₇₁ changed for ₆₇CCAAG₇₁, FIGS. 11A end B) leading to the complete loss of fluorogenic capacity (FIG. 10E). The inventors then tested compensatory mutants relevant either to the independent folding model (₂₀GAACC₂₄ changed for ₂₀CUUGG₂₄) or to the intertwined structure (₇₉GGGCC₈₅ changed for ₇₉CUUGG₈₅). Interestingly, only the second compensatory mutant rescued o-Coral function (FIG. 10C), suggesting that o-Coral adopts an intertwined folding (FIG. 10F). This model received further support from a second mutant (₆₉UU₇₀ changed for ₆₉CC₇₀) expected to stabilize only the intertwined folding (FIG. 12). Finally, when looking carefully at the mutations selected during the evolution process, one can see that two of them (G₁₉A and C₁₃₂U) compensate each other in the intertwined model (FIG. 14B).

In the inventor's model, unpaired regions corresponding to J2/3 and L3 in the original SRB-2 aptamer and proposed to form the SRB-binding site are preserved (nucleotides 37-43, 49-60, 97-103 and 109-20). The three-dimensional structure of SRB-2 aptamer has not been established yet. However, even though pairs of G bases are found within and around these loops, the existence of a G-quartet structure common to many other light-up aptamers is unlikely since the complex was found to be insensitive to the nature of the monovalent cation added in the medium (FIG. 15A). Understanding how exactly the fluorogen and the aptamer interact together will require a dedicated structural characterization.

### Gemini-561/o-Coral Characterization in Solution

Detailed spectroscopic characterization of Gemini-56I/o-Coral complex revealed a significant red-shifted absorption and fluorescence emission (by 19 and 16 nm, respectively) compared to those in an “activating” solvent methanol (FIG. 10D and FIGS. 16 to 18), presumably because of interaction between the dyes and RNA nucleobases. Interestingly, the quantum yield of the complex also reached a higher value than Gemini-561 in MeOH (FIG. 10E), showing that the dye is well confined within o-Coral aptamer. Moreover, estimating the brightness of the module, as extinction coefficient×quantum yield, indicated that a single copy of o-Coral associate With Gemini-561 is more than 3 times brighter than eGFP (FIG. 10E), making it the brightest aptamer-based module described so far. Moreover, Fluorescence Correlation Spectroscopy (FCS) confirmed high brightness of Gemini-561/o-Coral complex (at least 1.14-fold as bright as single tetramethyl rhodamine, a close analogue of SRB) and suggested that this complex is composed of a single molecule of Gemini-561 bound to a single copy of o-Coral (Table 1).

TABLE 1
Fluorescence Correlation Spectroscopy
(FCS) analysis of the fluoromodules.

| Fluoromodule/ | | | | Size, | |
|---|---|---|---|---|---|
| reference dye | n | τ_{corr}, ms | C, nM | nm | Brightness |
| Tetramethylrhodamine | 11.64 | 0.032 | 50 | 1 | 1 |
| o-Coral/Gemini-651 | 43.22 | 0.22 | 185.71 | 6.86 | 1.14* |
| Fluorescein | 13.03 | 0.026 | 50 | 1 | 1 |
| Broccoli/DFHBI-IT | 14.16 | 0.16 | 54.35 | 6.27 | 0.18 |
| Corn/DFHO | 54.7 | 0.063 | 209.94 | 2.56 | 0.62 |

n—number of emissive species per excitation volume; τ_{corr}—correlation time; size—hydrodynamic diameter of fluorescent specie; brighmess with respect to one molecule of the standard (rhodamine B for o-Coral, fluorescein for Broccoli and Corn). Mango was excluded from this study since its photophysical properties did not fit the analysis settings. Concentrations of fluorogen and corresponding aptamer were systematically 200 nM and 1 μM, respectively.
*This value corresponds to 532 nm excitation, which is less efficient for excitation than that for the TMR standard, because of the red shifted absorption of the former.

The Inventors next tested the effect of magnesium ions on fluorescence of the complex. RNA very often uses magnesium ions as a co-factor to assist its folding and in vitro without any other stabilizing agent (i.e. polyamines) it may sometimes require concentration far exceeding the 1-3 mM available in the cell. Here, the inventors found that as few as 1 mM magnesium chloride was sufficient to obtain maximal fluorescence of Gemini-561/o-Coral (FIG. 15A). This value is in agreement with that recently reported for SRB-2 aptamer and shows that o-Coral is compatible with intracellular magnesium concentrations. The inventors then investigated the effect of physiological media on the Gemini-561/o-Coral complex and found that whereas 10% FBS slightly interferes with the aptamer-fluorogen interaction (FIG. 19), SRB2 aptamer, BSA or DNA did not challenge the fluorescence of the complex. Altogether, these data further support the compatibility of Gemini-561/o-Coral module in complex cellular environment.

### Imaging Gemini-561/o-Coral in Live Cells

Prior to applying Gemini-561/o-Coral module in cells, the inventors first checked for the potent cytotoxicity of the fluorogen. As expected, the cell survival was not affected by an extended incubation with Gemini-561 (FIG. 20). The inventors then validated the functionality of the fluorogenic module in live HeLa cells by micro-injecting the preformed Gemini-561/o-Coral complex either directly into their nucleus or into their cytoplasm of living HeLa cells (FIG. 21A). In both cases, an intense red fluorescence was readily observed in the presence of the module, whereas the injection of Gemini-561 alone and mixed with SRB-2 aptamer did not yield significant fluorescence (FIG. 21A), confirming that the Gemini-561 fluorescence is specifically induced by o-Coral and that this module is well suited for live cell applications. We also assessed the cell permeability of Gemini-561 by incubating HeLa cells with 200 nM fluorogen. After washing, RNA was microinjected along with Dextran-Alexa-647 conjugate. Both o-Coral and SRB-2 were successfully injected in the cells as attested by Alexa-647 fluorescence (FIG. 21B). However, Gemini-561 fluorescence inside the cytosol was observed only in the presence of o-Coral, suggesting both cell permeability of the fluorogen and its capacity to detect o-Coral inside live cells.

The inventors next evaluated the performances of their fluorogenic module with aptamers synthesized in situ by the cell machinery. To this end, o-Coral was inserted into a F30 scaffold and placed under the control of a U6 truncated promoter allowing the gene to be transcribed by RNA polymerase III (pol III), resulting in RNA homogeneously distributed in the cell. The plasmid also contained the eGFP-coding region placed under the control of an RNA polymerase II (pol II) promoter and used for identifying transfected cells by their green fluorescence. Taking advantage of Gemini-561 cell-permeability, the inventors repeated the experiment by expressing o-Coral gene in live Hela and HEK293T cells, and then incubated the cells with 200 nM Gemini-561 for 5 minutes prior to imaging. As expected, green fluorescent (GFP) transfected cells also displayed intense red (Gemini-561) fluorescence both in the nucleus and in the cytoplasm (FIG. 22, FIG. 23). This fluorescence correlated with o-Coral gene expression since inhibiting it with actinomycin D, a known inhibitor of RNA polymerases, led to the complete loss of the red fluorescence signal (FIG. 23A). Interestingly, during these live-cell imaging experiments, the inventors noticed cell-to-cell variations in the fluorescence intensity of the Gemini-561/o-Coral (FIG. 24), suggesting different transcription states as reported recently with a different aptamer. Taken together, these data show that, owing to elevated brightness and affinity of Gemini-561/o-Coral complex, a single copy of pol III-expressed o-Coral aptamer is sufficient for RNA detection in living mammalian cells using Gemini-561.

The inventors finally extended our study to Pol II transcripts by inserting o-Coral/F30 scaffold directly into the 3′ untranslated region of the gfp gene carried on the plasmid. Excitingly, they found that labelling gfp mRNA with a single copy of o-Coral was sufficient to detect an intense red fluorescence observed in GFP transfected cells (FIG. 22). The mRNA detection with Gemini-561/o-Coral module worked well in both HeLa and HEK293T cells, revealing fine difference in mRNA distribution. Indeed, in HEK cells the signal of mRNA was evenly distributed between nucleus and cytosol, similarly to GFP, whereas in HeLa cells the mRNA signal was stronger in the nucleus (FIG. 22). It is noteworthy to highlight that, to the best of inventors' knowledge, it is the first time that a mRNA labelled with a single copy of a light-up aptamer can be visualized without the need for extensive image processing.

### Comparison of Gemini-561/o-Coral to Other Aptamer Modules

To further demonstrate the advantage of their new fluorogenic module, the inventors systematically compared its performances with that other modules that use commercially available fluorogens, namely Broccoli, Mango III and the Corn. Moreover, to properly compare our data with those reported in the literature, aptamers were embedded into the RNA scaffold as they were previously characterized in (i.e. tRNA scaffold for Corn and F30 scaffold for Broccoli, Mango III and o-Coral). First, the inventors assessed photostability of the fluorogenic modules in solution with fixed concentrations of a fluorogenic dye (0.2 μM) and corresponding aptamer (1 μM) (FIG. 25D), where all the solution in a cuvette was irradiated with a laser light. To ensure that the four studied systems absorbed similar amount of photons during photobleaching, the applied irradiation power density (irradiance) inversely proportional to the extinction coefficient of the corresponding dye at the excitation wavelength used. DFHBI-IT/Broccoli complex showed very poor photostability as its intensity vanished within <1 s, in line with previous reports. Significantly higher photostability was observed for DFHO/Corn and TO1—biotin/Mango III, as their emission changed to much lower extent within 200 s of irradiation, which also corroborates with the earlier studies. Excitingly, emission intensity of Gemini-561/o-Coral did not show any change in the fluorescence intensity in these conditions, indicating that it is significantly more photostable than all three previously reported fluorogenic modules.

Second, the inventors compared brightness of the complexes at the single-molecule level using FCS (Mango system was not characterized because of incompatibility with our setup). Single-molecule brightness of DFHO/Corn and DFHBI-1T/Broccoli was correspondingly 0.62 and 0.18 (Table 1) with respect to that of reference dye fluorescein (at pH 9), which confirms much higher brightness of the DFHO/Corn module. On the other hand, the single molecule brightness of Gemini-561/o-Coral was 1.14 of that of reference dye tetramethyl rhodamine in water (Table 1). Taking into account that fluorescein and TMR exhibit similar brightness in the inventors' FCS setup, the new fluorogenic module, Gemini-5611/o-Coral, is about 2-fold brighter than DFHO/Corn in the single molecule experiments.

On the next step the inventors performed cellular experiments where the fluorogen/aptamer complex was preformed and then microinjected into live cells. Right after microinjection, images were taken (FIG. 25A) and the signal/background ratio (S/B) was measured. Gemini-561/o-Coral system provided images with a S/B value of 9±0.9, which was significantly higher than those obtained with other three studied fluorogenic modules (FIG. 25A). Images taken after different times of irradiation showed that fluorescence intensity within the cells decayed much slower for Gemini-561/o-Coral compared to other studied fluorogenic modules (FIGS. 25A and B), which confirmed superior photostability of the former. In an alternative experiment, cells were incubated in the presence of the fluorogenic molecules and then the corresponding aptamer was microinjected. In this case, the obtained photobleaching curves also showed that Gemini-561/o-Coral was significantly more photostable than other three reference aptamer-based systems (FIG. 26), allowing >20 s continuous imaging with only a minor loss of fluorescence intensity. Remarkably, Gemini-561/o-Coral fluorescence can still be detected upon several hours of constant illumination (FIG. 27).

### Discussion

Light-up aptamers gained their niche as powerful genetically encoded RNA imaging tools. Due to its high modularity and available SELEX methodologies, a palette of the fluorogen-aptamer systems was discovered to shine light on complex cell machinery. Unfortunately, limited brightness and photostability of aptamer-based fluorogenic modules developed to date narrow their broad application. In this work the inventors developed and characterized Gemini-561/o-Coral, a new RNA-based fluorogenic module displaying high brightness, affinity and photostability making it, to the best of inventors' knowledge, one of the brightest module described so far in the literature. the inventors reached this goal mainly through the combined use of two innovations. First, the fluorogen Gemini-561 which consists of two copies of the bright and photostable sulforhodamine B dye that self-aggregates into a poorly emissive specie able to rapidly enter the live cells. This quenching mode is interesting since, upon activation, both fluorophores become strongly fluorescent making such a probe brighter than any monomeric probe described to date. Second, the inventors developed a light-up RNA aptamer using a powerful integrated in vitro evolution strategy combining rounds of mutagenesis and SELEX in tandem with μIVC screening. In this scheme, the SELEX step allows isolating RNAs with highest affinity for the Gemini-561 probe, whereas the μIVC isolates the most fluorogenic sequences. The inventors finally obtained o-Coral that efficiently opens the aggregated dimer Gemini-561 through binding to each sulforhodamine B moiety. Using the same overall strategy, it should be possible to further expand RNA imaging toolbox by developing new orthogonal modules made of dimeric (Gemini) fluorophores of any desired colour while selecting new Coral aptamers that can specifically activate them.

The intertwined dimerized structure of o-Coral may also be advantageous for the future engineering of aptamers. Indeed, both L2 and L2′ loops together with the linker region are highly amenable to sequence modification and are attractive sites for inserting other sequences (e.g. sensing aptamers). By doing so, o-Coral could be converted into complex multi-inputs logic gates or biosensors.

o-Coral is readily expressed in mammalian cells where it forms a bright complex with Gemini-561 that is otherwise not activated by cell components. Interestingly, o-Coral does not seem to possess the G-quadruplex organization shared by most of the other structurally characterized light-up aptamers. This is of particular interest when considering a recent report suggesting that most of the RNA G-quadruplex domains would be kept globally unfolded in mammalian cells, suggesting that G-quadruplex-based RNA may not be optimal for being used in living cells. Direct comparison of Gemini-561/o-Coral with the most representative aptamer-based fluorogenic modules in live cells showed clear advantages of the new module in terms of brightness and photostability. Moreover, their estimated 3-fold higher brightness than GFP suggests that they can be brighter than a single copy of MS2-GFP module. Superior characteristics of Gemini-561/o-Coral module enabled imaging of mRNA by integrating just a single copy of the aptamer (o-Coral), which has remained a challenge so far in this technology. Overall, Gemini-561/o-Coral system significantly strengthens the toolbox for RNA imaging and shows a new direction in the development of ultrabright fluorogenic aptamer-based modules.

### Example 2: Synthesis of Gemini 552-Alkyne and its Response to o-Coral

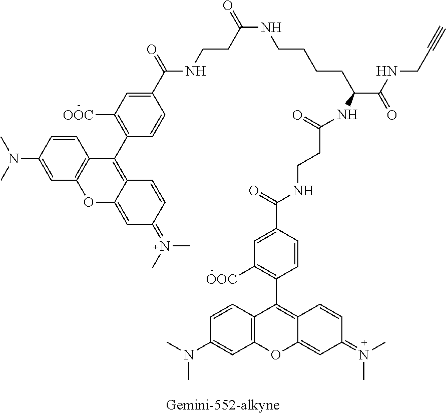

Method of synthesis of Gemini-552-Alkyne.

5-Carboxy-tetramethylrhodamine (20 mg, 47 μmol, 1 eq) was combined with DSC (18 mg, 70 μmol, 1.5 eq) in DCM (2.5 mL). After adding Et₃N (39 μL, 280 μmol, 6 eq) and DMAP (29 mg, 232 μmol, 5 eq), the reaction was stirred at room temperature for 1.5 h and protected from light. After that, deprotected diamine ((S)—N,N′-(6-oxo-6-(prop-2-yn-1-ylamino)hexane-1,5-diyl)bis(3-aminopropanamide), 7.6 mg, 23 μmol, 0.5 eq) was added. The reaction was stirred 16 h at room temperature, then solvent was removed by concentrating in vacuo. The crude material was purified by reverse phase HPLC to provide 16 mg (30%) of as a dark violet solid. HRMS (ESI+), calc. for C₆₉H₆₇N₉O₁₁ [M+2H]⁺ 1149.51. found 1149.51. ¹H NMR (400 MHz, Methanol-d₄) δ 8.65-8.57 (m, 2H), 8.06 (ddd, J=10.1, 7.9, 1.8 Hz, 2H), 7.34 (dd, J=7.9, 1.3 Hz, 2H), 7.27-7.20 (m, 4H), 7.01 (dtd, J=9.5, 2.4, 1.2 Hz, 3H), 6.92 (dd, J=4.0, 2.4 Hz, 4H), 4.39 (dd, J=9.3, 4.8 Hz, 1H), 3.99 (t, J=2.6 Hz, 2H), 3.75 (ddt, J=13.7, 9.5, 6.7 Hz, 4H), 3.37 (p, J=1.6 Hz, 6H), 3.30 (d, J=1.8 Hz, 31H), 3.23 (t, J=6.6 Hz, 2H), 2.78-2.66 (m, 2H), 2.67-2.55 (m, 3H), 1.97 (t, J=7.4 Hz, OH), 1.92-1.30 (m, 3H), 1.22 (d, J=6.2 Hz, 1H). ¹³C NMR (101 MHz, Methanol-d4) δ 172.56, 167.55, 157.51, 157.49, 157.20, 157.17, 135.58, 131.15, 131.15, 129.44, 128.15, 125.25, 113.28, 95.96, 95.96, 79.27, 70.83, 39.46, 36.58, 36.58, 35.30, 31.22, 28.63, 28.11, 23.85, 22.71.

### Results:

The spectroscopic response of Gemini-552 to o-Coral aptamer was studied in an aqueous buffer by absorption and fluorescence spectroscopy. It can be seen that absorption spectrum of Gemini-552 in a buffer presents a short-wavelength band, typical for the self-quenched H-aggregate (FIG. 28). In the presence of o-Coral, this band decreases with formation of a long-wavelength band, suggesting that Gemini-552 recognizes the aptamer, which leads to opening of the dimer. In the fluorescence spectra the addition of o-Coral leads to significant increase in the fluorescence intensity (FIG. 29), confirming the capacity of Gemini-552 to detect the aptamer through a fluorescence turn on. In case of a wild type (monomeric) aptamer (SRB-2), the spectroscopic effect and the fluorescence enhancement was much less pronounced, indicating that the interaction of Gemini-552 and o-Coral is specific. This example shows that the concept of the dye dimers can be applied to carboxy-derivatives of rhodamine dyes (like tetramethylrhodamine).

### Example 3 Aptamer-Mediated Activation of Gemini 640-2

This example shows that Gemini 640-2 (molecule 8) can be specifically activated by an RNA aptamer evolved to recognize the silicon-rhodamine moiety. Here, we used the SIR-A aptamer described in DOI/10.1021/jacs.9b02697.

### Method:

A template DNA
(SEQ ID NO: 34)
(5′-GGGAGACAGCTAGAGTACGGCCACCGGGTTTGAAAACCTGGCTG
CTTCGGCAGTTGTATCCTTTGGCCGACACGAGCACAGTGTAC-3′),

in which the SIR-A coding region (underlined sequence) was surrounded by two extensions (italicized sequence), was first PCR-amplified by introducing 1 ng of template DNA (IDT) into 100 μL of reaction mixture containing 50 pmoles of Fwd primer (5′-CTTTAATACGACTCACTATAGGGAGACAGCTAGAGTAC-3′ (SEQ ID NO: 35) adding T7 RNA polymerase promoter [bolded sequence] upstream the template), 50 pmoles of Rev primer (5′-GTACACTGTGCTCGTGTC-3′ (SEQ ID NO: 36)), 0.2 mM of each dNTPs, 1 U of Q5 DNA polymerase (New England Biolabs) and the corresponding buffer at the recommended concentration. The mixture was then thermocycled starting with an initial step of denaturation of 30 sec at 95° C. followed by 25 cycles of: 5 sec at 95° C. and 30 sec at 60° C. The PCR products were purified using “Monarch PCR purification kit” (New England Biolabs) following supplier recommendations and the recovered DNA quantified determined by NanoDrop™ measurement.
20 ng of purified PCR product were then introduced into 40 μL of in vitro transcription mixture containing 2 mM each NTP (Larova), 25 mM MgCl₂, 44 mM Tris-HCl pH 8.0 (at 25° C.), 5 mM DTT, 1 mM Spermidine, 1 μg of pyrophosphatase (Roche), 500 nM Gemini 640-2 (molecule 8), and 17.5 μg/mL T7 RNA polymerase (prepared in the laboratory). This mixture was then incubated at 37° C. into a microplate reader (SpectraMax iD3, Molecular Devices) and the red fluorescence (ex./em. 640 nm/680 nm) monitored every minute.

### Results:

Red fluorescence constantly increased in tubes containing the SIR-A template (i.e., gray circles on FIG. 30), whereas no signal was observed in the absence of template (i.e., gray triangles on FIG. 30). These data demonstrate that the fluorescence of Gemini 640-2 (molecule 8) can be activated ˜2.5-fold by an aptamer recognizing the Silicon-Rhodamine moiety. Moreover, incubating Gemini 640-2 (molecule 8) with an unrelated aptamer (i.e., o-Coral, the specific activator of Gemini 561-2 (molecule 7)) does not yield any fluorescence (FIG. 31), demonstrating the lack of non-specific activation of Gemini 640-2 (molecule 8). Furthermore, whereas Gemini 561-2 (molecule 7) is activated by o-Coral aptamer as expected (FIG. 32), it stays insensitive to SIR-A, further reinforcing the great specificity of both systems.

1. A molecular complex emitting fluorescent light comprising, or consisting essentially of:
a fluorophore, and
a nucleic acid molecule,
wherein said fluorophore has of the following formula 1 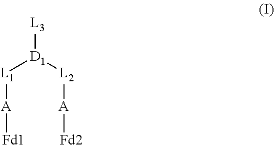
wherein
independently from each other, Fd1 and Fd2 are fluorescent dyes,
D₁ represents a group chosen from: 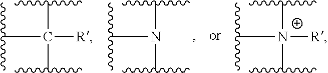
or from a cyclo(C₃-C₇)alkyl, a monocyclic aromatic group, heterocyclic group or a monocyclic non-aromatic, alkane or heterocyclic group, wherein R′ represents a hydrogen atom or a (C₁-C₈)alkyl, linear or cyclic, saturated or not,
independently from each other, L1 and L2 are covalently bounded to D₁, and are chosen from the group consisting of a single bond; a linear or branched alkyl group having from 1 to 24 carbon atoms, at least one of said carbon atoms being replaced by an heteroatom, or not, said alkyl group being substituted or not by an amido, an amino, a keto, an oxy, a carboxyl group, a linear or branched unsaturated or not alkyl group having from 2 to 24 carbon atoms, at least one of said carbon atoms being replaced by an heteroatom, or not, said alkyl group being substituted or not by an amido, an amino, a keto, an oxy, a carboxyl group;
L₃ is a hydrogen atom or a linear or branched alkyl group having from 1 to 24 carbon atoms (C₁-C₂₄), at least one of said carbon atoms being replaced by an heteroatom, e.g. O, N, S, or not, said alkyl group being substituted or not by an amido, an amino, a keto, an oxy or a carboxyl group or a linear or branched unsaturated or not alkyl group having from 2 to 24 carbon atoms, at least one of said carbon atoms being replaced by an heteroatom e.g. O, N, S, or not, said alkyl group being substituted or not by an amido, an amino, a keto, an oxy, a carboxyl group possibly substituted by a functionalizable moiety or a functional molecule; and
A is a C₁-C₁₂ alkyl, linear or cyclic, possibly substituted or an aryl, preferably a phenyl, substituted or not,
said fluorophore being submitted to quenching or energy transfer when it is not associated to said nucleic acid molecule in aqueous solution,
wherein said nucleic acid molecule is able to activate fluorescence of said fluorophore in an aqueous solution, when interacting with said fluorophore, and
wherein said nucleic acid molecule is able to specifically interact, in a sequence specific manner, with said fluorophore.

2. The molecular complex according to claim 1, wherein Fd1 and Fd2 are represented by formula 2 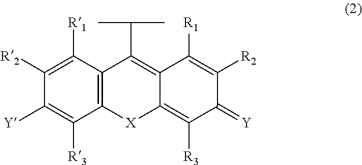
Wherein
X is NH, C(R)₂, O, Si(R)₂Ge(R)₂Sn(R)₂P(R)₂B(R)₂S, SO₂, Se, Te, TeO, where R can be alkyl or aromatic groups, or O, O-alkyl, sulfonyl such as sulfonate (SO3-) or sulfonamide;
Y is O, N—R₆ or 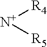
Y′ is O—R′₆ or N—R′₇ or 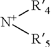
R₁ and R′₁ independently from each other, are H, a halogen atoms or a (C₁-C₁₈) alkyls, linear or cyclic, possibly branched,
R₂, R′₂, R₃, R′₃ can be H, sulfonyl such as sulfonate (SO3-) or sulfonamide;
R₂ and R₄ may form, together with the atoms of the carbon cycle to which R₂ is connected to, at least one fused aromatic heterocycle, said heterocycle cycle having 5 to 9 atoms,
R′₂ and R′₄ may form, together with the atoms of the carbon cycle to which R′₂ is connected to, at least one fused aromatic heterocycle, said heterocycle cycle having 5 to 9 atoms,
R₅ and R₃ may also form, together with the atoms of the carbon cycle to which R₃ is connected to, at least one fused aromatic heterocycle, said heterocycle cycle having 5 to 9 atoms,
R′₅ and R′₃ may also form, together with the atoms of the carbon cycle to which R′₃ is connected to, at least one fused aromatic heterocycle, said heterocycle cycle having 5 to 9 atoms,
R₄ and R₅ may also form at least one fused aromatic heterocycle, said heterocycle cycle having 3 to 9 atoms,
R′₄ and R′₅ may also form at least one fused aromatic heterocycle, said heterocycle cycle having 3 to 9 atoms, and
R₄, R′₄, R₅, R′₅, R₆ and R′₇, independently from each other, are polymethylene unit having 1 carbon to about 20 carbons, inclusive, optionally comprising at least one hetero atom selected from N, O and S.

3. The molecular complex according to claim 1, wherein said fluorophore has the following formula 3: 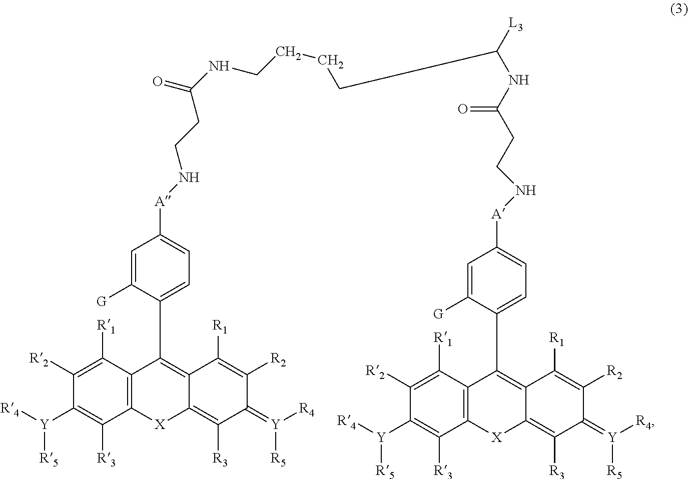
Wherein R₁, R′₁, R₂, R′₂, R₃, R′₃, R₄, R′₄, R₅, R′₅, R₆ and R′₇ and L₃ are as defined above, and A′ and A″ are independently from each other ether bond, ester, thioether, thioester, amide, sulfonamide, carbamate, thiocarbamate urea or thiourea,
Wherein G is H, an alkane (CH3), amido, an amino, a keto, an oxy, a carboxyl, a sulfo, sulfonyl or sulfonate group), a halide atom.
G can be in ortho, or meta or para position and can be repeated on the benzyl cycle.
A′ and A″ can be in ortho, meta or para position.

4. The molecular complex according to claim 1, wherein said -A-Fd1 and -A-Fd2 are one of the following fluorophores: Rhodamines, non-N-Alkylated Rhodamine, Ethyl-alkylated rhodamine or Silicon-Rhodamine.

5. The molecular complex according to claim 1, wherein said fluorophore is one of the following compounds having the following formulas:

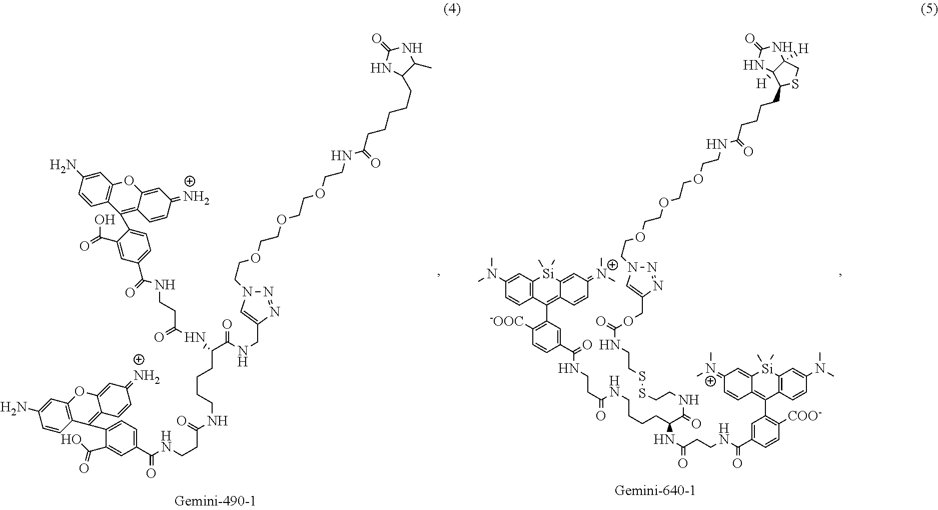

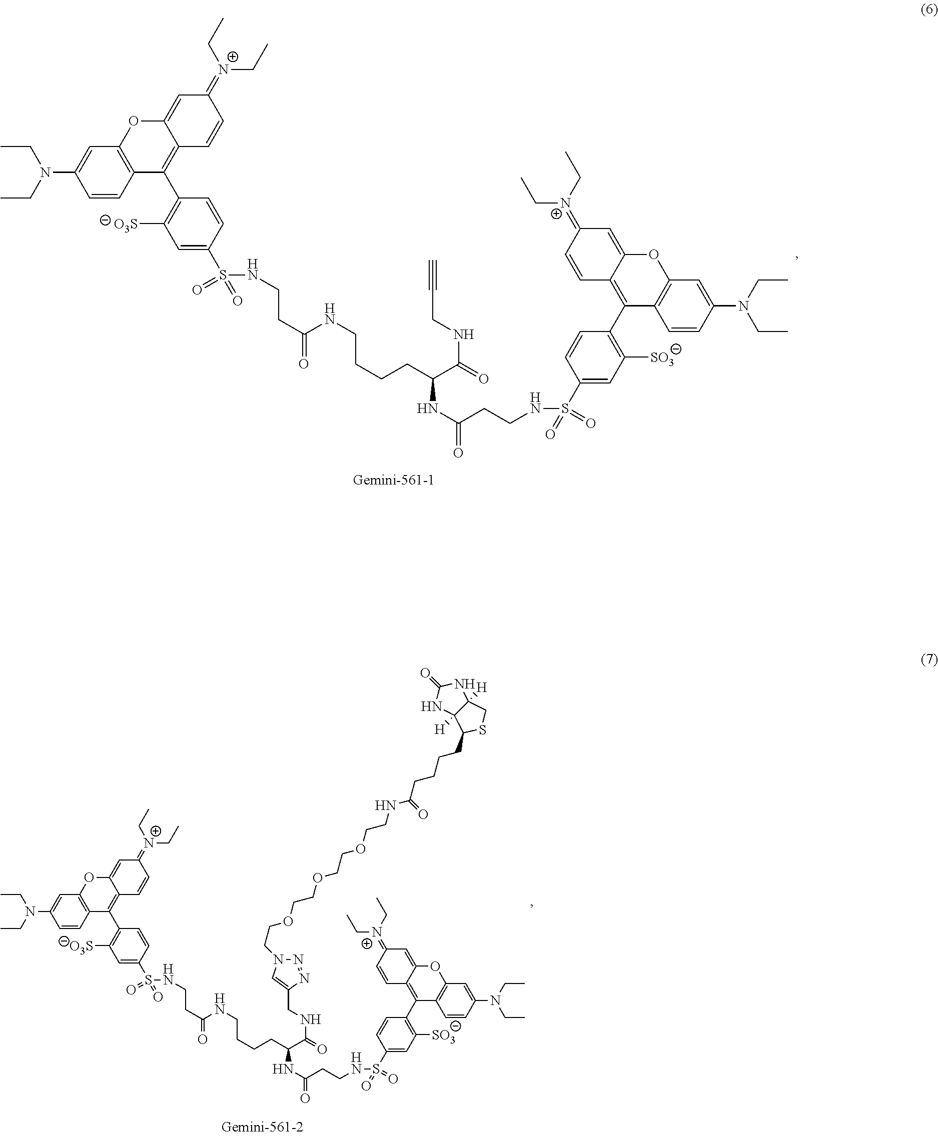

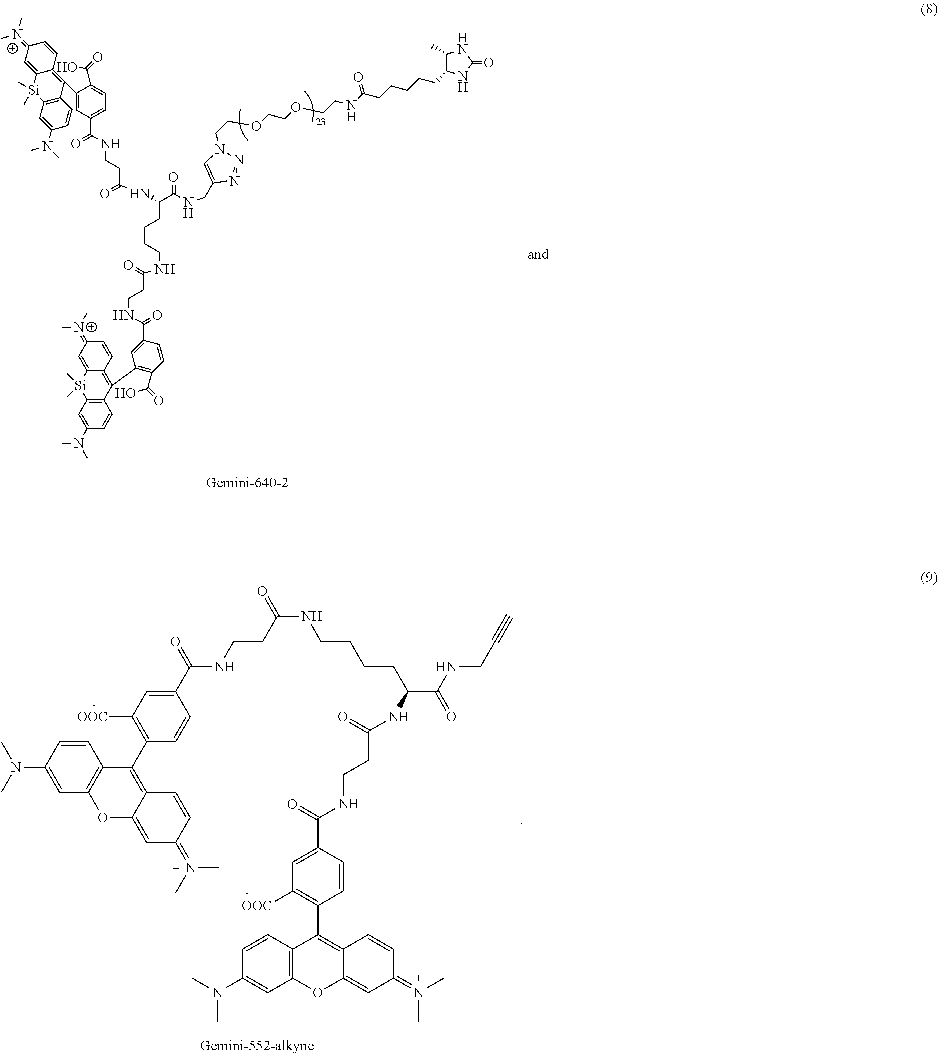

6. The molecular complex according to claim 1, wherein said complex harbors a brightness at least 3 fold higher than the brightness of free uncomplexed fluorophore and wherein said nucleic acid molecule has a KD affinity of at most 500 nM for said fluorophore.

7. The molecular complex according to claim 1, wherein said nucleic acid molecule comprises a first and a second region, said first and second regions being such that:
the first region comprises the nucleotide sequence of SEQ ID NO: 1; and
the second region comprises the nucleotide sequence of SEQ ID NO: 2, provided that said nucleic acid molecule is not the nucleic acid molecule as set forth in SEQ ID NO: 3.

8. The molecular complex according to claim 1, wherein the nucleic acid molecule comprises one of the nucleotide sequence of
(SEQ ID NO: 4)
-(N)ₐUGAUGGA(N)_{b}CAAGGUUAAC(N)ₐ,
(SEQ ID NO: 5)
-(N)ₐCAAGGUUAAC(N)_{b}UGAUGGA(N)ₐ,
or
the two following sequences
(SEQ ID NO: 6)
-(N)ₐUGAUGGA(N)_{b},
(SEQ ID NO: 7)
(N)ₐCAAGGUUAAC(N)_{b},
wherein a, b and c are integer
a is higher than or equal to 4, preferably varies from 4 to 100,
b is higher than or equal to 1, preferably varies from 3 to 50,
c is higher than or equal to 1, preferably varies from 1 to 200,
or any variant of said nucleic acid molecule by substitution of at least one nucleic acid of one at least of said sequences SEQ ID NO: 4, SEQ ID NO: 5, SEQ ID NO: 6 and SEQ ID NO: 7, provided that said variant retains the ability to interact with said fluorophore and is able to induce fluorescence in aqueous solution.

9. The molecular complex according to claim 1, wherein the nucleic acid molecule comprises, or consists essentially of, or consists of one of the nucleotide sequences as set forth in SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27 and SEQ ID NO: 28.

10. The molecular complex according to claim 1, wherein said fluorophore is the fluorophore having one of the following formula 6 or 7, 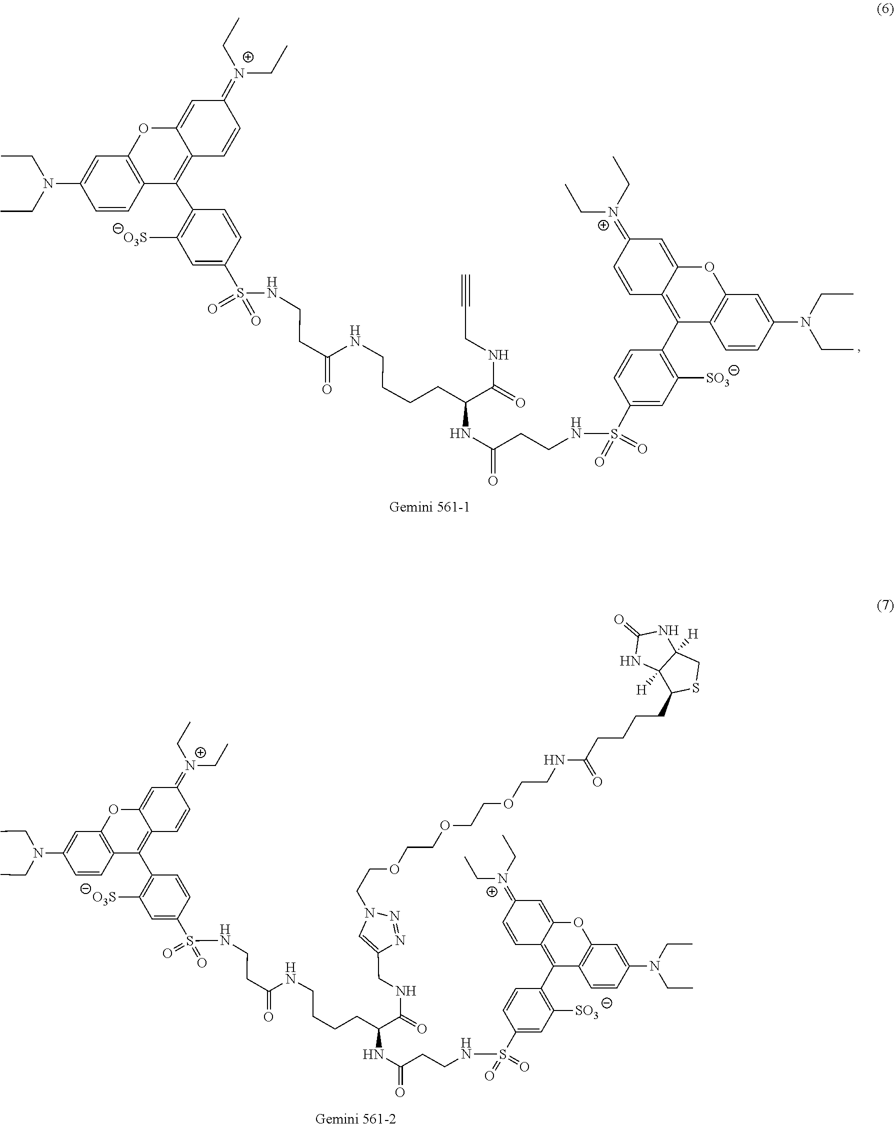
and
said nucleic acid comprises the sequence as set forth in SEQ ID NO: 8.

11. A nucleic acid molecule comprising a first and a second region, said first and second regions being such that:
the first region comprises the nucleotide sequence of SEQ ID NO: 1; and
the second region comprises the nucleotide sequence of SEQ ID NO: 2,
provided that said nucleic acid molecule is not the nucleic acid molecule as set forth in SEQ ID NO: 3.

12. The nucleic acid molecule according to claim 11, wherein the nucleic acid molecule comprises, or consists essentially of, or consists of one of the nucleotide sequences as set forth in SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, and SEQ ID NO: 29.

13. A host cell containing the nucleic acid molecule as defined in claim 11.

14. (canceled)

15. A method for imaging in vitro or ex vivo small molecules, RNA and proteins in cells, comprising the administration to a living cell in vivo and to an ex vivo cell culture,
a nucleic acid able to specifically interact, in a sequence specific manner, with a fluorophore operably linked to a biomolecule, along with the fluorophore molecule according to claim 1.


SEQUENCE LISTING (36 sequences)

SEQ ID NO: 1 - RNA, 7 nt, Artificial sequence
  fluorophore binding aptamers
ugaugga

SEQ ID NO: 2 - RNA, 10 nt, Artificial sequence
  fluorophore binding aptamers
caagguuaac

SEQ ID NO: 3 - RNA, 69 nt, Artificial sequence
  fluorophore binding aptamers
gggagacagcuagaguacggaaccucgcuucggcgaugauggacagguuccgacacgagcacaguguac

SEQ ID NO: 4 - RNA, 267 nt, Artificial sequence
  fluorophore binding aptamers
  misc_feature (1)..(96): n is a, c, g, u  or is absent
  misc_feature (97)..(100): n is a, c, g, or u
  misc_feature (108)..(154): n is a, c, g, u  or is absent
  misc_feature (155)..(157): n is a, c, g, or u
  misc_feature (168)..(171): n is a, c, g, or u
  misc_feature (172)..(267): n is a, c, g, u or is absent
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnugauggannnnnnnnnnnnnnnnnnnnnnnnnnnnnnnnnnnnnnnnnnnnnnnnnncaagguuaacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 5 - RNA, 417 nt, Artificial sequence
  fluorophore binding aptamers
  misc_feature (1)..(96): n is a, c, g, u  or is absent
  misc_feature (97)..(100): n is a, c, g or u
  misc_feature (111)..(111): n is a, c, g, or u
  misc_feature (112)..(310): n is a, c, g, u  or is absent
  misc_feature (318)..(321): n is a, c, g, or u
  misc_feature (322)..(417): n is a, c, g, u  or is absent
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaagguuaacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnugauggannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 6 - RNA, 157 nt, Artificial sequence
  fluorophore binding aptamers
  misc_feature (1)..(96): n is a, c, g, u  or is absent
  misc_feature (97)..(100): n is a, c, g or u
  misc_feature (108)..(108): n is a, c, g, or u
  misc_feature (109)..(157): n is a, c, g, u  or is absent
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnugauggannnnnnnnnnnnnnnnnnnnnnnnnnnnnnnnnnnnnnnnnnnnnnnnnn

SEQ ID NO: 7 - RNA, 160 nt, Artificial sequence
  fluorophore binding aptamers
  misc_feature (1)..(96): n is a, c, g, or u or is absent
  misc_feature (97)..(100): n is a, c, g, or u
  misc_feature (111)..(111): n is a, c, g, or u
  misc_feature (112)..(160): n is a, c, g, u or is absent
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaagguuaacnnnnnnnnnnnnnnnnnnnnnnnnnnnnnnnnnnnnnnnnnnnnnnnnnn

SEQ ID NO: 8 - RNA, 151 nt, Artificial sequence
  fluorophore binding aptamers
gggagacagcuagaguacaggaaccccgcuucggcggugauggagaggcgcaagguuaaccgccucagguuccggugacggggccucgcuucggcgaugauggagaggcgcaagguuaaccgccucagguucugacacgagcacaguguac

SEQ ID NO: 9 - RNA, 168 nt, Artificial sequence
  fluorophore binding aptamers
  misc_feature (55)..(72): N represents A, U, G or C
  misc_feature (73)..(114): N represents A, U, G or C or is absent
agaaccccgcuucggcggugauggagaggcgcaagguuaaccgccucagguuccnnnnnnnnnnnnnnnnnnnnnnnnnnnnnnnnnnnnnnnnnnnnnnnnnnnnnnnnnnnnggggccucgcuucggcgaugauggagaggcgcaagguuaaccgccucagguucu

SEQ ID NO: 10 - RNA, 167 nt, Artificial sequence
  fluorophore binding aptamers
  misc_feature (54)..(71): N represents A, U, G or C
  misc_feature (72)..(113): N represents A, U, G , C or is absent
ggaaccucgcuucggcgaugauggagaggcgcaagguuaaccgccccgguuccnnnnnnnnnnnnnnnnnnnnnnnnnnnnnnnnnnnnnnnnnnnnnnnnnnnnnnnnnnnnggaaccccgcuucugcggugauggagaggcgcaauguuaaccgccucagguucc

SEQ ID NO: 11 - RNA, 168 nt, Artificial sequence
  fluorophore binding aptamers
  misc_feature (55)..(72): N represents A, U, G or C
  misc_feature (73)..(114): N represents A, U, G, C or is absent
agaaccccgcuucggcggugauggagaggcgcaagguuaaccgccucagguuccnnnnnnnnnnnnnnnnnnnnnnnnnnnnnnnnnnnnnnnnnnnnnnnnnnnnnnnnnnnnggggccucgcuucggcgaugauggagaggcgcaagguuaaccgccucagguucc

SEQ ID NO: 12 - RNA, 168 nt, Artificial sequence
  fluorophore binding aptamers
  misc_feature (55)..(72): N represents A, U, G or C
  misc_feature (73)..(114): N represents A, U, G, C or is absent
ggaaccucgcuucggcgaugauggagaggcgcaagguuaaccgccucagguuccnnnnnnnnnnnnnnnnnnnnnnnnnnnnnnnnnnnnnnnnnnnnnnnnnnnnnnnnnnnnggaaccucgcuucggcgaugauggagaggcgcaagguuaaccgccucagguucc

SEQ ID NO: 13 - RNA, 168 nt, Artificial sequence
  fluorophore binding aptamers
  misc_feature (55)..(72): N represents A, U, G or C
  misc_feature (73)..(114): N represents A, U, G, C or is absent
ggaacuucgcuucggcgaugauggagaggcgcaagguuaaccgccucagguuccnnnnnnnnnnnnnnnnnnnnnnnnnnnnnnnnnnnnnnnnnnnnnnnnnnnnnnnnnnnnggaacuucgcuucggcgaugauggagaggcgcaagguuaaccgccucagguucc

SEQ ID NO: 14 - RNA, 168 nt, Artificial sequence
  fluorophore binding aptamers
  misc_feature (55)..(72): N represents A, U, G or C
  misc_feature (73)..(114): N represents A, U, G, C or is absent
gggaccccgcuucggcggugauggagaggcgcaagguuaaccgccucagguuccnnnnnnnnnnnnnnnnnnnnnnnnnnnnnnnnnnnnnnnnnnnnnnnnnnnnnnnnnnnnggaaccucgcuucggcgaugauggaggggcgcaagguuaaccgccucagguuuc

SEQ ID NO: 15 - RNA, 167 nt, Artificial sequence
  fluorophore binding aptamers
  misc_feature (54)..(71): N represents A, U, G or C
  misc_feature (71)..(113): N represents A, U, G, C or is absent
ggagccccgcuucggcggugauggagaggcgcaaggcuaaccgccucgguuccnnnnnnnnnnnnnnnnnnnnnnnnnnnnnnnnnnnnnnnnnnnnnnnnnnnnnnnnnnnnggagccucgcuucggcgaugauggagaggcgcaagguuaaccgccucagguucc

SEQ ID NO: 16 - RNA, 168 nt, Artificial sequence
  fluorophore binding aptamers
  misc_feature (55)..(72): N represents A, U, G or C
  misc_feature (73)..(114): N represents A, U, G, C or is absent
ggaaccucgcuucggcgaugauggagaggcgcaagguuaaccgccucagguuccnnnnnnnnnnnnnnnnnnnnnnnnnnnnnnnnnnnnnnnnnnnnnnnnnnnnnnnnnnnnggagccucgcuucggcgaugauggaggggcgcaagguuaaccgccucagguuca

SEQ ID NO: 17 - RNA, 168 nt, Artificial sequence
  fluorophore binding aptamers
  misc_feature (55)..(72): N represents A, U, G or C
  misc_feature (73)..(114): N represents A, U, G, C or is absent
ggaaccucacuucggugaugauggagaggcgcaagguuaaccgccucagguuccnnnnnnnnnnnnnnnnnnnnnnnnnnnnnnnnnnnnnnnnnnnnnnnnnnnnnnnnnnnnggagccucgcuucggcgaugauggagaggcgcaagguuaaccgccucagguucc

SEQ ID NO: 18 - RNA, 168 nt, Artificial sequence
  fluorophore binding aptamers
  misc_feature (55)..(72): N represents A, U, G or C
  misc_feature (73)..(114): N represents A, U, G, C or is absent
gggaccucguuucggcgaugauggagaggcgcaagguuaaccgccucagguuccnnnnnnnnnnnnnnnnnnnnnnnnnnnnnnnnnnnnnnnnnnnnnnnnnnnnnnnnnnnnggagccucgcuucggcgaugauggagaggcgcaagguuaaccgccucagguucc

SEQ ID NO: 19 - RNA, 168 nt, Artificial sequence
  fluorophore binding aptamers
  misc_feature (55)..(72): N represents A, U, G or C
  misc_feature (73)..(114): N represents A, U, G, C or is absent
ggagccucgcuuaggcgaugauggagaggcgcaagguuaaccgccucagguuccnnnnnnnnnnnnnnnnnnnnnnnnnnnnnnnnnnnnnnnnnnnnnnnnnnnnnnnnnnnnggaaccucgcuucggcgaugauggagaggcgcaagguuaaccgccucagguucc

SEQ ID NO: 20 - RNA, 168 nt, Artificial sequence
  fluorophore binding aptamers
  misc_feature (55)..(72): N represents A, U, G or C
  misc_feature (73)..(114): N represents A, U, G, C or is absent
ggaaccccgcuucgguggugauggagaggcgcaagguuaaccgcgucagguuccnnnnnnnnnnnnnnnnnnnnnnnnnnnnnnnnnnnnnnnnnnnnnnnnnnnnnnnnnnnnggaaccucgcuucggcgaugauggagaggcgcaagguuaaccgccucagguucc

SEQ ID NO: 21 - RNA, 168 nt, Artificial sequence
  fluorophore binding aptamers
  misc_feature (55)..(72): N represents A, U, G or C
  misc_feature (73)..(114): N represents A, U, G, C or is absent
ggaaccucgcuucggcgaugauggagaggcgcaagguuaaccgccucagguuccnnnnnnnnnnnnnnnnnnnnnnnnnnnnnnnnnnnnnnnnnnnnnnnnnnnnnnnnnnnnggaaucucgcuucggcgaugauggagaggcgcaagguuaaccgccucagguucc

SEQ ID NO: 22 - RNA, 167 nt, Artificial sequence
  fluorophore binding aptamers
  misc_feature (54)..(71): N represents A, U, G or C
  misc_feature (72)..(113): N represents A, U, G, C or is absent
ggaaccucgcuucggcgaugauggagaggcgcaagguuaaccgccucgguuccnnnnnnnnnnnnnnnnnnnnnnnnnnnnnnnnnnnnnnnnnnnnnnnnnnnnnnnnnnnnggaaucucgcuucggcgaugauggagaggcgcaagguuaaccgccucagguucc

SEQ ID NO: 23 - RNA, 167 nt, Artificial sequence
  fluorophore binding aptamers
  misc_feature (55)..(72): N represents A, U, G or C
  misc_feature (73)..(114): N represents A, U, G, C or is absent
ggaacuucgcuucggcgaugauggagaggcgcaagguuaaccgccucagguuccnnnnnnnnnnnnnnnnnnnnnnnnnnnnnnnnnnnnnnnnnnnnnnnnnnnnnnnnnnnnggaacuucgcuucggcgaugauggagaggcgcaagguuaaccgccucgguucc

SEQ ID NO: 24 - RNA, 54 nt, Artificial sequence
  fluorophore binding aptamers
gggaccucgcuucggcgaugauggagaggcacaagguuaacugccucagguucc

SEQ ID NO: 25 - RNA, 54 nt, Artificial sequence
  fluorophore binding aptamers
ggaaccucgcuucggcgaugauggagaggcacaagguuaacugccucagguucc

SEQ ID NO: 26 - RNA, 167 nt, Artificial sequence
  fluorophore binding aptamers
  misc_feature (54)..(71): N represents A, U, G or C
  misc_feature (72)..(113): N represents A, U, G, C or is absent
ggaaccucgcuucggcgaugauggagaggcgcaagguuaaccgccucaguuccnnnnnnnnnnnnnnnnnnnnnnnnnnnnnnnnnnnnnnnnnnnnnnnnnnnnnnnnnnnnggaacuucgcuucggcgaugauggagaggcgcaagguuaaccgccucagguucc

SEQ ID NO: 27 - RNA, 53 nt, Artificial sequence
  fluorophore binding aptamers
ggaaccucgcuucggcgaugauggagaggcgcaauguuaaccgccucgguucc

SEQ ID NO: 28 - RNA, 167 nt, Artificial sequence
  fluorophore binding aptamers
  misc_feature (54)..(71): N represents A, U, G or C
  misc_feature (72)..(113): N represents A, U, G, C or is absent
ggaaccucgcuucggcgaugauggaggggcgcaagguuaaccgccucgguuccnnnnnnnnnnnnnnnnnnnnnnnnnnnnnnnnnnnnnnnnnnnnnnnnnnnnnnnnnnnnggaaccucgcuucggcgaugauggagaggcgcaagguuaaccgccucagguucc

SEQ ID NO: 29 - RNA, 283 nt, Artificial sequence
  fluorophore binding aptamers
gugcucgcuucggcagcacauauacuagucgacuugccauguguaugugggccugcagggggagacagcuagaguacagaaccccgcuucggcggugauggagaggcgcaagguuaaccgccucagguuccggugacggggccucgcuucggcgaugauggagaggcgcaagguuaaccgccucagguucugacacgagcacaguguacccugcaggcccacauacucugaugauccuucgggaucauucauggcaaucuagagcggacuucgguccgcuuuu

SEQ ID NO: 30 - DNA, 18 bp, Artificial sequence
  sense oligonucleotide
gggagacagctagagtac

SEQ ID NO: 31 - DNA, 18 bp, Artificial sequence
  antisense oligonucleotide
gacacgagcacagtgtac

SEQ ID NO: 32 - DNA, 38 bp, Artificial sequence
  sense oligonucleotide
ctttaatacgactcactatagggagacagctagagtac

SEQ ID NO: 33 - DNA, 18 bp, Artificial sequence
  antisense oligonucleotide
gacacgagcacagtgtac

SEQ ID NO: 34 - DNA, 86 bp, Artificial Sequence
  SIR-A coding sequence
gggagacagctagagtacggccaccgggtttgaaaacctggctgcttcggcagttgtatcctttggccgacacgagcacagtgtac

SEQ ID NO: 35 - DNA, 38 bp, Artificial Sequence; identical to SEQ ID NO: 32
  sens oligonucleotode for SEQ ID NO 34

SEQ ID NO: 36 - DNA, 18 bp, Artificial Sequence
  oligo reverse for SEQ ID NO 34
gtacactgtgctcgtgtc